(19) United States
(12) Patent Application Publication
(10) Pub. No.: US 2024/0300968 A1
(43) Pub. Date: Sep. 12, 2024

(54) 6-SUBSTITUTED NAPHTHALENE-1,3-DISULFONIC ACID DERIVATIVES AS MODULATORS OF THE EXTRACELLULAR NICOTINAMIDE PHOSPHORIBOSYL TRANSFERASE (ENAMPT) FOR THE TREATMENT OF E.G. DIABETES

(71) Applicant: The University of Manchester, Manchester (GB)

(72) Inventors: Samuel Butterworth, Manchester (GB); Miguel Garzon Sanz, Madrid (ES)

(21) Appl. No.: 18/291,809

(22) PCT Filed: Jul. 26, 2022
(86) PCT No.: PCT/GB2022/051955

§ 371 Date: Jan. 24, 2024

(30) Foreign Application Priority Data
Jul. 26, 2021 (GB) 2110728.9

(51) Int. Cl. C07D 491/08; A61K 31/17; A61K 31/4035; A61K 31/4192; A61K 31/4453; A61K 31/454; A61K 31/5386; C07C 317/42; C07D 211/34; C07D 211/96; C07D 249/06; C07D 401/12; C07D 405/12

(52) U.S. Cl. CPC C07D 491/08; A61K 31/17; A61K 31/4035; A61K 31/4192; A61K 31/4453; A61K 31/454; A61K 31/5386; C07C 317/42; C07D 211/34; C07D 211/96; C07D 249/06; C07D 401/12; C07D 405/12

(57) ABSTRACT

This invention relates to therapeutic 6-substituted naphthalene-1,3-disulfonic acid derivatives of formula (I) (Formula (I)). More specifically, the invention relates to compounds of formula (I) useful as modulators of extracellular nicotinamide phosphoribosyl transferase (eNAMPT) that stabilize the protein in its dimeric form. In addition the invention contemplates pharmaceutical compositions comprising the compounds, processes to prepare the compounds and the compounds for use in methods of medical treatment of e.g. (i) diabetes; (ii) cardiovascular disease; (iii) inflammatory bowel condition; (iv) cancer; (v) liver disease; (vi) inflammatory skin conditions; (vii) lung conditions; (viii) arthritis; (ix) kidney disease (e.g. chronic kidney disease); or (x) sepsis. An exemplary compound is e.g. 6-(2-fluoro-5-((3-(4-(piperidin-1-ylsulfonyl) phenyl) ureido)methyl)phenyl)-4-(trioxidaneylthio) naphthalene-2-sulfonic acid (example 1).

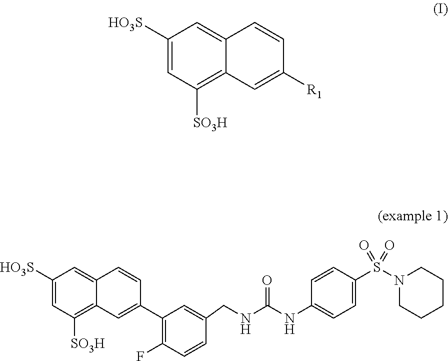

### FIELD OF INVENTION

This invention relates to therapeutic compounds. More specifically, the invention relates to compounds that suppress extracellular nicotinamide phosphoribosyl transferase (eNAMPT) activity. In addition, the invention contemplates pharmaceutical compositions comprising the compounds, processes to prepare the compounds and uses of the compounds.

### BACKGROUND

Extracellular nicotinamide phosphoribosyl transferase has been shown to act as a mediator of inflammation in a number of indications including diabetes, inflammatory bowel disease (IBD), non-alcoholic fatty liver disease (NAFLD), pulmonary arterial hypertension (PAH), acute lung injury (ALI), radiation-induced lung injury (RILI), cardiovascular disease, rheumatoid arthritis and cancer, among others. Activating polymorphisms in the NAMPT gene loci have been linked to increased IL-6 levels, and worse outcomes or more severe disease in acute lung injury and pulmonary arterial hypertension and cardiovascular disease,

The nicotinamide phosphoribosyl transferase homodimer has enzymatic activity, both in synthesis of the nicotinamide adenine dinucleotide (NAD⁺) precursor nicotinamide mononucleotide (NAD) and hydrolysis/exchange of adenosine triphosphate (ATP) to form adenosine diphosphate (ADP) and adenosine tetraphosphate. However studies using enzymatically inactive monomeric mutants have demonstrated that the pro-inflammatory effects of eNAMPT are not driven by this activity. This supports the hypothesis that eNAMPT acts via binding to one or more cell receptors. Further evidence for this is provided by the fact that eNAMPT can directly bind to both TLR4 and CCR5, with TLR4 function being essential to the proinflammatory effects of exogenously administered eNAMPT in mice.

Proinflammatory monomeric eNAMPT levels are raised in patients with type 2 diabetes and the monomeric protein is also released by tumour cells in vitro, however monomer/dimer ratio has not been routinely examined across all disease areas where eNAMPT is implicated in disease pathogenesis.

Monoclonal antibodies that block the proinflammatory effects of eNAMPT have been shown to have beneficial effects in cellular and animal models of diabetes, inflammatory bowel disease (IBD), pulmonary arterial hypertension (PAH), acute lung injury (ALI), and radiation-induced lung injury (RILI).

Small-molecule NAMPT inhibitors have been studied in a range of disease settings, especially cancer, due to their ability to reduce cellular NAD⁺ levels, resulting in changes in cellular phenotype and induction of cell death. However, due to the relative non-specificity of these molecules across different cell types they tend to show a negligible therapeutic window; to date compounds have only been advanced to clinical trials in oncology indications and none have shown a useable therapeutic window.

### AIMS OF THE INVENTION

It is an aim of certain embodiments of this invention to provide compounds that suppress the proinflammatory effects of monomeric extracellular nicotinamide phosphoribosyl transferase (eNAMPT), optionally by stabilizing the protein in a dimeric form and suppressing the formation of monomeric eNAMPT and/or inducing a conformational change in the protein.

It is a further aim of certain embodiments of this invention to provide compounds that selectively suppress the proinflammatory effects of extracellular NAMPT, without demonstrating any significant inhibition of intracellular NAMPT activity.

The present invention was devised with the foregoing in mind.

### SUMMARY OF THE INVENTION

In one aspect, the present invention provides a compound as defined herein, or a pharmaceutically acceptable salt or solvate thereof.

In another aspect, the present invention provides a compound as defined herein, or a pharmaceutically acceptable salt or solvate thereof, for use as a medicament.

In another aspect, the present invention provides a compound as defined herein, or a pharmaceutically acceptable salt or solvate thereof, for use in the treatment of a disease or condition in which the suppression of eNAMPT activity is beneficial. Suitably, a compound as defined herein, or a pharmaceutically acceptable salt or solvate thereof, is for use in the treatment of a disease or condition in which the suppression of monomeric eNAMPT activity is beneficial.

In another aspect, the present invention provides a compound as defined herein, or a pharmaceutically acceptable salt or solvate thereof, for use in the treatment of:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH));
(vi) inflammatory skin conditions (e.g. psoriasis);
(vii) lung conditions (Acute Lung Injury (ALI), Ventilator Induced Lung Injury (VILI), Radiation-Induced Lung Injury (RILI), pulmonary fibrosis (e.g. idiopathic pulmonary fibrosis));
(viii) arthritis (e.g. osteoarthritis or rheumatoid arthritis);
(ix) kidney disease (e.g. chronic kidney disease); or
(x) sepsis.

In another aspect, the present invention provides a compound as defined herein, or a pharmaceutically acceptable salt or solvate thereof, for use in the treatment of:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH), hepatic steatosis, fibrosis or cirrhosis);
(vi) lung conditions (Acute Lung Injury (ALI), Ventilator Induced Lung Injury (VILI), Radiation-Induced Lung Injury (RILI), pulmonary fibrosis (e.g. idiopathic pulmonary fibrosis));
(vii) kidney disease (e.g. chronic kidney disease).

In another aspect, the present invention provides a compound as defined herein, or a pharmaceutically acceptable salt or solvate thereof, for use in the treatment of:
(i) diabetes;
(ii) pulmonary arterial hypertension, acute coronary syndrome (ACS), congestive heart failure (CHF) or left ventricular hypertrophy;
(iii) inflammatory bowel disease (IBD), Crohn's Disease or ulcerative colitis;
(iv) breast cancer, prostate cancer or chronic lymphocytic leukaemia (CLL)
(v) non-alcoholic fatty liver disease (NAFLD), in particular Non-Alcoholic SteatoHepatitis (NASH), hepatic steatosis, fibrosis or cirrhosis);
(vi) psoriasis;
(vii) Acute Lung Injury (ALI), Ventilator Induced Lung Injury (VILI), Radiation-Induced Lung Injury (RILI), pulmonary fibrosis (e.g. idiopathic pulmonary fibrosis);
(viii) osteoarthritis or rheumatoid arthritis;
(ix) chronic kidney disease; or
(x) sepsis.

In another aspect, the present invention provides a compound as defined herein, or a pharmaceutically acceptable salt or solvate thereof, for use in the treatment of:
(i) diabetes;
(ii) pulmonary arterial hypertension;
(iii) inflammatory bowel disease (IBD), Crohn's Disease or ulcerative colitis;
(iv) non-alcoholic fatty liver disease (NAFLD), in particular Non-Alcoholic SteatoHepatitis (NASH);
(v) Acute Lung Injury (ALI), Ventilator Induced Lung Injury (VILI), Radiation-Induced Lung Injury (RILI); or
(vi) chronic kidney disease.

In another aspect, the present invention provides a compound as defined herein, or a pharmaceutically acceptable salt or solvate thereof, for use in the treatment of inflammatory bowel disease (IBD), Crohn's Disease or ulcerative colitis.

In another aspect, the present invention provides a compound as defined herein, or a pharmaceutically acceptable salt or solvate thereof, for use in the treatment of diabetes.

In another aspect, the present invention provides a compound as defined herein, or a pharmaceutically acceptable salt or solvate thereof, for use in the treatment of pulmonary arterial hypertension.

In another aspect, the present invention provides a compound as defined herein, or a pharmaceutically acceptable salt or solvate thereof, for use in the treatment of non-alcoholic fatty liver disease (NAFLD), in particular Non-Alcoholic SteatoHepatitis (NASH).

In another aspect, the present invention provides a compound as defined herein, or a pharmaceutically acceptable salt or solvate thereof, for use in the treatment of Acute Lung Injury (ALI), Ventilator Induced Lung Injury (VILI), or Radiation-Induced Lung Injury (RILI).

In another aspect, the present invention provides a compound as defined herein, or a pharmaceutically acceptable salt or solvate thereof, for use in the treatment of chronic kidney disease.

Suitably, the compound as defined herein, or a pharmaceutically acceptable salt or solvate thereof, is for use in the treatment of pulmonary arterial hypertension, IBD, crohn's disease, ulcerative colitis, diabetes (in particular in subjects with cardiovascular disease comorbidities), chronic kidney disease, ventilator induced lung injury (VILI) (e.g. in subjects who have been treated for COVID-19), acute lung injury (ALI), acute respiratory distress syndrome (ARDS), idiopathic pulmonary fibrosis, non-alcoholic fatty liver disease (including but not limited to hepatic steatosis, through inflammatory non-alcoholic steatohepatitis (NASH)), to fibrosis or cirrhosis) or radiation induced lung injury (RILI).

In another aspect, the present invention provides a compound as defined herein, or a pharmaceutically acceptable salt or solvate thereof, or a pharmaceutical composition as defined herein, for use in the suppression of eNAMPT activity. Suitably, the present invention provides a compound as defined herein, or a pharmaceutically acceptable salt or solvate thereof, or a pharmaceutical composition as defined herein, for use in the suppression of monomeric eNAMPT activity.

In another aspect, the present invention provides a method of suppressing eNAMPT activity in vitro or in vivo, said method comprising contacting a a sample comprising eNAMPT with an effective amount of a compound as defined herein, or a pharmaceutically acceptable salt or solvate thereof. Suitably, the method is a method of suppressing monomeric eNAMPT activity.

In a further aspect, the present invention provides a method of treating:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH));
(vi) inflammatory skin conditions (e.g. psoriasis);
(vii) lung conditions (Acute Lung Injury (ALI), Ventilator Induced Lung Injury (VILI), Radiation-Induced Lung Injury (RILI), pulmonary fibrosis (e.g. idiopathic pulmonary fibrosis));
(viii) arthritis (e.g. osteoarthritis or rheumatoid arthritis);
(ix) kidney disease (e.g. chronic kidney disease); or
(x) sepsis;
   in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH), hepatic steatosis, fibrosis or cirrhosis);
(vi) lung conditions (Acute Lung Injury (ALI), Ventilator Induced Lung Injury (VILI), Radiation-Induced Lung Injury (RILI), pulmonary fibrosis (e.g. idiopathic pulmonary fibrosis)); or
(vii) kidney disease (e.g. chronic kidney disease);
   in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating:
(i) diabetes;
(ii) pulmonary arterial hypertension, acute coronary syndrome (ACS), congestive heart failure (CHF) or left ventricular hypertrophy;
(iii) inflammatory bowel disease (IBD), Crohn's Disease or ulcerative colitis;
(iv) breast cancer, prostate cancer or chronic lymphocytic leukaemia (CLL)
(v) non-alcoholic fatty liver disease (NAFLD), in particular Non-Alcoholic SteatoHepatitis (NASH), hepatic steatosis, fibrosis or cirrhosis);
(vi) psoriasis;
(vii) Acute Lung Injury (ALI), Ventilator Induced Lung Injury (VILI), Radiation-Induced Lung Injury (RILI), pulmonary fibrosis (e.g. idiopathic pulmonary fibrosis);
(viii) osteoarthritis or rheumatoid arthritis;
(ix) chronic kidney disease; or
(x) sepsis;
   in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating:
(i) diabetes;
(ii) pulmonary arterial hypertension;
(iii) inflammatory bowel disease (IBD), Crohn's Disease or ulcerative colitis;
(iv) non-alcoholic fatty liver disease (NAFLD), in particular Non-Alcoholic SteatoHepatitis (NASH);
(v) Acute Lung Injury (ALI), Ventilator Induced Lung Injury (VILI), Radiation-Induced Lung Injury (RILI); or
(vi) chronic kidney disease;
   in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating inflammatory bowel disease (IBD), Crohn's Disease or ulcerative colitis in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating diabetes in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating pulmonary arterial hypertension in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating non-alcoholic fatty liver disease (NAFLD), in particular Non-Alcoholic SteatoHepatitis (NASH) in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating chronic kidney disease in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, the present invention provides the use of a compound as defined herein, or a pharmaceutically acceptable salt thereof, in the manufacture of a medicament for use in the treatment of:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH));
(vi) inflammatory skin conditions (e.g. psoriasis);
(vii) lung conditions (Acute Lung Injury (ALI), Ventilator Induced Lung Injury (VILI), Radiation-Induced Lung Injury (RILI), pulmonary fibrosis (e.g. idiopathic pulmonary fibrosis));
(viii) arthritis (e.g. osteoarthritis or rheumatoid arthritis);
(ix) kidney disease (e.g. chronic kidney disease); or
(x) sepsis.

In another aspect, the present invention provides the use of a compound as defined herein, or a pharmaceutically acceptable salt thereof, in the manufacture of a medicament for use in the treatment of:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH), hepatic steatosis, fibrosis or cirrhosis);
(vi) lung conditions (Acute Lung Injury (ALI), Ventilator Induced Lung Injury (VILI), Radiation-Induced Lung Injury (RILI), pulmonary fibrosis (e.g. idiopathic pulmonary fibrosis));
(vii) kidney disease (e.g. chronic kidney disease).

In another aspect, the present invention provides the use of a compound as defined herein, or a pharmaceutically acceptable salt thereof, in the manufacture of a medicament for use in the treatment of:
(i) diabetes;
(ii) pulmonary arterial hypertension, acute coronary syndrome (ACS), congestive heart failure (CHF) or left ventricular hypertrophy;
(iii) inflammatory bowel disease (IBD), Crohn's Disease or ulcerative colitis;
(iv) breast cancer, prostate cancer or chronic lymphocytic leukaemia (CLL)
(v) non-alcoholic fatty liver disease (NAFLD), in particular Non-Alcoholic SteatoHepatitis (NASH), hepatic steatosis, fibrosis or cirrhosis);
(vi) psoriasis;
(vii) Acute Lung Injury (ALI), Ventilator Induced Lung Injury (VILI), Radiation-Induced Lung Injury (RILI), pulmonary fibrosis (e.g. idiopathic pulmonary fibrosis);
(viii) osteoarthritis or rheumatoid arthritis;
(ix) chronic kidney disease; or
(x) sepsis.

In another aspect, the present invention provides the use of a compound as defined herein, or a pharmaceutically acceptable salt thereof, in the manufacture of a medicament for use in the treatment of:
(i) diabetes;
(ii) pulmonary arterial hypertension;
(iii) inflammatory bowel disease (IBD), Crohn's Disease or ulcerative colitis;
(iv) non-alcoholic fatty liver disease (NAFLD), in particular Non-Alcoholic SteatoHepatitis (NASH);
(v) Acute Lung Injury (ALI), Ventilator Induced Lung Injury (VILI), Radiation-Induced Lung Injury (RILI); or
(vi) chronic kidney disease.

In another aspect, the present invention provides the use of a compound as defined herein, or a pharmaceutically acceptable salt thereof, in the manufacture of a medicament for use in the treatment of inflammatory bowel disease (IBD), Crohn's Disease or ulcerative colitis.

In another aspect, the present invention provides the use of a compound as defined herein, or a pharmaceutically acceptable salt thereof, in the manufacture of a medicament for use in the treatment of diabetes.

In another aspect, the present invention provides the use of a compound as defined herein, or a pharmaceutically acceptable salt thereof, in the manufacture of a medicament for use in the treatment of pulmonary arterial hypertension.

In another aspect, the present invention provides the use of a compound as defined herein, or a pharmaceutically acceptable salt thereof, in the manufacture of a medicament for use in the treatment of non-alcoholic fatty liver disease (NAFLD), in particular Non-Alcoholic SteatoHepatitis (NASH).

In another aspect, the present invention provides the use of a compound as defined herein, or a pharmaceutically acceptable salt thereof, in the manufacture of a medicament for use in the treatment of Acute Lung Injury (ALI), Ventilator Induced Lung Injury (VILI), or Radiation-Induced Lung Injury (RILI).

In another aspect, the present invention provides the use of a compound as defined herein, or a pharmaceutically acceptable salt thereof, in the manufacture of a medicament for use in the treatment of chronic kidney disease.

In another aspect, the present invention provides the use of a compound as defined herein, or a pharmaceutically acceptable salt thereof, in the manufacture of a medicament for the suppression of eNAMPT activity.

In another aspect, the present invention provides a pharmaceutical composition as defined herein which comprises a compound as defined herein, or a pharmaceutically acceptable salt or solvate thereof, and one or more pharmaceutically acceptable excipients.

Preferred, suitable, and optional features of any one particular aspect of the present invention are also preferred, suitable, and optional features of any other aspect.

In one aspect, the present invention provides a combination comprising a compound as defined herein, or a pharmaceutically acceptable salt thereof, with one or more additional therapeutic agents.

The present invention further provides a method of synthesising a compound, or a pharmaceutically acceptable salt, as defined herein.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups and analogues thereof. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like.

“(2-6C)alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkenyl” means a hydrocarbon ring containing at least one double bond, for example, cyclobutenyl, cyclopentenyl, cyclohexenyl or cycloheptenyl, such as 3-cyclohexen-1-yl, or cyclooctenyl.

“(3-8C)cycloalkyl-(1-6C)alkylene” means a (3-8C)cycloalkyl group covalently attached to a (1-6C)alkylene group, both of which are defined herein.

The term “halo” or “halogeno” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). The term heterocyclyl includes both monovalent species and divalent species. Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocycles contain from about 7 to about 17 ring atoms, suitably from 7 to 12 ring atoms. Bicyclic heterocyclic(s) rings may be fused, spiro, or bridged ring systems.

Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

Suitably, a nitrogen atom in a heterocyclic ring system may be in the form of alkylammonium salt, for example a dimethylpiperidin-1-ium salt:

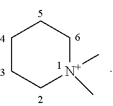

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

“Heterocyclyl(1-6C)alkyl” means a heterocyclyl group covalently attached to a (1-6C)alkylene group, both of which are defined herein.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-6C)alkyl” means a heteroaryl group covalently attached to a (1-6C)alkylene group, both of which are defined herein. Examples of heteroaralkyl groups include pyridin-3-ylmethyl, 3-(benzofuran-2-yl)propyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-6C)alkyl” means an aryl group covalently attached to a (1-6C)alkylene group, both of which are defined herein. Examples of aryl-(1-6C)alkyl groups include benzyl, phenylethyl, and the like.

This specification also makes use of several composite terms to describe groups comprising more than one functionality. Such terms will be understood by a person skilled in the art. For example heterocyclyl(m-nC)alkyl comprises (m-nC)alkyl substituted by heterocyclyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In one aspect, the present invention relates to a compound of formula I or a pharmaceutically acceptable salt or solvate thereof, as shown below:

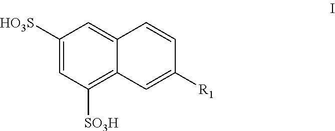

wherein R₁ is selected from:
(i) a group of the formula II:

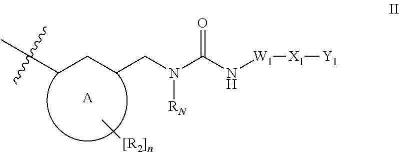

wherein:
Ring A is selected from phenyl or a 5- or 6-membered heteroaryl;
n is 0, 1 or 2;
each R₂ group, when present, is selected from: halo, nitro, cyano, R₂ₐ, —[CH₂]_{q}—NR₂ₐR_{2b}, —[CH₂]_{q}—OR₂ₐ, —[CH₂]_{q}—C(O)R₂ₐ, —[CH₂]_{q}—C(O)OR₂ₐ, —[CH₂]_{q}—OC(O)R₂ₐ, —[CH₂]_{q}—C(O)N(R_{2b})R₂ₐ, —[CH₂]_{q}—N(R_{2b})C(O)R₂ₐ, —[CH₂]_{q}—N(R_{2c})—C(O)—N(R_{2b})R₂ₐ, —[CH₂]_{q}—S(O)ₚR₂ₐ (where p is 0, 1 or 2), —[CH₂]_{q}—SO₂N(R₂)R₂ₐ, —[CH₂]_{q}—N(R_{2b})SO₂R₂ₐ;
   wherein q is 0, 1, 2 or 3;
   R₂ₐ is hydrogen or (1-4C)alkyl optionally substituted by halo, hydroxy, amino or cyano; and
   R_{2b} and R_{2c} are hydrogen or (1-2C)alkyl;
R_{N} is selected from hydrogen or methyl;
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, (1-2C)alkyl, (1-2C)haloalkyl, cyano or amino;
W₁ is:

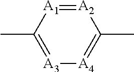

wherein A₁, A₂, A₃ or A₄ are selected from CH, N or C—F, with the provisos that:
   only one or two of A₁, A₂, A₃ or A₄ can be N; and
   only one or two of A₁, A₂, A₃ or A₄ can be C—F;
X₁ is a linker group of the formula:

—[CH₂]ₙ₁-L₁-[CH₂]ₙ₂—
wherein
n1 and n2 are selected from 0 or 1;
L₁ is selected from —O—, —S—, —SO—, —SO₂—, —N(Rₓ₁ₐ)—, —C(O)—, —C(O)O—, —OC(O)—, —C(O)N(Rₓ₁ₐ)—, —N(Rₓ₁ₐ)C(O)—, —N(R_{x1b})C(O)N(Rₓ₁ₐ)—, —N(Rₓ₁ₐ)C(O)O—, —OC(O)N(Rₓ₁ₐ)—, —S(O)₂N(Rₓ₁ₐ), —N(Rₓ₁ₐ)SO₂—, or —C(O)N(Rₓ₁ₐ)SO₂—, or —SO₂N(Rₓ₁ₐ)C(O)—; and wherein Rₓ₁ₐ and R_{x1b} are each independently selected from hydrogen or (1-2C)alkyl;
Y₁ is selected from a carbocyclic, aryl, heteroaryl or heterocyclic ring which is optionally substituted by halo, nitro, cyano, R_{y1a}, —[CH₂]ᵣ—NR_{y1a}R_{y1b}, —[CH₂]ᵣ—OR_{y1a}, —[CH₂]ᵣ—C(O)R_{y1a}, —[CH₂]ᵣ—C(O)OR_{y1a}, —[CH₂]ᵣ—OC(O)R_{y1a}, —[CH₂]ᵣ—C(O)N(R_{y1b})R_{y1a}, —[CH₂]ᵣ—N(R_{y1b})C(O)R_{y1a}, —[CH₂]ᵣ—N(R_{y1c})—C(O)—N(R_{y1b})R_{y1a}, —[CH₂]ᵣ—S(O)ₚR_{y1a} (where p is 0, 1 or 2), —[CH₂]ᵣ—SO₂N(R_{y1b})R_{y1a}, or —[CH₂]ᵣ—N(R_{y1b})SO₂R_{y1a};
   wherein r is 0, 1, 2 or 3;
   R_{y1} is hydrogen or (1-4C)alkyl optionally substituted by halo, hydroxy, amino or cyano; and
   R_{y1b} and R_{y1c} are hydrogen or (1-2C)alkyl;
(ii) a group of the formula III:

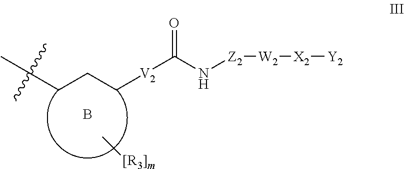

wherein:
Ring B is selected from phenyl or a 5- or 6-membered heteroaryl;
m is 0, 1 or 2;
each R₃ group, when present, is selected from: halo, nitro, cyano, R₃ₐ, —[CH₂]_{q1}—NR₃ₐR_{3b}, —[CH₂]_{q1}—OR₃ₐ, —[CH₂]_{q1}—C(O)R₃ₐ, —[CH₂]_{q1}—C(O)OR₃ₐ, —[CH₂]_{q1}—OC(O)R₃ₐ, —[CH₂]_{q1}—C(O)N(R_{3b})R₃ₐ, —[CH₂]_{q1}—N(R_{3b})C(O)R₃ₐ, —[CH₂]_{q1}—N(R_{3c})—C(O)—N(R_{3b})R₃ₐ, —[CH₂]_{q1}—S(O)ₚR₃ₐ (where p is 0, 1 or 2), —[CH₂]_{q1}—SO₂N(R_{3b})R₃ₐ, —[CH₂]_{q1}—N(R_{3b})SO₂R₃ₐ;
   wherein q1 is 0, 1, 2 or 3;
   R₃ₐ is hydrogen or (1-4C)alkyl optionally substituted by halo, hydroxy, amino or cyano; and
   R_{3b} and R_{3c} are hydrogen or (1-2C)alkyl;
V₂ is selected from —C(Rᵥ₂ₐR_{v2b})C(R_{2c}R_{v2d})— or —C(Rᵥ₂ₐ)═C(R_{v2c})—, wherein Rᵥ₂ₐ, R_{v2b}, R_{v2c} and R_{v2d} are each independently selected from hydrogen or methyl or Rᵥ₂ₐ and R_{v2c} are linked to form a cyclopropyl ring;
Z₂ is a 4C alkylene linker which is optionally substituted by one or more fluoro atoms;
W₂ is a carbocyclic, aryl, heteroaryl or heterocyclic ring which is optionally substituted by halo, nitro, cyano, R_{w2a}, —[CH₂]ₛ—NR_{w2a}R_{w2b}, —[CH₂]ₛ—OR_{w2a}, —[CH₂]ₛ—C(O)R_{w2a}, —[CH₂]ₛ—C(O)OR_{w2a}, —[CH₂]ₛ—OC(O)R_{w2a}, —[CH₂]ₛ—C(O)N(R_{w2b})R_{w2a}, —[CH₂]ₛ—N(R_{w2b})C(O)R_{w2a}, —[CH₂]ₛ—N(R_{w2c})—C(O)—N(R_{w2b})R_{w2a}, —[CH₂]ₛ—S(O)ₚR_{w2a} (where p is 0, 1 or 2), —[CH₂]ₛ—SO₂N(R_{w2b})R_{w2a}, or —[CH₂]ₛ—N(R_{w2b})SO₂R_{w2a}; wherein
   s is 0, 1 or 2;
   R_{w2a} is hydrogen or (1-4C)alkyl optionally substituted by halo, hydroxy, amino or cyano; and
   R_{w2b} and R_{w2c} are hydrogen or (1-2C)alkyl;
X₂ is a linker group of the formula:

—[CH₂]ₘ₁-L₂-[CH₂]ₘ₂—
wherein
m1 and m2 are selected from 0 or 1;
L₂ is selected from —O—, —S—, —SO—, —SO₂—, —N(Rₓ₂ₐ)—, —C(O)—, —C(O)O—, —OC(O)—, —C(O)N(Rₓ₂ₐ)—, —N(Rₓ₂ₐ)C(O)—, —N(R_{x2b})C(O)N(Rₓ₂ₐ)—, —N(Rₓ₂ₐ)C(O)O—, —OC(O)N(Rₓ₂ₐ)—, —S(O)₂N(Rₓ₂ₐ), N(Rₓ₂ₐ)SO₂, or C(O)N(Rₓ₂ₐ)SO₂, or —SO₂N(Rₓ₂ₐ)C(O)—; and wherein Rₓ₂ₐ and R_{x2b} are each independently selected from hydrogen or (1-2C)alkyl;
Y₂ is selected from a carbocyclic, aryl, heteroaryl or heterocyclic ring which is optionally substituted by halo, nitro, cyano, R_{y2a}, —[CH₂]ₜ—NR_{y2a}R_{y2b}, —[CH₂]ₜ—OR_{y2a}, —[CH₂]ₜ—C(O)R_{y2a}, —[CH₂]ₜ—C(O)OR_{y2a}, —[CH₂]ₜ—OC(O)R_{y2a}, —[CH₂]ₜ—C(O)N(R_{y2b})R_{y2a}, —[CH₂]ₜ—N(R_{y2b})C(O)R_{y2a}, —[CH₂]ₜ—N(R_{y2c})—C(O)—N(R_{y2b})R_{y2a}, —[CH₂]ₜ—S(O)ₚR_{y2a} (where p is 0, 1 or 2), —[CH₂]ₜ—SO₂N(R_{y2b})R_{y2a}, or —[CH₂]ₜ—N(R_{y2b})SO₂R_{y2a};
   wherein t is 0, 1, 2 or 3;
   R_{y2a} is hydrogen or (1-4C)alkyl optionally substituted by halo, hydroxy, amino or cyano; and
   R_{y2b} and R_{y2c} are hydrogen or (1-2C)alkyl;
(iii) a group of the formula IV:

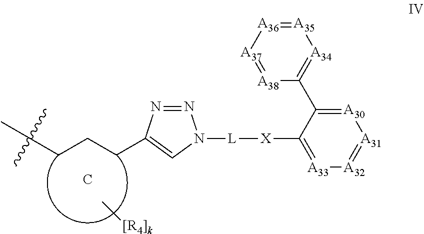

wherein:
Ring C is selected from phenyl, a 5- or 6-membered heteroaryl;
k is 0, 1 or 2;
each R₄ group, when present, is selected from: halo, nitro, cyano, R₄ₐ, —[CH₂]_{q2}—NR₄ₐR_{4b}, —[CH₂]_{q2}—OR₄ₐ, —[CH₂]_{q2}—C(O)R₄ₐ, —[CH₂]_{q2}—C(O)OR₄ₐ, —[CH₂]_{q2}—OC(O)R₄ₐ, —[CH₂]_{q2}—C(O)N(R_{4b})R₄ₐ, —[CH₂]_{q2}—N(R_{4b})C(O)R₄ₐ, —[CH₂]_{q2}—N(R_{4c})—C(O)—N(R_{4b})R₄ₐ, —[CH₂]_{q2}—S(O)ₚR₄ₐ (where p is 0, 1 or 2), —[CH₂]_{q2}—SO₂N(R_{4b})R₄ₐ, —[CH₂]_{q2}—N(R_{4b})SO₂R₄ₐ;
   wherein q2 is 0, 1, 2 or 3;
   R₄ₐ is hydrogen or (1-4C)alkyl optionally substituted by halo, hydroxy, amino or cyano; and
   R_{4b} and R_{4c} are hydrogen or (1-2C)alkyl;
L is a 6C alkylene linker which is optionally substituted by one or more fluoro atoms;
X is selected from —O—, —CRₓₐR_{xb}—, —S—, —SO—, —SO₂—, —N(Rₓₐ)—, —C(O)—; and wherein Rₓₐ and R_{xb} are each independently selected from hydrogen or methyl;
A₃₀ and A₃₁ are selected from CH, N or C—F;
A₃₂ and A₃₃ are selected from CH or N;
with the proviso that only one or two of A₃₀, A₃₁, A₃₂ and A₃₃ can be N;
A₃₄ and A₃₅ are selected from CH, N or C—R₃₀;
A₃₆, A₃₇ and A₃₈ are selected from CH or N;
with the proviso that only one or two of A₃₄, A₃₅, A₃₆, A₃₇ and A₃₈ can be N;
and wherein R₃₀ is selected from halo, nitro, cyano, R₃₀ₐ, —[CH₂]ₜ—NR₃₀ₐR_{30b}, —[CH₂]ₜ—OR₃₀ₐ, —[CH₂]ₜ—C(O)R₃₀ₐ, —[CH₂]ₜ—C(O)OR₃₀ₐ, —[CH₂]ₜ—OC(O)R₃₀ₐ, —[CH₂]ₜ—C(O)N(R_{30b})R₃₀ₐ, —[CH₂]ₜ—N(R_{30b})C(O)R₃₀ₐ, —[CH₂]ₜ—N(R_{30c})—C(O)—N(R_{30b})R₃₀ₐ, —[CH₂]ₜ—S(O)ₚR₃₀ₐ (where p is 0, 1 or 2), —[CH₂]ₜ—SO₂N(R_{30b})R₃₀ₐ, or —[CH₂]ₜ—N(R_{30b})SO₂R₃₀ₐ; wherein
   t is 0, 1, 2 or 3;
   R₃₀ₐ is hydrogen or (1-4C)alkyl optionally substituted by halo, hydroxy, amino or cyano; and
   R_{30b} and R_{30c} are hydrogen or (1-2C)alkyl;
(iv) a group of the formula V or VI:

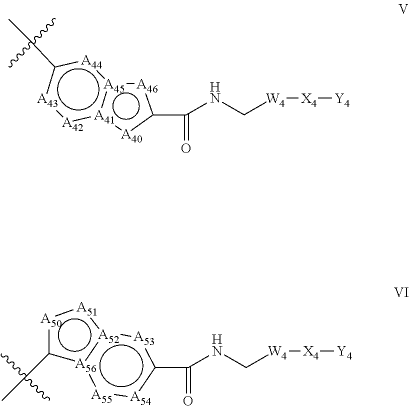

wherein:
   A₄₀ is selected from NH, NMe or O;
   A₄₂, A₄₃, A₄₄ and A₄₆ are each independently selected from CH, N or CR₂; A₄₁ and A₄₅ are each independently selected from C or N;
   with the proviso that:
      (i) only up to three of A₄₀, A₄₁, A₄₂, A₄₃, A₄₄, A₄₅ and A₄₆ are N;
      (ii) A₄₁ and A₄₅ cannot both be N;
      (iii) only one or two of A₄₀, A₄₂, A₄₃, A₄₄ and A₄₆ can be CR₂;
   A₅₁ is selected from NH, NMe, CH or CR₂;
   A₅₀, A₅₃, A₅₄ and A₅₅ are each independently selected from CH, N or CR₂;
   A₅₂ and A₅₆ are each independently selected from C or N;
   with the proviso that:
      (i) only up to three of A₅₀, A₅₁, A₅₂, A₅₃, A₅₄, A₅₅ and A₅₆ are N;
      (ii) A₅₂ and A₅₆ cannot both be N;
      (iii) only one or two of A₅₀, A₅₁, A₅₃, A₅₄ and A₅₅ can be CR₂;
   wherein R₂ is as defined above;
   W₄ is:

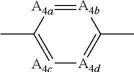

wherein A₄ₐ, A_{4b}, A_{4c} or A_{4d} are selected from CH, N or C—F, with the provisos that:
only one or two of A₄ₐ, A_{4b}, A_{4c} or A_{4d} can be N; and
only one or two of A₄ₐ, A_{4b}, A_{4c} or A_{4d} can be C—F;
X₄ is a linker group of the formula:

—[CH₂]ⱼ₁-L₄-[CH₂]ⱼ₂—
wherein
j1 and j2 are selected from 0 or 1;
L₄ is selected from —O—, —S—, —SO—, —SO₂—, —N(Rₓ₄ₐ)—, —C(O)—, —C(O)O—, —OC(O)—, —C(O)N(Rₓ₄ₐ)—, —N(Rₓ₄ₐ)C(O)—, —N(R_{x4b})C(O)N(Rₓ₄ₐ)—, —N(Rₓ₄ₐ)C(O)O—, —OC(O)N(Rₓ₄ₐ)—, —S(O)₂N(Rₓ₄ₐ), —N(Rₓ₄ₐ)SO₂—, or —C(O)N(Rₓ₄ₐ)SO₂—, or —SO₂N(Rₓ₄ₐ)C(O)—; and wherein Rₓ₄ₐ and R_{x4b} are each independently selected from hydrogen or (1-2C)alkyl;
Y₄ is selected from a carbocyclic, aryl, heteroaryl or heterocyclic ring which is optionally substituted by halo, nitro, cyano, R_{y4a}, —[CH₂]ᵤ—NR_{y4a}R_{y4b}, —[CH₂]—OR_{y4a}, —[CH₂]ᵤ—C(O)R_{y4a}, —[CH₂]ᵤ—C(O)OR_{y4a}, —[CH₂]ᵤ—OC(O)R_{y4a}, —[CH₂]ᵤ—C(O)N(R_{y4b})R_{y4a}, —[CH₂]ᵤ—N(R_{y4b})C(O)R_{y4a}, —[CH₂]ᵤ—N(R_{y4c})—C(O)—N(R_{y4b})R_{y4a}, —[CH₂]ᵤ—S(O)ₚR_{y4a} (where p is 0, 1 or 2), —[CH₂]ᵤ—SO₂N(R_{y4})R_{y4a}, or —[CH₂]ᵤ—N(R_{y4b})SO₂R_{y4a};
   wherein u is 0, 1, 2 or 3;
   R_{y4} is hydrogen or (1-4C)alkyl optionally substituted by halo, hydroxy, amino or cyano; and
   R_{y4b} and R_{y4c} are hydrogen or (1-2C)alkyl.

Particular compounds of the invention include, for example, compounds of Formula I or any sub-formula thereof, or pharmaceutically acceptable salts and/or solvates thereof, wherein, unless otherwise stated, R₁, and any associated substituent groups has any of the meanings defined hereinbefore or in any of paragraphs (1) to (33) hereinafter:—
(1) R₁ is selected from:
   (i) a group of the formula II:

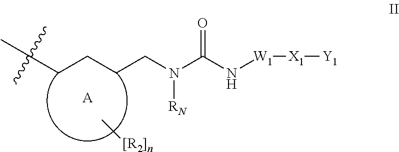

wherein:
Ring A is selected from phenyl or a 5- or 6-membered heteroaryl;
n is 0, 1 or 2;
each R₂ group, when present, is selected from: halo, nitro, cyano, R₂ₐ, —[CH₂]_{q}—NR₂ₐR_{2b}, —[CH₂]_{q}—OR₂ₐ, —[CH₂]_{q}—C(O)R₂ₐ, —[CH₂]_{q}—C(O)OR₂ₐ, —[CH₂]_{q}—OC(O)R₂ₐ, —[CH₂]_{q}—C(O)N(R_{2b})R₂ₐ, —[CH₂]_{q}—N(R_{2b})C(O)R₂ₐ, or —[CH₂]_{q}—S(O)ₚR₂ₐ (where p is 0, 1 or 2);
   wherein q is 0, 1 or 2;
   R₂ₐ is hydrogen or (1-4C)alkyl optionally substituted by halo, hydroxy, amino or cyano; and
   R_{2b} and R_{2c} are hydrogen or (1-2C)alkyl;
R_{N} is selected from hydrogen or methyl;
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, (1-2C)alkyl, (1-2C)haloalkyl, cyano or amino;
W₁ is:

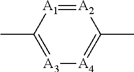

wherein A₁, A₂, A₃ or A₄ are selected from CH, N or C—F, with the provisos that:
only one or two of A₁, A₂, A₃ or A₄ can be N; and
only one or two of A₁, A₂, A₃ or A₄ can be C—F;
X₁ is a linker group of the formula:

—[CH₂]ₙ₁-L₁-[CH₂]ₙ₂—
wherein
n1 and n2 are selected from 0 or 1;
L₁ is selected from —O—, —S—, —SO—, —SO₂—, —N(Rₓ₁ₐ)—, —C(O)—, —C(O)O—, —OC(O)—, —C(O)N(Rₓ₁ₐ)—, —N(Rₓ₁ₐ)C(O)—, —N(R_{x1b})C(O)N(Rₓ₁ₐ)—, —N(Rₓ₁ₐ)C(O)O— or —OC(O)N(Rₓ₁ₐ)—; and wherein Rₓ₁ₐ and R_{x1b} are each independently selected from hydrogen or (1-2C)alkyl;
Y₁ is selected from a carbocyclic, aryl, heteroaryl or heterocyclic ring which is optionally substituted by halo, nitro, cyano, R_{y1a}, —[CH₂]ᵣ—NR_{y1a}R_{y1b}, —[CH₂]ᵣ—OR_{y1a}, —[CH₂]ᵣ—C(O)R_{y1a}, —[CH₂]ᵣ—C(O)OR_{y1a}, —[CH₂]ᵣ—OC(O)R_{y1a}, —[CH₂]ᵣ—C(O)N(R_{y1b})R_{y1a}, —[CH₂]ᵣ—N(R_{y1b})C(O)R_{y1a} or —[CH₂]ᵣ—S(O)ₚR_{y1a} (where p is 0, 1 or 2);
   wherein r is 0, 1 or 2;
   R_{y1} is hydrogen or (1-4C)alkyl optionally substituted by halo, hydroxy, amino or cyano; and
   R_{y1b} and R_{y1c} are hydrogen or (1-2C)alkyl;
(ii) a group of the formula III:

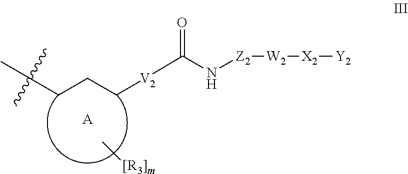

wherein:
Ring B is selected from phenyl or a 5- or 6-membered heteroaryl;
m is 0, 1 or 2;
each R₃ group, when present, is selected from: halo, nitro, cyano, R₃ₐ, —[CH₂]_{q1}—NR₃ₐR_{3b}, —[CH₂]_{q1}—OR₃ₐ, —[CH₂]_{q1}—C(O)R₃ₐ, —[CH₂]_{q1}—C(O)OR₃ₐ, —[CH₂]_{q1}—OC(O)R₃ₐ, —[CH₂]_{q1}—C(O)N(R_{3b})R₃ₐ, —[CH₂]_{q1}—N(R_{3b})C(O)R₃ₐ or —[CH₂]_{q1}—S(O)ₚR₃ₐ (where p is 0, 1 or 2);
   wherein q1 is 0, 1 or 2;
   R₃ₐ is hydrogen or (1-4C)alkyl optionally substituted by halo, hydroxy, amino or cyano; and
   R_{3b} and R_{3c} are hydrogen or (1-2C)alkyl;
V₂ is selected from —C(Rᵥ₂ₐR_{v2b})C(R_{v2c}R_{v2d})— or —C(Rᵥ₂ₐ)═C(R_{2c})—, wherein Rᵥ₂ₐ, R_{v2b}, R_{v2c} and R_{v2d} are each independently selected from hydrogen or methyl or Rᵥ₂ₐ and R_{v2c} are linked to form a cyclopropyl ring;
Z₂ is a 4C alkylene linker which is optionally substituted by one or more fluoro atoms;
W₂ is a carbocyclic, aryl, heteroaryl or heterocyclic ring which is optionally substituted by halo, nitro, cyano, R_{w2a}, —[CH₂]ₛ—NR_{w2a}R_{w2b}, —[CH₂]ₛ—OR_{w2a}, —[CH₂]ₛ—C(O)R_{w2a}, —[CH₂]ₛ—C(O)OR_{w2a}, —[CH₂]ₛ—OC(O)R_{w2a}, —[CH₂]ₛ—C(O)N(R_{w2b})R_{w2a}, —[CH₂]ₛ—N(R_{w2b})C(O)R_{w2a} or —[CH₂]ₛ—S(O)ₚR_{w2a} (where p is 0, 1 or 2); wherein
   s is 0, 1 or 2;
   R_{w2a} is hydrogen or (1-4C)alkyl optionally substituted by halo, hydroxy, amino or cyano; and
   R_{w2b} is hydrogen or (1-2C)alkyl;
X₂ is a linker group of the formula:

—[CH₂]ₘ₁-L₂-[CH₂]ₘ₂—
wherein
m1 and m2 are selected from 0 or 1;
L₂ is selected from —O—, —S—, —SO—, —SO₂—, —N(Rₓ₂ₐ)—, —C(O)—, —C(O)O—, —OC(O)—, —C(O)N(Rₓ₂ₐ)—, —N(Rₓ₂ₐ)C(O)—, —N(R_{x2b})C(O)N(Rₓ₂ₐ)—, —N(Rₓ₂ₐ)C(O)O— or —OC(O)N(Rₓ₂ₐ)—; and wherein Rₓ₂ₐ and R_{x2b} are each independently selected from hydrogen or (1-2C)alkyl;
Y₂ is selected from a carbocyclic, aryl, heteroaryl or heterocyclic ring which is optionally substituted by halo, nitro, cyano, R_{y2a}, —[CH₂]ₜ—NR_{y2a}R_{y2b}, —[CH₂]ₜ—OR_{y2a}, —[CH₂]ₜ—C(O)R_{y2a}, —[CH₂]ₜ—C(O)OR_{y2a}, —[CH₂]ₜ—OC(O)R_{y2a}, —[CH₂]ₜ—C(O)N(R_{y2b})R_{y2a}, —[CH₂]ₜ—N(R_{y2b})C(O)R_{y2a} or —[CH₂]ₜ—S(O)ₚR_{y2a} (where p is 0, 1 or 2);
   wherein t is 0, 1 or 2;
   R_{y2a} is hydrogen or (1-4C)alkyl optionally substituted by halo, hydroxy, amino or cyano; and
   R_{y2b} and R_{y2c} are hydrogen or (1-2C)alkyl;
(iii) a group of the formula IV:

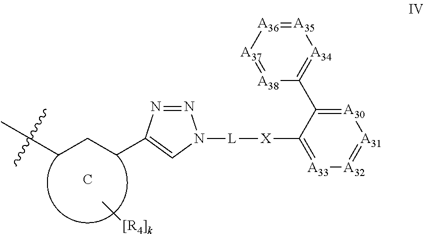

wherein:
Ring C is selected from phenyl, a 5- or 6-membered heteroaryl;
k is 0, 1 or 2;
each R₄ group, when present, is selected from: halo, nitro, cyano, R₄ₐ, —[CH₂]_{q2}—NR₄ₐR_{4b}, —[CH₂]_{q2}—OR₄ₐ, —[CH₂]_{q2}—C(O)R₄ₐ, —[CH₂]_{q2}—C(O)OR₄ₐ, —[CH₂]_{q2}—OC(O)R₄ₐ, —[CH₂]_{q2}—C(O)N(R_{4b})R₄ₐ or —[CH₂]_{q2}—N(R_{4b})C(O)R₄ₐ, —[CH₂]_{q2}—S(O)ₚR₄ₐ (where p is 0, 1 or 2);
   wherein q2 is 0, 1, 2 or 3;
   R₄ₐ is hydrogen or (1-4C)alkyl optionally substituted by halo, hydroxy, amino or cyano; and
   R_{4b} and R₄ₑ are hydrogen or (1-2C)alkyl;
L is a 6C alkylene linker which is optionally substituted by one or more fluoro atoms;
X is selected from —O—, —CRₓₐR_{xb}—, —S—, —SO—, —SO₂—, —N(Rₓₐ)—, —C(O)—; and wherein Rₓₐ and R_{xb} are each independently selected from hydrogen or methyl;
A₃₀ and A₃₁ are selected from CH, N or C—F;
A₃₂ and A₃₃ are selected from CH or N;
with the proviso that only one or two of A₃₀, A₃₁, A₃₂ and A₃₃ can be N;
A₃₄ and A₃₅ are selected from CH, N or C—R₃₀;
A₃₆, A₃₇ and A₃₈ are selected from CH or N;
with the proviso that only one or two of A₃₄, A₃₅, A₃₆, A₃₇ and A₃₈ can be N;
and wherein R₃₀ is selected from halo, nitro, cyano, R₃₀ₐ, —[CH₂]ₜ—NR₃₀ₐR_{30b}, —[CH₂]ₜ—OR₃₀ₐ, —[CH₂]ₜ—C(O)R₃₀ₐ, —[CH₂]ₜ—C(O)OR₃₀ₐ, —[CH₂]ₜ—OC(O)R₃₀ₐ, —[CH₂]ₜ—C(O)N(R_{30b})R₃₀ₐ, —[CH₂]ₜ—N(R_{30b})C(O)R₃₀ₐ or —[CH₂]ₜ—S(O)ₚR₃₀ₐ (where p is 0, 1 or 2); wherein
   t is 0, 1 or 2;
   R₃₀ₐ is hydrogen or (1-4C)alkyl optionally substituted by halo, hydroxy, amino or cyano; and
   R_{30b} and R_{30c} are hydrogen or (1-2C)alkyl;
(iv) a group of the formula V or VI:

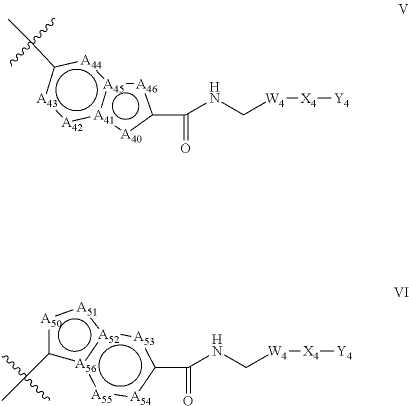

wherein:
   A₄₀ is selected from NH, NMe or O;
   A₄₂, A₄₃, A₄₄ and A₄₆ are each independently selected from CH, N or CR₂;
   A₄₁ and A₄₅ are each independently selected from C or N;
   with the proviso that:
   (i) only up to three of A₄₀, A₄₁, A₄₂, A₄₃, A₄₄, A₄₅ and A₄₆ are N;
   (ii) A₄₁ and A₄₅ cannot both be N;
   (iii) only one or two of A₄₀, A₄₂, A₄₃, A₄₄ and A₄₆ can be CR₂;
   A₅₁ is selected from NH, NMe, CH or CR₂;
   A₅₀, A₅₃, A₅₄ and A₅₅ are each independently selected from CH, N or CR₂;
   A₅₂ and A₅₆ are each independently selected from C or N;
   with the proviso that:
   (i) only up to three of A₅₀, A₅₁, A₅₂, A₅₃, A₅₄, A₅₅ and A₅₆ are N;
   (ii) A₅₂ and A₅₆ cannot both be N;
   (iii) only one or two of A₅₀, A₅₁, A₅₃, A₅₄ and A₅₅ can be C R₂;
   wherein R₂ is as defined above;
   W₄ is:

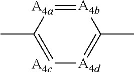

wherein A₄ₐ, A_{4b}, A_{4c} or A_{4d} are selected from CH, N or C—F, with the provisos that:
only one or two of A₄ₐ, A_{4b}, A_{4c} or A_{4d} can be N; and
only one or two of A₄ₐ, A_{4b}, A_{4c} or A_{4d} can be C—F;
X₄ is a linker group of the formula:

—[CH₂]ⱼ₁-L₄-[CH₂]ⱼ₂—
wherein
j1 and j2 are selected from 0 or 1;
L₄ is selected from —O—, —S—, —SO—, —SO₂—, —N(Rₓ₄ₐ)—, —C(O)—, —C(O)O—, —OC(O)—, —C(O)N(Rₓ₄ₐ)—, —N(Rₓ₄ₐ)C(O)—, —N(R_{x4b})C(O)N(Rₓ₄ₐ)—, —N(Rₓ₄ₐ)C(O)O— or —OC(O)N(Rₓ₄ₐ)—; and wherein Rₓ₄ₐ and R_{x4b} are each independently selected from hydrogen or (1-2C)alkyl;
Y₄ is selected from a carbocyclic, aryl, heteroaryl or heterocyclic ring which is optionally substituted by halo, nitro, cyano, R_{y4a}, —[CH₂]ᵤ—NR_{y4a}R_{y4b}, —[CH₂]ᵤ—OR_{y4a}, —[CH₂]ᵤ—C(O)R_{y4a}, —[CH₂]ᵤ—C(O)OR_{y4a}, —[CH₂]ᵤ—OC(O)R_{y4a}, —[CH₂]ᵤ—C(O)N(R_{y4b})R_{y4a}, —[CH₂]ᵤ—N(R_{y4b})C(O)R_{y4a} or —[CH₂]ᵤ—N(R_{y4c})—C(O)—N(R_{y4b})R_{y4a};
wherein u is 0, 1 or 2;
R_{y4} is hydrogen or (1-4C)alkyl optionally substituted by halo, hydroxy, amino or cyano; and
R_{y4b} and R_{y4c} are hydrogen or (1-2C)alkyl.
(2) R₁ is selected from:
   (i) a group of the formula II:

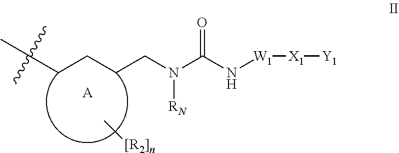

wherein:
Ring A is selected from phenyl or a 5- or 6-membered heteroaryl;
n is 0, 1 or 2;
each R₂ group, when present, is selected from: halo, nitro, cyano, R₂ₐ, —[CH₂]_{q}—NR₂ₐR_{2b}, —[CH₂]_{q}—OR₂ₐ, —[CH₂]_{q}—C(O)R₂ₐ, —[CH₂]_{q}—C(O)OR₂ₐ or —[CH₂]_{q}—OC(O)R₂ₐ;
   wherein q is 0, 1 or 2;
   R₂ₐ is hydrogen or (1-2C)alkyl optionally substituted by halo, hydroxy, amino or cyano; and
   R_{2b} is hydrogen or (1-2C)alkyl;
R_{N} is selected from hydrogen or methyl;
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, (1-2C)alkyl, (1-2C)haloalkyl, cyano or amino;
W₁ is:

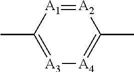

wherein A₁, A₂, A₃ or A₄ are selected from CH, N or C—F, with the provisos that:
only one or two of A₁, A₂, A₃ or A₄ can be N; and
only one or two of A₁, A₂, A₃ or A₄ can be C—F;
X₁ is a linker group of the formula:

—[CH₂]ₙ₁-L₁-[CH₂]ₙ₂—
wherein
n1 and n2 are selected from 0 or 1;
L₁ is selected from —O—, —S—, —SO—, —SO₂—, —N(Rₓ₁ₐ)—, —C(O)—, —C(O)O—, —OC(O)—, —C(O)N(Rₓ₁ₐ)— or —N(Rₓ₁ₐ)C(O)—; and wherein Rₓ₁ₐ and R_{x1b} are each independently selected from hydrogen or (1-2C)alkyl;
Y₁ is selected from a carbocyclic, aryl, heteroaryl or heterocyclic ring which is optionally substituted by halo, nitro, cyano, R_{y1a}, —[CH₂]ᵣ—NR_{y1a}R_{y1b}, —[CH₂]ᵣ—OR_{y1a}, —[CH₂]ᵣ—C(O)R_{y1a}, —[CH₂]ᵣ—C(O)OR_{y1a} or —[CH₂]ᵣ—OC(O)R_{y1a};
   wherein r is 0 or 1;
   R_{y1a} is hydrogen or (1-2C)alkyl optionally substituted by halo, hydroxy, amino or cyano; and
   R_{y1b} and R_{y1c} are hydrogen or (1-2C)alkyl;
(ii) a group of the formula III:

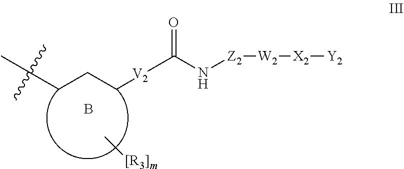

wherein:
Ring B is selected from phenyl or a 5- or 6-membered heteroaryl;
m is 0, 1 or 2;
each R₃ group, when present, is selected from: halo, nitro, cyano, R₃ₐ, —[CH₂]_{q1}—NR₃ₐR_{3b}, —[CH₂]_{q1}—OR₃ₐ, —[CH₂]_{q1}—C(O)R₃ₐ, —[CH₂]_{q1}—C(O)OR₃ₐ or —[CH₂]_{q1}—OC(O)R₃ₐ;
   wherein q1 is 0, 1 or 2;
   R₃ₐ is hydrogen or (1-2C)alkyl optionally substituted by halo, hydroxy, amino or cyano; and
   R_{3b} is hydrogen or (1-2C)alkyl;
V₂ is selected from —C(Rᵥ₂ₐR_{v2b})C(R_{2c}R_{v2d})— or —C(Rᵥ₂ₐ)═C(R_{v2c})—, wherein Rᵥ₂ₐ, R_{v2b}, R_{v2c} and R_{v2d} are each independently selected from hydrogen or methyl or Rᵥ₂ₐ and R_{v2c} are linked to form a cyclopropyl ring;
Z₂ is a 4C alkylene linker which is optionally substituted by one or more fluoro atoms;
W₂ is a carbocyclic, aryl, heteroaryl or heterocyclic ring which is optionally substituted by halo, nitro, cyano, R_{w2a}, —[CH₂]ₛ—NR_{w2a}R_{w2b}, —[CH₂]ₛ—OR_{w2a}, —[CH₂]ₛ—C(O)R_{w2a}, —[CH₂]ₛ—C(O)OR_{w2a} or —[CH₂]ₛ—OC(O)R_{w2a};
wherein
   s is 0, 1 or 2;
   R_{w2a} is hydrogen or (1-4C)alkyl optionally substituted by halo, hydroxy, amino or cyano; and
   R_{w2b} and R_{w2c} are hydrogen or (1-2C)alkyl;
X₂ is a linker group of the formula:

—[CH₂]ₘ₁-L₂-[CH₂]ₘ₂—
wherein
m1 and m2 are selected from 0 or 1;
L₂ is selected from —O—, —S—, —SO—, —SO₂—, —N(Rₓ₂ₐ)—, —C(O)—, —C(O)O—, —OC(O)—, —C(O)N(Rₓ₂ₐ)— or —N(Rₓ₂ₐ)C(O)—, and wherein Rₓ₂ₐ and R_{x2b} are each independently selected from hydrogen or (1-2C)alkyl;
Y₂ is selected from a carbocyclic, aryl, heteroaryl or heterocyclic ring which is optionally substituted by halo, nitro, cyano, R_{y2a}, —[CH₂]ₜ—NR_{y2a}R_{y2b}, —[CH₂]ₜ—OR_{y2a}, —[CH₂]ₜ—C(O)R_{y2a}, —[CH₂]ₜ—C(O)OR_{y2a} or —[CH₂]ₜ—OC(O)R_{y2a};
   wherein t is 0 or 1;
   R_{y2a} is hydrogen or (1-2C)alkyl optionally substituted by halo, hydroxy, amino or cyano; and
   R_{y2b} is hydrogen or (1-2C)alkyl;
(iii) a group of the formula IV:

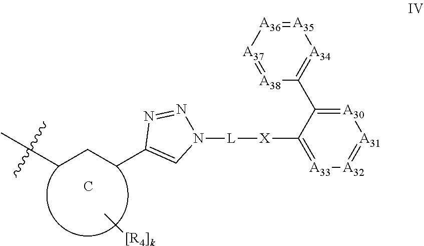

wherein:
Ring C is selected from phenyl, a 5- or 6-membered heteroaryl;
k is 0, 1 or 2;
each R₄ group, when present, is selected from: halo, nitro, cyano, R₄ₐ, —[CH₂]_{q2}—NR₄ₐR_{4b}, —[CH₂]_{q2}—OR₄ₐ, —[CH₂]_{q2}—C(O)R₄ₐ, —[CH₂]_{q2}—C(O)OR₄ₐ or —[CH₂]_{q2}—OC(O)R₄ₐ;
   wherein q2 is 0, 1, 2 or 3;
   R₄ₐ is hydrogen or (1-4C)alkyl optionally substituted by halo, hydroxy, amino or cyano; and
   R_{4b} is hydrogen or (1-2C)alkyl;
L is a 6C alkylene linker which is optionally substituted by one or more fluoro atoms;
X is selected from —O—, —CRₓₐR_{xb}—, —S—, —SO—, —SO₂—, —N(Rₓₐ)—, or —C(O)—; and wherein Rₓₐ and R_{xb} are each independently selected from hydrogen or methyl;
A₃₀ and A₃₁ are selected from CH, N or C—F;
A₃₂ and A₃₃ are selected from CH or N;
with the proviso that only one or two of A₃₀, A₃₁, A₃₂ and A₃₃ can be N;
A₃₄ and A₃₅ are selected from CH, N or C—R₃₀;
A₃₆, A₃₇ and A₃₈ are selected from CH or N;
with the proviso that only one or two of A₃₄, A₃₅, A₃₆, A₃₇ and A₃₈ can be N;
and wherein R₃₀ is selected from halo, nitro, cyano, R₃₀ₐ, —[CH₂]ₜ—NR₃₀ₐR_{30b}, —[CH₂]ₜ—OR₃₀ₐ, —[CH₂]ₜ—C(O)R₃₀ₐ, —[CH₂]ₜ—C(O)OR₃₀ₐ or —[CH₂]ₜ—OC(O)R₃₀ₐ; wherein
   t is 0, 1 or 2;
   R₃₀ₐ is hydrogen or (1-4C)alkyl optionally substituted by halo, hydroxy, amino or cyano; and
   R_{30b} and R_{30c} are hydrogen or (1-2C)alkyl;
(iv) a group of the formula V or VI:

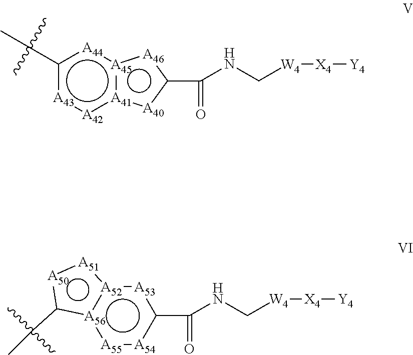

wherein:
   A₄₀ is selected from NH, NMe or O;
   A₄₂, A₄₃, A₄₄ and A₄₆ are each independently selected from CH, N or CR₂;
   A₄₁ and A₄₅ are each independently selected from C or N;
   with the proviso that:
      (i) only up to three of A₄₀, A₄₁, A₄₂, A₄₃, A₄₄, A₄₅ and A₄₆ are N;
      (ii) A₄₁ and A₄₅ cannot both be N;
      (iii) only one or two of A₄₀, A₄₂, A₄₃, A₄₄ and A₄₆ can be CR₂;
   A₅₁ is selected from NH, NMe, CH or CR₂;
   A₅₀, A₅₃, A₅₄ and A₅₅ are each independently selected from CH, N or CR₂;
   A₅₂ and A₅₆ are each independently selected from C or N;
   with the proviso that:
      (i) only up to three of A₅₀, A₅₁, A₅₂, A₅₃, A₅₄, A₅₅ and A₅₆ are N;
      (ii) A₅₂ and A₅₆ cannot both be N;
      (iii) only one or two of A₅₀, A₅₁, A₅₃, A₅₄ and A₅₅ can be CR₂;
   wherein R₂ is as defined above;
   W₄ is:

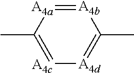

wherein A₄ₐ, A_{4b}, A_{4c} or A_{4d} are selected from CH, N or C—F, with the provisos that:
only one or two of A₄ₐ, A_{4b}, A_{4c} or A_{4d} can be N; and
only one or two of A₄ₐ, A_{4b}, A_{4c} or A_{4d} can be C—F;
X₄ is a linker group of the formula:

—[CH₂]ⱼ₁-L₄-[CH₂]ⱼ₂—
wherein
j1 and j2 are selected from 0 or 1;
L₄ is selected from —O—, —S—, —SO—, —SO₂—, —N(Rₓ₄ₐ)—, —C(O)—, —C(O)O—, —OC(O)—, —C(O)N(Rₓ₄ₐ)— or —N(Rₓ₄ₐ)C(O)—; and wherein Rₓ₄ₐ and R_{x4b} are each independently selected from hydrogen or (1-2C)alkyl;
Y₄ is selected from a carbocyclic, aryl, heteroaryl or heterocyclic ring which is optionally substituted by halo, nitro, cyano, R_{y4a}, —[CH₂]ᵤ—NR_{y4a}R_{y4b}, —[CH₂]ᵤ—OR_{y4a}, —[CH₂]ᵤ—C(O)R_{y4a}, —[CH₂]ᵤ—C(O)OR_{y4a} or —[CH₂]ᵤ—OC(O)R_{y4a},
   wherein u is 0 or 1;
   R_{y4a} is hydrogen or (1-2C)alkyl optionally substituted by halo, hydroxy, amino or cyano; and
   R_{y4b} and R_{y4c} are hydrogen or (1-2C)alkyl.
(3) R¹ is selected from:
   (i) a group of the formula II:

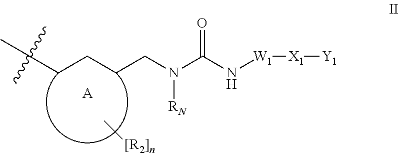

wherein:
Ring A is selected from phenyl or a 5- or 6-membered heteroaryl;
n is 0, 1 or 2;
each R₂ group, when present, is selected from: halo, nitro, cyano, R₂ₐ, —[CH₂]_{q}—NR₂ₐR_{2b}, —[CH₂]_{q}—OR₂ₐ, —[CH₂]_{q}—C(O)R₂ₐ;
   wherein q is 0 or 1;
   R₂ₐ is hydrogen or methyl; and
   R_{2b} is hydrogen or methyl;
R_{N} is selected from hydrogen or methyl;
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, methyl, halomethyl, cyano or amino;
W₁ is:

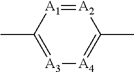

wherein A₁, A₂, A₃ or A₄ are selected from CH or C—F, with the proviso that:
only one or two of A₁, A₂, A₃ or A₄ can be C—F;
X₁ is a linker group of the formula:

—[CH₂]ₙ₁-L₁-[CH₂]ₙ₂—
wherein
n1 and n2 are selected from 0 or 1;
L₁ is selected from —O—, —S—, —SO—, —SO₂—, —N(Rₓ₁ₐ)— or —C(O)—; and wherein Rₓ₁ₐ is selected from hydrogen or methyl;
Y₁ is selected from a phenyl, heteroaryl or heterocyclic ring which is optionally substituted by halo, nitro, cyano, R_{y1a}, —NR_{y1a}R_{y1b}, —OR_{y1a} or —C(O)R_{y1a};
   R_{y1a} is hydrogen or methyl; and
   R_{y1b} is hydrogen or methyl;
(ii) a group of the formula III:

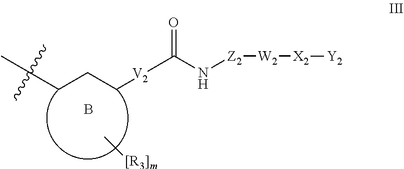

wherein:
Ring B is selected from phenyl or a 5- or 6-membered heteroaryl;
m is 0, 1 or 2;
each R₃ group, when present, is selected from: halo, nitro, cyano, R₃ₐ, —[CH₂]_{q1}—NR₃ₐR_{3b}, —[CH₂]_{q1}—OR₃ₐ or —[CH₂]_{q1}—C(O)R₃ₐ;
   wherein q1 is 0 or 1;
   R₃ₐ is hydrogen or methyl; and
   R_{3b} is hydrogen or methyl;
V₂ is selected from —C(Rᵥ₂ₐR_{v2b})C(R_{2c}R_{v2d})— or —C(Rᵥ₂ₐ)═C(R_{v2c})—, wherein Rᵥ₂ₐ, R_{v2b}, R_{v2c} and R_{v2d} are each independently selected from hydrogen or methyl or Rᵥ₂ₐ and R_{v2c} are linked to form a cyclopropyl ring;
Z₂ is a 4C alkylene linker which is optionally substituted by one or more fluoro atoms;
W₂ is a carbocyclic or heterocyclic ring which is optionally substituted by halo, nitro, cyano, R_{w2a}, —[CH₂]ₛ—NR_{w2a}R_{w2b}, —[CH₂]ₛ—OR_{w2a} or —[CH₂]ₛ—C(O)R_{w2a}; wherein
   s is 0 or 1;
   R_{w2a} is hydrogen or (1-2C)alkyl optionally substituted by halo, hydroxy, amino or cyano; and
   R_{w2b} and R_{w2c} are hydrogen or (1-2C)alkyl;
X₂ is a linker group of the formula:

—[CH₂]ₘ₁-L₂-[CH₂]ₘ₂—
wherein
m1 and m2 are selected from 0 or 1;
L₂ is selected from —O—, —S—, —SO—, —SO₂—, —N(Rₓ₂ₐ)— or —C(O)— and wherein Rₓ₂ₐ is selected from hydrogen or methyl;
Y₂ is selected from a phenyl, heteroaryl or heterocyclic ring which is optionally substituted by halo, nitro, cyano, R_{y2a}, NR_{y2a}R_{y2b}, OR_{y2a} or C(O)R_{y2a};
   R_{y2a} is hydrogen or methyl; and
   R_{y2b} is hydrogen or methyl;
(iii) a group of the formula IV:

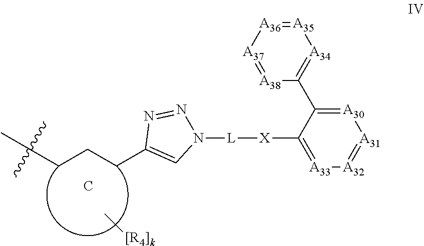

wherein:
Ring C is selected from phenyl, a 5- or 6-membered heteroaryl;
k is 0, 1 or 2;
each R₄ group, when present, is selected from: halo, nitro, cyano, R₄ₐ, —[CH₂]_{q2}—NR₄ₐR_{4b}, —[CH₂]_{q2}—OR₄ₐ, or —[CH₂]_{q2}—C(O)R₄ₐ;
   wherein q2 is 0 or 1;
   R₄ₐ is hydrogen or methyl; and
   R_{4b} is hydrogen or methyl;
L is a 6C alkylene linker which is optionally substituted by one or more fluoro atoms;
X is selected from —O—, —CRₓₐR_{xb}—, —S—, —SO—, —SO₂—, —N(Rₓₐ)—, or —C(O)—; and wherein Rₓₐ and R_{xb} are each independently selected from hydrogen or methyl;
A₃₀ and A₃₁ are selected from CH, N or C—F;
A₃₂ and A₃₃ are selected from CH or N;
with the proviso that only one or two of A₃₀, A₃₁, A₃₂ and A₃₃ can be N;
A₃₄ and A₃₅ are selected from CH, N or C—R₃₀;
A₃₆, A₃₇ and A₃₈ are selected from CH or N;
with the proviso that only one or two of A₃₄, A₃₅, A₃₆, A₃₇ and A₃₈ can be N;
and wherein R₃₀ is selected from halo, nitro, cyano, R₃₀ₐ, —NR₃₀ₐR_{30b}, —OR₃₀ₐ or —C(O)R₃₀ₐ; wherein
   R₃₀ₐ is hydrogen or methyl; and
   R_{30b} is hydrogen or methyl;
(iv) a group of the formula V or VI:

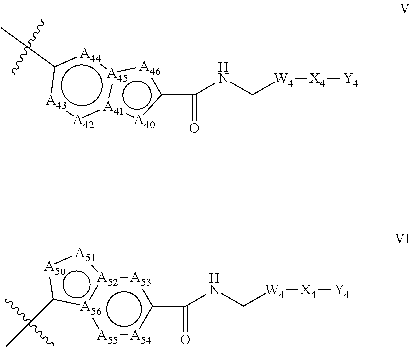

wherein:
   A₄₀ is selected from NH, NMe or O;
   A₄₂, A₄₃, A₄₄ and A₄₆ are each independently selected from CH, N or CR₂;
   A₄₁ and A₄₅ are each independently selected from C or N;
   with the proviso that:
      (i) only up to three of A₄₀, A₄₁, A₄₂, A₄₃, A₄₄, A₄₅ and A₄₆ are N;
      (ii) A₄₁ and A₄₅ cannot both be N;
      (iii) only one or two of A₄₀, A₄₂, A₄₃, A₄₄ and A₄₆ can be CR₂;
   A₅₁ is selected from NH, NMe, CH or CR₂;
   A₅₀, A₅₃, A₅₄ and A₅₅ are each independently selected from CH, N or CR₂;
   A₅₂ and A₅₆ are each independently selected from C or N;
   with the proviso that:
      (i) only up to three of A₅₀, A₅₁, A₅₂, A₅₃, A₅₄, A₅₅ and A₅₆ are N;
      (ii) A₅₂ and A₅₆ cannot both be N;
      (iii) only one or two of A₅₀, A₅₁, A₅₃, A₅₄ and A₅₅ can be CR₂;
   wherein R₂ is as defined above;
   W₄ is:

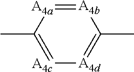

wherein A₄ₐ, A_{4b}, A_{4c} or A_{4d} are selected from CH or C—F, with the proviso that:
only one or two of A₄ₐ, A_{4b}, A_{4c} or A_{4d} can be C—F;
X₄ is a linker group of the formula:

—[CH₂]ⱼ₁-L₄-[CH₂]ⱼ₂—
wherein
j1 and j2 are selected from 0 or 1;
L₄ is selected from —O—, —S—, —SO—, —SO₂—, —N(Rₓ₄ₐ)— or —C(O)—, —C(O)O—, —OC(O)—, —C(O)N(Rₓ₄ₐ)— or —N(Rₓ₄ₐ)C(O)—; and wherein Rₓ₄ₐ and R_{x4b} are each independently selected from hydrogen or methyl;
Y₄ is selected from a phenyl, heteroaryl or heterocyclic ring which is optionally substituted by halo, nitro, cyano, R_{y4a}, —NR_{y4a}R_{y4b}, —OR_{y4a} or —C(O)R_{y4a};
   wherein
   R_{y4a} is hydrogen or methyl; and
   R_{y4b} is hydrogen or methyl.
(4) R¹ is selected from:
   (i) a group of the formula II:

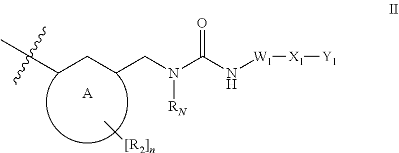

wherein:
Ring A is selected from phenyl or a 6-membered heteroaryl;
n is 0 or 1;
each R₂ group, when present, is selected from: halo, nitro, cyano, R₂ₐ, —NR₂ₐR_{2b}, —OR₂ₐ or —C(O)R₂ₐ;
   R₂ₐ is hydrogen or methyl; and
   R_{2b} is hydrogen or methyl;
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, methyl, halomethyl, cyano or amino;
W₁ is:

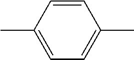

X₁ is a linker group of the formula:

—[CH₂]ₙ₁-L₁-[CH₂]ₙ₂—
wherein
n1 and n2 are selected from 0 or 1;
L₁ is selected from —O—, —S—, —SO₂— or —C(O)—;
Y₁ is selected from a phenyl, heteroaryl or heterocyclic ring which is optionally substituted by halo, nitro, cyano, R_{y1a} or —OR_{y1a};
   R_{y1a} is hydrogen or methyl; and
   R_{y1b} is hydrogen or methyl;
(ii) a group of the formula III:

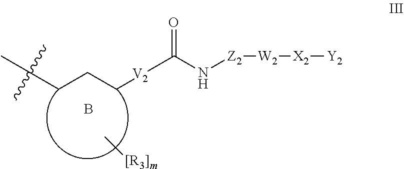

wherein:
Ring B is selected from phenyl or a 5- or 6-membered heteroaryl;
m is 0 or 1;
each R₃ group, when present, is selected from: halo, nitro, cyano, R₃ₐ, —NR₃ₐR_{3b}, —OR₃ₐ or —C(O)R₃ₐ;
   R₃ₐ is hydrogen or methyl; and
   R_{3b} is hydrogen or methyl;
V₂ is selected from —C(Rᵥ₂ₐR_{v2b})C(R_{v2c}R_{v2d})— or —C(Rᵥ₂ₐ)═C(R_{2c})—, wherein Rᵥ₂ₐ, R_{v2b}, R_{v2c} and R_{v2d} are each independently selected from hydrogen or methyl or Rᵥ₂ₐ and R_{v2c} are linked to form a cyclopropyl ring;
Z₂ is a 4C alkylene linker which is optionally substituted by one or more fluoro atoms;
W₂ is a carbocyclic or heterocyclic ring which is optionally substituted by halo, nitro, cyano, R_{w2a}, —NR_{w2a}R_{w2b}, —OR_{w2a} or —C(O)R_{w2a}; wherein
   R_{w2a} is hydrogen or methyl; and
   R_{w2b} and R_{w2e} are hydrogen or methyl;
X₂ is a linker group of the formula:

—[CH₂]ₘ₁-L₂-[CH₂]ₘ₂—
wherein
m1 and m2 are selected from 0 or 1;
L₂ is selected from —O—, —S—, —SO₂— or —C(O)—;
Y₂ is selected from a phenyl, heteroaryl or heterocyclic ring which is optionally substituted by halo, nitro, cyano, hydrogen, methyl or OR_{y2a};
   wherein R_{y2a} is hydrogen or methyl;
(iii) a group of the formula IV:

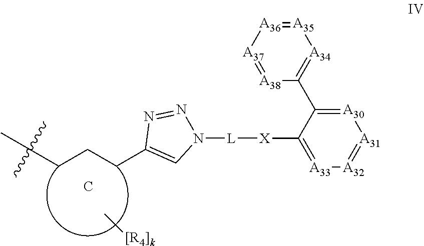

wherein:
Ring C is selected from phenyl or a 6-membered heteroaryl;
k is 0 or 1;
each R₄ group, when present, is selected from: halo, nitro, cyano, R₄ₐ, —NR₄ₐR_{4b}, —OR₄ₐ or —C(O)R₄ₐ;
   R₄ₐ is hydrogen or methyl; and
   R_{4b} is hydrogen or methyl;
L is a 6C alkylene linker which is optionally substituted by one or more fluoro atoms;
X is selected from —O—, —S—, —SO₂— or —C(O)—;
A₃₀ and A₃₁ are selected from CH or C—F;
A₃₂ and A₃₃ are CH;
A₃₄ and A₃₅ are selected from CH or C—R₃₀;
A₃₆, A₃₇ and A₃₈ are CH;
and wherein R₃₀ is selected from halo, nitro, cyano, hydrogen, methyl, —NR₃₀ₐR_{30b}, —OR₃₀ₐ or —C(O)R₃₀ₐ; wherein
   R₃₀ₐ is hydrogen or methyl; and
   R_{30b} is hydrogen or methyl;
(iv) a group of the formula V or VI:

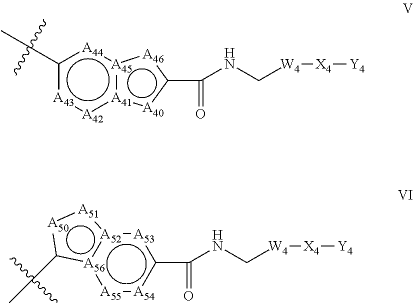

wherein:
   A₄₀ is selected from NH or O;
   A₄₂, A₄₃, A₄₄ and A₄₆ are each independently selected from CH or CR₂;
   A₄₁ and A₄₅ are each C;
   A₅₁ is selected from NH, NMe, CH or CR₂;
   A₅₀, A₅₃, A₅₄ and A₅₅ are each independently selected from CH or CR₂;
   A₅₂ and A₅₆ are each C;
   wherein R₂ is as defined above;
   W₄ is:

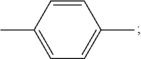

X₄ is a linker group of the formula:

-L₄-
wherein
L₄ is selected from —O—, —S—, —SO—, —SO₂—, —N(Rₓ₄ₐ)— or —C(O)—, —C(O)O—, —OC(O)—, —C(O)N(Rₓ₄ₐ)— or —N(Rₓ₄ₐ)C(O)—; and wherein Rₓ₄ₐ and R_{x4b} are each independently selected from hydrogen or methyl;
Y₄ is selected from a phenyl, heteroaryl or heterocyclic ring which is optionally substituted by halo, nitro, cyano or methyl;
(5) R¹ is selected from:
   (i) a group of the formula II:

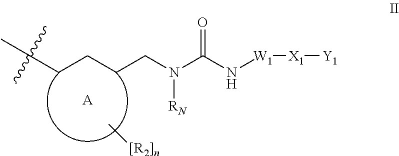

wherein:
Ring A is selected from phenyl or a 6-membered heteroaryl;
n is 0 or 1;
each R₂ group, when present, is selected from: halo or —OR₂ₐ;
   wherein R₂ₐ is hydrogen or methyl; and
R_{N} is hydrogen
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, methyl, halomethyl, cyano or amino;
W₁ is:

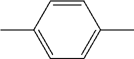

X₁ is a linker group of the formula:

—[CH₂]ₙ₁-L₁-[CH₂]ₙ₂—
wherein
n1 and n2 are selected from 0 or 1;
L₁ is selected from —O—, —S—, —SO₂— or —C(O)—;
Y₁ is selected from a phenyl, or heterocyclic ring which is optionally substituted by halo, nitro, cyano, hydrogen, methyl, or —OR_{y1a};
   R_{y1a} is hydrogen or methyl;
(ii) a group of the formula III:

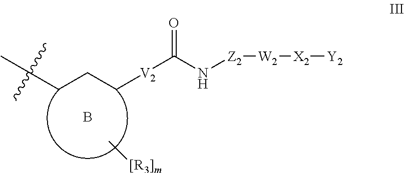

wherein:
Ring B is selected from phenyl or a 5- or 6-membered heteroaryl;
m is 0 or 1;
each R₃ group, when present, is selected from: halo or —OR₃ₐ;
   wherein R₃ₐ is hydrogen or methyl;
V₂ is selected from —C(Rᵥ₂ₐR_{v2b})C(R_{v2c}R_{v2d})— or —C(Rᵥ₂ₐ)═C(R_{v2c})—, wherein Rᵥ₂ₐ, R_{v2b}, R_{v2c} and R_{v2d} are each independently selected from hydrogen or methyl or Rᵥ₂ₐ and R_{v2c} are linked to form a cyclopropyl ring;
Z₂ is a 4C alkylene linker which is optionally substituted by one or more fluoro atoms;
W₂ is a heterocyclic ring which is optionally substituted by halo, or —OR_{w2a}; wherein
   R_{w2a} is hydrogen or methyl;
X₂ is a linker group of the formula:

-L₂-
wherein
L₂ is selected from —O—, —S—, —SO₂— or —C(O)—;
Y₂ is a phenyl or heterocyclic ring which is optionally substituted by halo, nitro, cyano, hydrogen, methyl or OR_{y2a};
   R_{y2a} is hydrogen or methyl;
(iii) a group of the formula IVa:

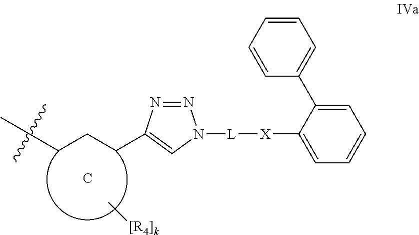

wherein:
Ring C is selected from phenyl or a 6-membered heteroaryl;
k is 0 or 1;
each R₄ group, when present, is selected from: halo, or —OR₄ₐ;
   wherein R₄ₐ is hydrogen or methyl;
L is a 6C alkylene linker which is optionally substituted by one or more fluoro atoms;
X is selected from —O—, —S—, —SO₂—, or —C(O)—;
(iv) a group of the formula Va or VIa:

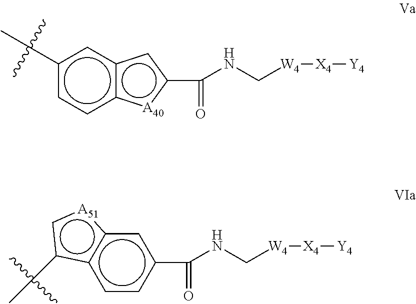

wherein:
   A₄₀ is selected from NH or O;
   A₅₁ is selected from NH, NMe, CH or CR₂;
   wherein R₂ is as defined above;
   W₄ is:

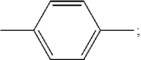

X₄ is a linker group of the formula:

-L₄-
wherein
L₄ is selected from —SO₂—, —C(O)N(Rₓ₄ₐ)— or —N(Rₓ₄ₐ)C(O)—; and wherein Rₓ₄ₐ is selected from hydrogen or methyl;
Y₄ is a phenyl or heterocyclic ring which is optionally substituted by halo, nitro, cyano or methyl.
(6) R¹ is selected from:
   (i) a group of the formula II:

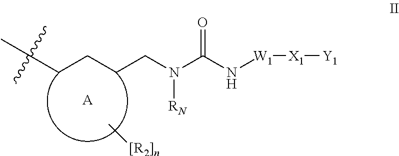

wherein:
Ring A is selected from phenyl or a 6-membered heteroaryl;
n is 0 or 1;
each R₂ group, when present, is selected from: halo or —OR₂ₐ;
wherein R₂ₐ is hydrogen or methyl; and
R_{N} is hydrogen
or a R₂ group and R_{N} are linked so as to form a 5-membered heterocyclic ring fused to Ring A, the fused 5-membered heterocyclic ring comprising one or two N atoms;
W₁ is:

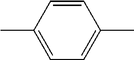

X₁ is a linker group of the formula:

—[CH₂]ₙ₁-L₁-[CH₂]ₙ₂—
wherein
n1 and n2 are selected from 0 or 1;
L₁ is selected from —SO₂—;
Y₁ is selected from a phenyl, or heterocyclic ring which is optionally substituted by halo, or —OR_{y1a};
   wherein R_{y1a} is hydrogen or methyl;
(ii) a group of the formula III:

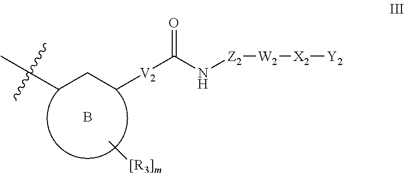

wherein:
Ring B is selected from phenyl or a 6-membered heteroaryl;
m is 0 or 1;
each R₃ group, when present, is selected from: halo, or —OR₃ₐ;
   wherein R₃ₐ is hydrogen or methyl;
V₂ is selected from —C(Rᵥ₂ₐR_{v2b})C(R_{v2c}R_{v2d})— or —C(Rᵥ₂ₐ)═C(R_{2c})—, wherein Rᵥ₂ₐ, R_{v2b}, R_{v2c} and R_{v2d} are each hydrogen, or Rᵥ₂ₐ and R_{v2c} are linked to form a cyclopropyl ring;
Z₂ is a 4C alkylene linker;
W₂ is a heterocyclic ring which is optionally substituted by halo, or —OR_{w2a}; wherein R_{w2a} is hydrogen or methyl;
X₂ is a linker group of the formula:

-L₂-
wherein
L₂ is selected from —C(O)—;
Y₂ is a phenyl ring which is optionally substituted by halo, or OR_{y2a}; wherein
   R_{y2a} is hydrogen or methyl;
(iii) a group of the formula IVa:

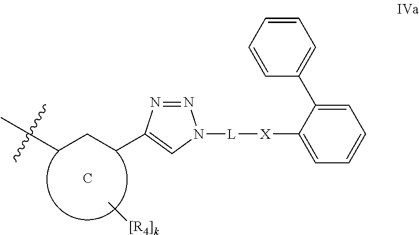

wherein:
Ring C is selected from phenyl or a 6-membered heteroaryl;
k is 0 or 1;
each R₄ group, when present, is selected from: halo, or —OR₄ₐ;
   wherein R₄ₐ is hydrogen or methyl;
L is a 6C alkylene linker;
X is —O—;
(iv) a group of the formula Va or VIa:

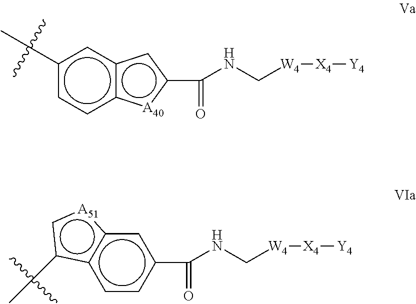

wherein:
   A₄₀ is selected from NH or O;
   A₅₁ is selected from NH, NMe, CH or CR₂;
   wherein R₂ is as defined above
   W₄ is:

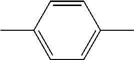

X₄ is a linker group of the formula:

-L₄-
wherein
L₄ is selected from —SO₂— or —C(O)N(Rₓ₄ₐ)—; and wherein Rₓ₄ₐ is independently selected from hydrogen or methyl;
Y₄ is a 5 or 6-membered heterocyclic ring.
(7) R¹ is selected from:
   (i) a group of the formula IIa or IIb:

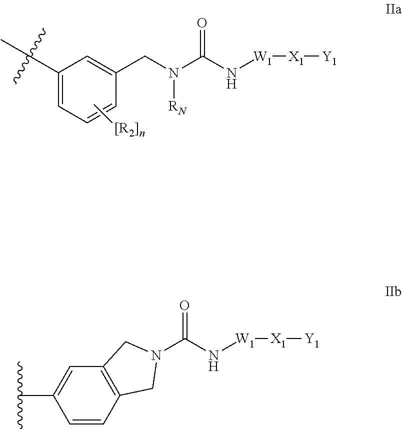

wherein:
Ring A is selected from phenyl or a 6-membered heteroaryl;
n is 0 or 1;
each R₂ group, when present, is selected from: halo or —OR₂ₐ;
wherein R₂ₐ is hydrogen or methyl; and
R_{N} is hydrogen
or a R₂ group and R_{N} are linked so as to form a 5-membered heterocyclic ring fused to Ring A, the fused 5-membered heterocyclic ring comprising one or two N atoms;
W₁ is:

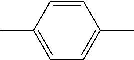

X₁ is a linker group of the formula:

—[CH₂]ₙ₁-L₁-[CH₂]ₙ₂—
wherein
n1 and n2 are selected from 0 or 1;
L₁ is selected from —SO₂—,
Y₁ is selected from a phenyl, or heterocyclic ring which is optionally substituted by halo, or —OR_{y1a};
   wherein R_{y1a} is hydrogen or methyl;
(ii) a group of the formula IIIa:

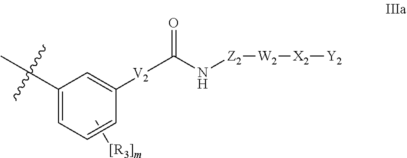

wherein:
m is 0 or 1;
each R₃ group, when present, is selected from: halo, or —OR₃ₐ;
   wherein R₃ₐ is hydrogen or methyl;
V₂ is selected from —C(Rᵥ₂ₐR_{v2b})C(R_{v2c}R_{v2d})— or —C(Rᵥ₂ₐ)═C(R_{2c})—, wherein Rᵥ₂ₐ, R_{v2b}, R_{v2c} and R_{v2d} are each hydrogen, or Rᵥ₂ₐ and R_{v2c} are linked to form a cyclopropyl ring;
Z₂ is a 4C alkylene linker;
W₂ is a heterocyclic ring which is optionally substituted by halo, or —OR_{w2a}; wherein R_{w2a} is hydrogen or methyl;
   X₂ is a linker group of the formula:

-L₂-
wherein
L₂ is selected from —C(O)—;
Y₂ is a phenyl ring which is optionally substituted by halo, or OR_{y2a}; wherein
   R_{y2a} is hydrogen or methyl;
(iii) a group of the formula IVb:

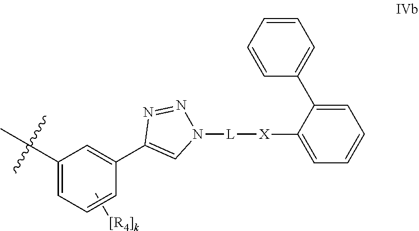

wherein:
k is 0 or 1;
each R₄ group, when present, is selected from: halo, or —OR₄ₐ;
   wherein R₄ₐ is hydrogen or methyl;
L is a 6C alkylene linker;
X is —O—;
(iv) a group of the formula Vb, Vc, VIb or VIc:

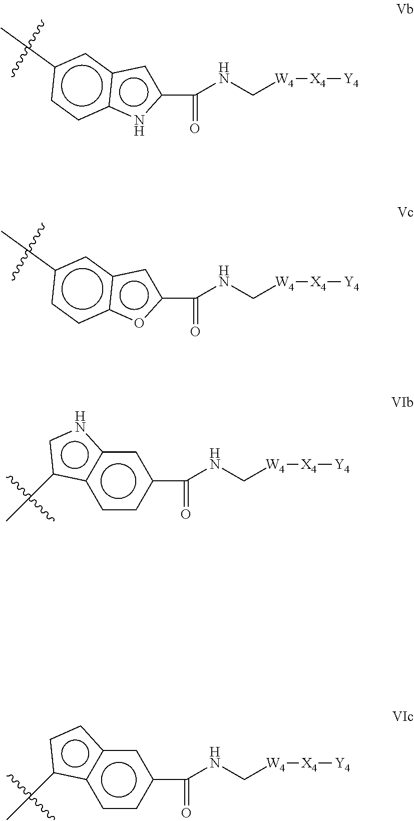

wherein:
   W₄ is:

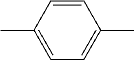

X₄ is a linker group of the formula:

-L₄-
wherein
L₄ is selected from —SO₂— or —C(O)N(H)—;
Y₄ is a 5 or 6 membered heterocyclic ring.

Suitably, a heteroaryl or heterocyclyl group as defined herein is a monocyclic 5- or 6-membered heteroaryl or 5- or 6-membered heterocyclyl group comprising one, two or three heteroatoms selected from N, O or S.

Suitably, a heteroaryl is a 5- or 6-membered heteroaryl ring comprising one, two or three heteroatoms selected from N, O or S.

Suitably, a heterocyclyl group is a 5-, 6-membered heterocyclyl ring comprising one, two or three heteroatoms selected from N, O or S; or is a 8, 9, or 10-membered spiro-fused heterocyclylic ring system comprising one, two or three heteroatoms selected from N, O or S.

Most suitably, a heterocyclyl group is a 5- or 6-membered ring comprising one, two or three heteroatoms selected from N, O or S [e.g. morpholinyl (e.g. 4-morpholinyl), pyridinyl, piperazinyl, or pyrrolidinonyl]. Suitably, a heterocyclyl group is a 8, 9, or 10-membered spiro-fused heterocyclylic ring system comprising one, two or three heteroatoms selected from N, O or S.

Suitably, R₁ is as defined in any one of paragraphs (1) to (7). More suitably, R₁ is as defined in any one of paragraphs (4) to (7). Most suitably, R₁ is as defined in paragraph (6) or (7).

Suitably, R₁ is selected from any one of formula II, III, IV, V, VI and any associated sub formulae defined herein, wherein any associated sub groups are as defined in any one of paragraphs (1) to (7). More suitably, the associated sub groups are as defined in any one of paragraphs (4) to (7). Most suitably, the associated sub groups are as defined in paragraph (6) or (7).

In a particular group of compounds of the invention, R¹ is a group of the formula II, below:

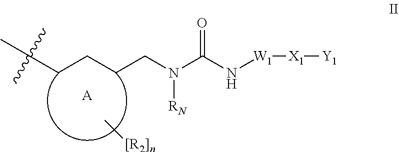

wherein Ring A, n, R₂, R_{N}, W₁, X₁ and Y₁ are as defined herein.

Suitably, Ring A, n, R₂, R_{N}, W₁, X₁ and Y₁ are as defined in any one of paragraphs (1) to (7). More suitably, Ring A, n, R₂, R_{N}, W₁, X₁ and Y₁ are as defined in any one of paragraphs (4) to (7). Most suitably, Ring A, n, R₂, R_{N}, W₁, X₁ and Y₁ are as defined in paragraph (6) or (7).

In a particular group of compounds of the invention, R¹ is a group of the formula IIa or IIb, below:

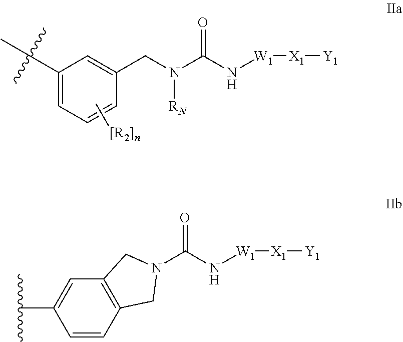

wherein n, R₂, R_{N}, W₁, X₁ and Y₁ are as defined herein.

Suitably, n, R₂, R_{N}, W₁, X₁ and Y₁ are as defined in any one of paragraphs (1) to (7).

More suitably, n, R₂, R_{N}, W₁, X₁ and Y₁ are as defined in any one of paragraphs (4) to (7).

Most suitably, n, R₂, R_{N}, W₁, X₁ and Y₁ are as defined in paragraph (6) or (7).

In a particular group of compounds of the invention, R¹ is a group of the formula III, below:

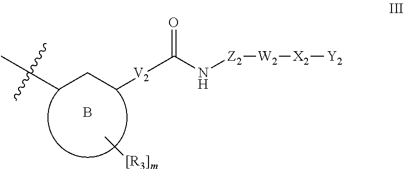

wherein Ring B, R₃, m, V₂, Z_{z}, W₂, X₂ and Y₂ are as defined herein.

Suitably, Ring B, m, R₃, V₂, Z_{z}, W₂, X₂ and Y₂ are as defined in any one of paragraphs (1) to (7). More suitably, Ring B, m, R₃, V₂, Z_{z}, W₂, X₂ and Y₂ are as defined in any one of paragraphs (4) to (7). Most suitably, Ring B, m, R₃, V₂, Z_{z}, W₂, X₂ and Y₂ are as defined in paragraph (6) or (7).

In a particular group of compounds of the invention, R¹ is a group of the formula IIIa, below:

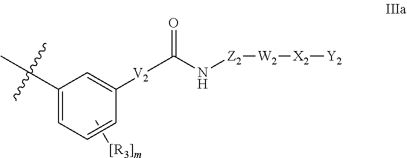

wherein Ring B, R₃, m, V₂, Z_{z}, W₂, X₂ and Y₂ are as defined herein.

Suitably, m, R₃, V₂, Z_{z}, W₂, X₂ and Y₂ are as defined in any one of paragraphs (1) to (7). More suitably, m, R₃, V₂, Z_{z}, W₂, X₂ and Y₂ are as defined in any one of paragraphs (4) to (7). Most suitably, m, R₃, V₂, Z_{z}, W₂, X₂ and Y₂ are as defined in paragraph (6) or (7).

In a particular group of compounds of the invention, R¹ is a group of the formula IV, below:

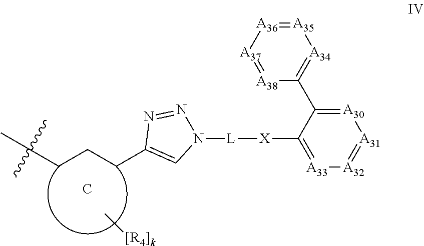

wherein Ring C, R₄, k, L, X, A₃₃, A₃₄, A₃₅, A₃₆, A₃₇ and A₃₈ are as defined herein.

Suitably, Ring C, R₄, k, L, X, A₃₃, A₃₄, A₃₅, A₃₆, A₃₇ and A₃₈ are as defined in any one of paragraphs (1) to (7). More suitably, Ring C, R₄, k, L, X, A₃₃, A₃₄, A₃₅, A₃₆, A₃₇ and A₃₈ are as defined in any one of paragraphs (4) to (7). Most suitably, Ring C, R₄, k, L, X, A₃₃, A₃₄, A₃₅, A₃₆, A₃₇ and A₃₈ are as defined in paragraph (6) or (7).

In a particular group of compounds of the invention, R¹ is a group of the formula IVa, below:

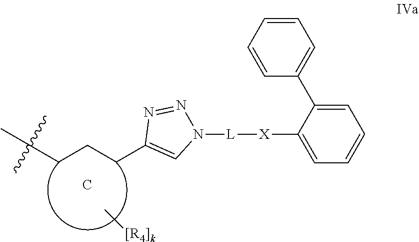

wherein Ring C, R₄, k, L and X are as defined herein.

Suitably, Ring C, R₄, k, L and X are as defined in any one of paragraphs (1) to (7).

More suitably, Ring C, R₄, k, L and X are as defined in any one of paragraphs (4) to (7). Most suitably, Ring C, R₄, k, L and X are as defined in paragraph (6) or (7).

In a particular group of compounds of the invention, R¹ is a group of the formula V or VI, below:

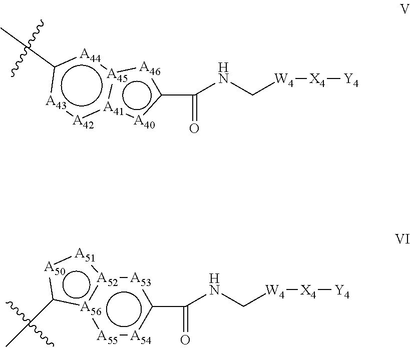

wherein A₄₃, A₅₄, A₅₅, A₄₆, A₄₇, A₅₀, A₅₁, A₅₂, A₅₃, A₅₄, A₅₅, A₅₆, W₄, X₄ and Y₄ are as defined herein.

Suitably, A₄₃, A₅₄, A₅₅, A₄₆, A₄₇, A₅₀, A₅₁, A₅₂, A₅₃, A₅₄, A₅₅, A₅₆, W₄, X₄ and Y₄ are as defined in any one of paragraphs (1) to (7). More suitably, A₄₃, A₅₄, A₅₅, A₄₆, A₄₇, A₅₀, A₅₁, A₅₂, A₅₃, A₅₄, A₅₅, A₅₆, W₄, X₄ and Y₄ are as defined in any one of paragraphs (4) to (7). Most suitably, A₄₃, A₅₄, A₅₅, A₄₆, A₄₇, A₅₀, A₅₁, A₅₂, A₅₃, A₅₄, A₅₅, A₅₆, W₄, X₄ and Y₄ are as defined in paragraph (6) or (7).

In a particular group of compounds of the invention, R¹ is a group of the formula Va or VIa, below:

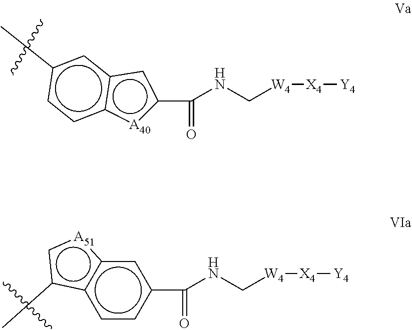

wherein A₄₀, A₅₁, W₄, X₄ and Y₄ are as defined herein.

Suitably, A₄₀, A₅₁, W₄, X₄ and Y₄ are as defined in any one of paragraphs (1) to (7).

More suitably, A₄₀, A₅₁, W₄, X₄ and Y₄ are as defined in any one of paragraphs (4) to (7). Most suitably, A₄₀, A₅₁, W₄, X₄ and Y₄ are as defined in paragraph (6) or (7).

Particular compounds of the present invention include any of the compounds exemplified in the present application, or a pharmaceutically acceptable salt or solvate thereof, and, in particular, any of the following:
6-(2-fluoro-5-((3-(4-(piperidin-1-ylsulfonyl)phenyl)ureido)methyl)phenyl)-4-(trioxidaneylthio)naphthalene-2-sulfonic acid;
(E)-6-(5-(3-((4-(1-benzoylpiperidin-4-yl)butyl)amino)-3-oxoprop-1-en-1-yl)-2-fluorophenyl)-4-(trioxidaneylthio)naphthalene-2-sulfonic acid;
6-(2-fluoro-5-((3-(4-(phenylsulfonyl)phenyl)ureido)methyl)phenyl)-4-(trioxidaneylthio) naphthalene-2-sulfonic acid;
6-(3-(1-(6-([1,1′-biphenyl]-2-yloxy)hexyl)-1H-1,2,3-triazol-4-yl)phenyl)-4-(trioxidaneylthio) naphthalene-2-sulfonic acid;
6-(3-((3-(4-(piperidin-1-ylsulfonyl)phenyl)ureido)methyl)phenyl)-4-(trioxidaneylthio) naphthalene-2-sulfonic acid;
6-(4-fluoro-3-((3-(4-(piperidin-1-ylsulfonyl)phenyl)ureido)methyl)phenyl)-4-(trioxidaneylthio) naphthalene-2-sulfonic acid;
6-(2-methoxy-5-((3-(4-(piperidin-1-ylsulfonyl)phenyl)ureido)methyl)phenyl)-4-(trioxidaneylthio)naphthalene-2-sulfonic acid;
6-(5-((3-(4-((8-oxa-3-azabicyclo[3.2.1]octan-3-yl)sulfonyl)phenyl)ureido)methyl)-2-fluorophenyl)-4-(trioxidaneylthio)naphthalene-2-sulfonic acid;
6-(2-((4-(piperidin-1-ylsulfonyl)benzyl)carbamoyl)-1H-indol-5-yl)-4-(trioxidaneylthio) naphthalene-2-sulfonic acid;
(S)-6-(2-((4-(((Tetrahydrofuran-3-yl)methyl)carbamoyl)phenyl)carbamoyl)isoindolin-5-yl)-4-(trioxidaneylthio)naphthalene-2-sulfonic acid.

The various functional groups and substituents making up the compounds of the formula I are typically chosen such that the molecular weight of the compound of the formula I does not exceed 1000. More usually, the molecular weight of the compound will be less than 900, for example less than 800, or less than 700, or less than 650, or less than 600. More preferably, the molecular weight is less than 550 and, for example, is 500 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn-Ingold-Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For example, H may be in any isotopic form, including ¹H, ²H(D), and ³H (T); C may be in any isotopic form, including ¹²C, ¹³C, and ¹⁴C; and O may be in any isotopic form, including ¹⁶O and ¹⁸O; and the like.

It is also to be understood that certain compounds of the formula I may exist in solvated as well as unsolvated forms such as, for example, hydrated forms. It is to be understood that the invention encompasses all such solvated forms that possess antiproliferative activity.

It is also to be understood that certain compounds of the formula I may exhibit polymorphism, and that the invention encompasses all such forms that possess antiproliferative activity.

Compounds of the formula I may exist in a number of different tautomeric forms and references to compounds of the formula I include all such forms. For the avoidance of doubt, where a compound can exist in one of several tautomeric forms, and only one is specifically described or shown, all others are nevertheless embraced by formula I. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

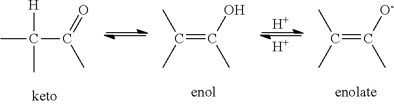

Compounds of the formula I containing an amine function may also form N-oxides. A reference herein to a compound of the formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of formula I may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula I and in-vivo cleavable amide derivatives that may be formed at a carboxy group or an amino group in a compound of the formula I.

Accordingly, the present invention includes those compounds of the formula I as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula I may be a synthetically-produced compound or a metabolically-produced compound.

A suitable pharmaceutically acceptable pro-drug of a compound of the formula I is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula I that possesses a carboxy group is, for example, an in vivo cleavable ester thereof. An in vivo cleavable ester of a compound of the formula I containing a carboxy group is, for example, a pharmaceutically acceptable ester which is cleaved in the human or animal body to produce the parent acid. Suitable pharmaceutically acceptable esters for carboxy include C₁₋₆alkyl esters such as methyl, ethyl and tert-butyl, C₁₋₆alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically acceptable pro-drug of a compound of the formula I that possesses a hydroxy group is, for example, an in vivo cleavable ester or ether thereof.

An in vivo cleavable ester or ether of a compound of the formula I containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N—(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula I that possesses a carboxy group is, for example, an in vivo cleavable amide thereof, for example an amide formed with an amine such as ammonia, a C₁₋₄alkylamine such as methylamine, a (C₁₋₄alkyl)₂amine such as dimethylamine, N-ethyl-N-methylamine or diethylamine, a C₁₋₄ alkoxy-C₂₋₄alkylamine such as 2-methoxyethylamine, a phenyl-C₁₋₄alkylamine such as benzylamine and amino acids such as glycine or an ester thereof.

A suitable pharmaceutically acceptable pro-drug of a compound of the formula I that possesses an amino group is, for example, an in vivo cleavable amide derivative thereof.

Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the formula I may be exerted in part by one or more metabolites that are formed within the human or animal body after administration of a compound of the formula I. As stated hereinbefore, the in vivo effects of a compound of the formula I may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide.

Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of formula I will vary depending on the nature of R₁ and any substituent groups associated therewith. Suitable processes for their preparation are described further in the accompanying Examples.

Once a compound of formula I has been synthesised by any one of the processes defined herein, the processes may then further comprise the additional steps of:
(i) removing any protecting groups present;
(ii) converting the compound formula I into another compound of formula I;
(iii) forming a pharmaceutically acceptable salt, hydrate or solvate thereof; and/or
(iv) forming a prodrug thereof.

An example of (ii) above is when a compound of formula I is synthesised and then one or more of the groups associated with R₁ may be further reacted to change the nature of the group and provide an alternative compound of formula I.

The resultant compounds of formula I can be isolated and purified using techniques well known in the art.

### Biological Activity

The enzyme and in-vitro cell-based assays described in accompanying Example section, or elsewhere in the literature, may be used to measure the pharmacological effects of the compounds of the present invention.

Although the pharmacological properties of the compounds of formula I vary with structural change, as expected, the compounds of the invention were found to be active in these enzyme assays.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier. For example, solid oral forms may contain, together with the active compound, diluents, such as, for example, lactose, dextrose, saccharose, cellulose, corn starch or potato starch; lubricants, such as, for example, silica, talc, stearic acid, magnesium or calcium stearate, and/or polyethylene glycols; binding agents; such as, for example, starches, arabic gums, gelatin, methylcellulose, carboxymethylcellulose or polyvinyl pyrrolidone; disaggregating agents, such as, for example, starch, alginic acid, alginates or sodium starch glycolate; effervescing mixtures; dyestuffs; sweeteners; wetting agents, such as, for example, lecithin, polysorbates, laurylsulphates; and, in general, non toxic and pharmacologically inactive substances used in pharmaceutical formulations. Such pharmaceutical compositions may be manufactured in by conventional methods known in the art, such as, for example, by mixing, granulating, tableting, sugar coating, or film coating processes.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing). Suitably, oral or parenteral administration is preferred. Most suitably, oral administration is preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the condition, the age and sex of the animal or patient and the route of administration, according to well known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as modulators or suppressors of eNAMPT activity.

The present invention therefore provides a method of suppressing eNAMPT activity in vitro or in vivo, said method comprising administering an effective amount of a compound, or a pharmaceutically acceptable salt thereof, or a pharmaceutical composition as defined herein.

The present invention also provides a method of treating a disease or disorder in which eNAMPT activity is implicated in a patient in need of such treatment, said method comprising administering to said patient a therapeutically effective amount of a compound, or a pharmaceutically acceptable salt thereof, or a pharmaceutical composition as defined herein.

The present invention provides a compound, or a pharmaceutically acceptable salt thereof, as defined herein for use in the treatment of a disease or disorder in which eNAMPT activity is implicated. Suitably, the disease or disorder is any of those listed herein.

In another aspect, the present invention provides a compound as defined herein, or a pharmaceutically acceptable salt or solvate thereof, for use in the treatment of a disease or condition in which the suppression of eNAMPT activity is beneficial. Suitably, a compound as defined herein, or a pharmaceutically acceptable salt or solvate thereof, is for use in the treatment of a disease or condition in which the suppression of monomeric eNAMPT activity is beneficial.

In another aspect, the present invention provides a compound as defined herein, or a pharmaceutically acceptable salt or solvate thereof, for use in the treatment of:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH));
(vi) inflammatory skin conditions (e.g. psoriasis);
(vii) lung conditions (Acute Lung Injury (ALI), Ventilator Induced Lung Injury (VILI), Radiation-Induced Lung Injury (RILI), pulmonary fibrosis (e.g. idiopathic pulmonary fibrosis));
(viii) arthritis (e.g. osteoarthritis or rheumatoid arthritis);
(ix) kidney disease (e.g. chronic kidney disease); or
(x) sepsis.

In another aspect, the present invention provides a compound as defined herein, or a pharmaceutically acceptable salt or solvate thereof, for use in the treatment of:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH), hepatic steatosis, fibrosis or cirrhosis);
(vi) lung conditions (Acute Lung Injury (ALI), Ventilator Induced Lung Injury (VILI), Radiation-Induced Lung Injury (RILI), pulmonary fibrosis (e.g. idiopathic pulmonary fibrosis));
(vii) kidney disease (e.g. chronic kidney disease).

In another aspect, the present invention provides a compound as defined herein, or a pharmaceutically acceptable salt or solvate thereof, for use in the treatment of:
(i) diabetes;
(ii) pulmonary arterial hypertension, acute coronary syndrome (ACS), congestive heart failure (CHF) or left ventricular hypertrophy;
(iii) inflammatory bowel disease (IBD), Crohn's Disease or ulcerative colitis;
(iv) breast cancer, prostate cancer or chronic lymphocytic leukaemia (CLL)
(v) non-alcoholic fatty liver disease (NAFLD), in particular Non-Alcoholic SteatoHepatitis (NASH), hepatic steatosis, fibrosis or cirrhosis);
(vi) psoriasis;
(vii) Acute Lung Injury (ALI), Ventilator Induced Lung Injury (VILI), Radiation-Induced Lung Injury (RILI), pulmonary fibrosis (e.g. idiopathic pulmonary fibrosis);
(viii) osteoarthritis or rheumatoid arthritis;
(ix) chronic kidney disease; or
(x) sepsis.

In another aspect, the present invention provides a compound as defined herein, or a pharmaceutically acceptable salt or solvate thereof, for use in the treatment of:
(i) diabetes;
(ii) pulmonary arterial hypertension;
(iii) inflammatory bowel disease (IBD), Crohn's Disease or ulcerative colitis;
(iv) non-alcoholic fatty liver disease (NAFLD), in particular Non-Alcoholic SteatoHepatitis (NASH);
(v) Acute Lung Injury (ALI), Ventilator Induced Lung Injury (VILI), Radiation-Induced Lung Injury (RILI); or
(vi) chronic kidney disease.

In another aspect, the present invention provides a compound as defined herein, or a pharmaceutically acceptable salt or solvate thereof, for use in the treatment of inflammatory bowel disease (IBD), Crohn's Disease or ulcerative colitis.

In another aspect, the present invention provides a compound as defined herein, or a pharmaceutically acceptable salt or solvate thereof, for use in the treatment of diabetes.

In another aspect, the present invention provides a compound as defined herein, or a pharmaceutically acceptable salt or solvate thereof, for use in the treatment of pulmonary arterial hypertension.

In another aspect, the present invention provides a compound as defined herein, or a pharmaceutically acceptable salt or solvate thereof, for use in the treatment of non-alcoholic fatty liver disease (NAFLD), in particular Non-Alcoholic SteatoHepatitis (NASH).

In another aspect, the present invention provides a compound as defined herein, or a pharmaceutically acceptable salt or solvate thereof, for use in the treatment of Acute Lung Injury (ALI), Ventilator Induced Lung Injury (VILI), or Radiation-Induced Lung Injury (RILI).

In another aspect, the present invention provides a compound as defined herein, or a pharmaceutically acceptable salt or solvate thereof, for use in the treatment of chronic kidney disease.

Suitably, the compound as defined herein, or a pharmaceutically acceptable salt or solvate thereof, is for use in the treatment of pulmonary arterial hypertension, IBD, crohn's disease, ulcerative colitis, diabetes (in particular in subjects with cardiovascular disease comorbidities), chronic kidney disease, ventilator induced lung injury (VILI) (e.g. in subjects who have been treated for COVID-19), acute lung injury (ALI), acute respiratory distress syndrome (ARDS), idiopathic pulmonary fibrosis, non-alcoholic fatty liver disease (including but not limited to hepatic steatosis, through inflammatory non-alcoholic steatohepatitis (NASH)), to fibrosis or cirrhosis) or radiation induced lung injury (RILI).

In another aspect, the present invention provides a compound as defined herein, or a pharmaceutically acceptable salt or solvate thereof, or a pharmaceutical composition as defined herein, for use in the suppression of eNAMPT activity. Suitably, the present invention provides a compound as defined herein, or a pharmaceutically acceptable salt or solvate thereof, or a pharmaceutical composition as defined herein, for use in the suppression of monomeric eNAMPT activity.

In another aspect, the present invention provides a method of suppressing eNAMPT activity in vitro or in vivo, said method comprising contacting a a sample comprising eNAMPT with an effective amount of a compound as defined herein, or a pharmaceutically acceptable salt or solvate thereof. Suitably, the method is a method of suppressing monomeric eNAMPT activity.

In a further aspect, the present invention provides a method of treating:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH));
(vi) inflammatory skin conditions (e.g. psoriasis);
(vii) lung conditions (Acute Lung Injury (ALI), Ventilator Induced Lung Injury (VILI), Radiation-Induced Lung Injury (RILI), pulmonary fibrosis (e.g. idiopathic pulmonary fibrosis));
(viii) arthritis (e.g. osteoarthritis or rheumatoid arthritis);
(ix) kidney disease (e.g. chronic kidney disease); or
(x) sepsis;
in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH), hepatic steatosis, fibrosis or cirrhosis);
(vi) lung conditions (Acute Lung Injury (ALI), Ventilator Induced Lung Injury (VILI), Radiation-Induced Lung Injury (RILI), pulmonary fibrosis (e.g. idiopathic pulmonary fibrosis)); or
(vii) kidney disease (e.g. chronic kidney disease);
in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating:
(i) diabetes;
(ii) pulmonary arterial hypertension, acute coronary syndrome (ACS), congestive heart failure (CHF) or left ventricular hypertrophy;
(iii) inflammatory bowel disease (IBD), Crohn's Disease or ulcerative colitis;
(iv) breast cancer, prostate cancer or chronic lymphocytic leukaemia (CLL)
(v) non-alcoholic fatty liver disease (NAFLD), in particular Non-Alcoholic SteatoHepatitis (NASH), hepatic steatosis, fibrosis or cirrhosis);
(vi) psoriasis;
(vii) Acute Lung Injury (ALI), Ventilator Induced Lung Injury (VILI), Radiation-Induced Lung Injury (RILI), pulmonary fibrosis (e.g. idiopathic pulmonary fibrosis);
(viii) osteoarthritis or rheumatoid arthritis;
(ix) chronic kidney disease; or
(x) sepsis;
   in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating:
(i) diabetes;
(ii) pulmonary arterial hypertension;
(iii) inflammatory bowel disease (IBD), Crohn's Disease or ulcerative colitis;
(iv) non-alcoholic fatty liver disease (NAFLD), in particular Non-Alcoholic SteatoHepatitis (NASH);
(v) Acute Lung Injury (ALI), Ventilator Induced Lung Injury (VILI), Radiation-Induced Lung Injury (RILI); or
(vi) chronic kidney disease;
   in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating inflammatory bowel disease (IBD), Crohn's Disease or ulcerative colitis in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating diabetes in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating pulmonary arterial hypertension in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating non-alcoholic fatty liver disease (NAFLD), in particular Non-Alcoholic SteatoHepatitis (NASH) in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, In a particular aspect, the present invention provides a method of treating chronic kidney disease in a patient in need of such treatment, said method comprising administering to said patient a therapeutically effective amount of a compound as defined herein, or a pharmaceutically acceptable salt or solvate thereof, or a pharmaceutical composition as defined herein.

In another aspect, the present invention provides the use of a compound as defined herein, or a pharmaceutically acceptable salt thereof, in the manufacture of a medicament for use in the treatment of:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH));
(vi) inflammatory skin conditions (e.g. psoriasis);
(vii) lung conditions (Acute Lung Injury (ALI), Ventilator Induced Lung Injury (VILI), Radiation-Induced Lung Injury (RILI), pulmonary fibrosis (e.g. idiopathic pulmonary fibrosis));
(viii) arthritis (e.g. osteoarthritis or rheumatoid arthritis);
(ix) kidney disease (e.g. chronic kidney disease); or
(x) sepsis.

In another aspect, the present invention provides the use of a compound as defined herein, or a pharmaceutically acceptable salt thereof, in the manufacture of a medicament for use in the treatment of:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH), hepatic steatosis, fibrosis or cirrhosis);
(vi) lung conditions (Acute Lung Injury (ALI), Ventilator Induced Lung Injury (VILI), Radiation-Induced Lung Injury (RILI), pulmonary fibrosis (e.g. idiopathic pulmonary fibrosis));
(vii) kidney disease (e.g. chronic kidney disease).

In another aspect, the present invention provides the use of a compound as defined herein, or a pharmaceutically acceptable salt thereof, in the manufacture of a medicament for use in the treatment of:
(i) diabetes;
(ii) pulmonary arterial hypertension, acute coronary syndrome (ACS), congestive heart failure (CHF) or left ventricular hypertrophy;
(iii) inflammatory bowel disease (IBD), Crohn's Disease or ulcerative colitis;
(iv) breast cancer, prostate cancer or chronic lymphocytic leukaemia (CLL)
(v) non-alcoholic fatty liver disease (NAFLD), in particular Non-Alcoholic SteatoHepatitis (NASH), hepatic steatosis, fibrosis or cirrhosis);
(vi) psoriasis;
(vii) Acute Lung Injury (ALI), Ventilator Induced Lung Injury (VILI), Radiation-Induced Lung Injury (RILI), pulmonary fibrosis (e.g. idiopathic pulmonary fibrosis);
(viii) osteoarthritis or rheumatoid arthritis;
(ix) chronic kidney disease; or
(x) sepsis.

In another aspect, the present invention provides the use of a compound as defined herein, or a pharmaceutically acceptable salt thereof, in the manufacture of a medicament for use in the treatment of:
(i) diabetes;
(ii) pulmonary arterial hypertension;
(iii) inflammatory bowel disease (IBD), Crohn's Disease or ulcerative colitis;
(iv) non-alcoholic fatty liver disease (NAFLD), in particular Non-Alcoholic SteatoHepatitis (NASH);
(v) Acute Lung Injury (ALI), Ventilator Induced Lung Injury (VILI), Radiation-Induced Lung Injury (RILI); or
(vi) chronic kidney disease.

In another aspect, the present invention provides the use of a compound as defined herein, or a pharmaceutically acceptable salt thereof, in the manufacture of a medicament for use in the treatment of inflammatory bowel disease (IBD), Crohn's Disease or ulcerative colitis.

In another aspect, the present invention provides the use of a compound as defined herein, or a pharmaceutically acceptable salt thereof, in the manufacture of a medicament for use in the treatment of diabetes.

In another aspect, the present invention provides the use of a compound as defined herein, or a pharmaceutically acceptable salt thereof, in the manufacture of a medicament for use in the treatment of pulmonary arterial hypertension.

In another aspect, the present invention provides the use of a compound as defined herein, or a pharmaceutically acceptable salt thereof, in the manufacture of a medicament for use in the treatment of non-alcoholic fatty liver disease (NAFLD), in particular Non-Alcoholic SteatoHepatitis (NASH).

In another aspect, the present invention provides the use of a compound as defined herein, or a pharmaceutically acceptable salt thereof, in the manufacture of a medicament for use in the treatment of Acute Lung Injury (ALI), Ventilator Induced Lung Injury (VILI), or Radiation-Induced Lung Injury (RILI).

In another aspect, the present invention provides the use of a compound as defined herein, or a pharmaceutically acceptable salt thereof, in the manufacture of a medicament for use in the treatment of chronic kidney disease.

In another aspect, the present invention provides the use of a compound as defined herein, or a pharmaceutically acceptable salt thereof, in the manufacture of a medicament for the suppression of eNAMPT activity.

### Routes of Administration

The compounds of the present invention, or pharmaceutical compositions comprising these compounds, may be administered to a subject by any convenient route of administration, whether systemically/peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### EXAMPLES

Reference is made to the accompanying figures, in which:

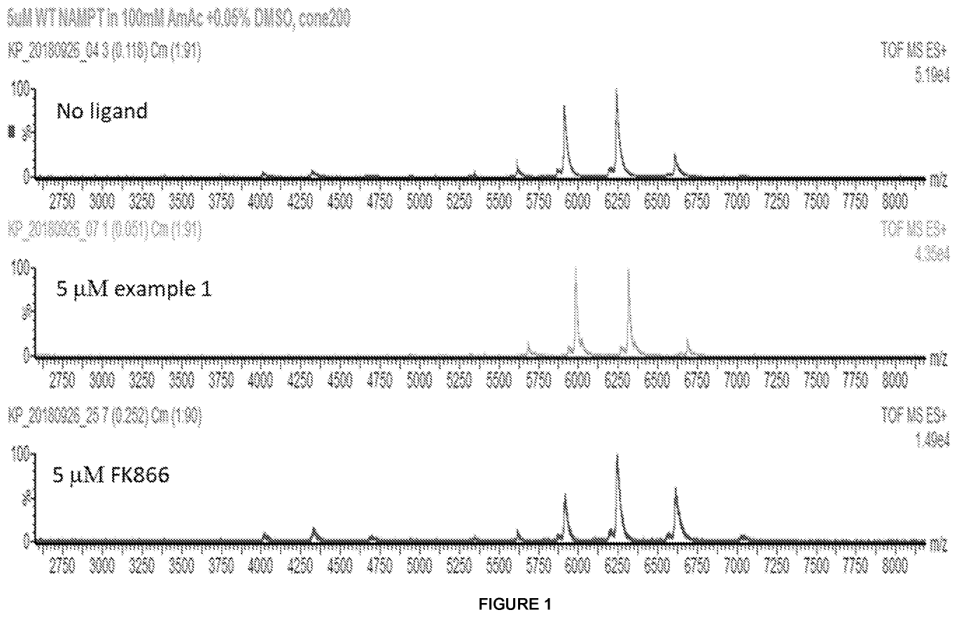

FIG. 1 shows native mass spectra of NAMPT alone (top), or incubated with example 1 (middle) or FK866 (bottom). The major peaks for NAMPT dimer are observed in the range 5,500 to 6,750 in all samples. The major peaks for NAMPT monomer are observed in the range 4,000 to 4,800, but are absent in the example 1 sample, indicating the absence of monomeric protein under these conditions.

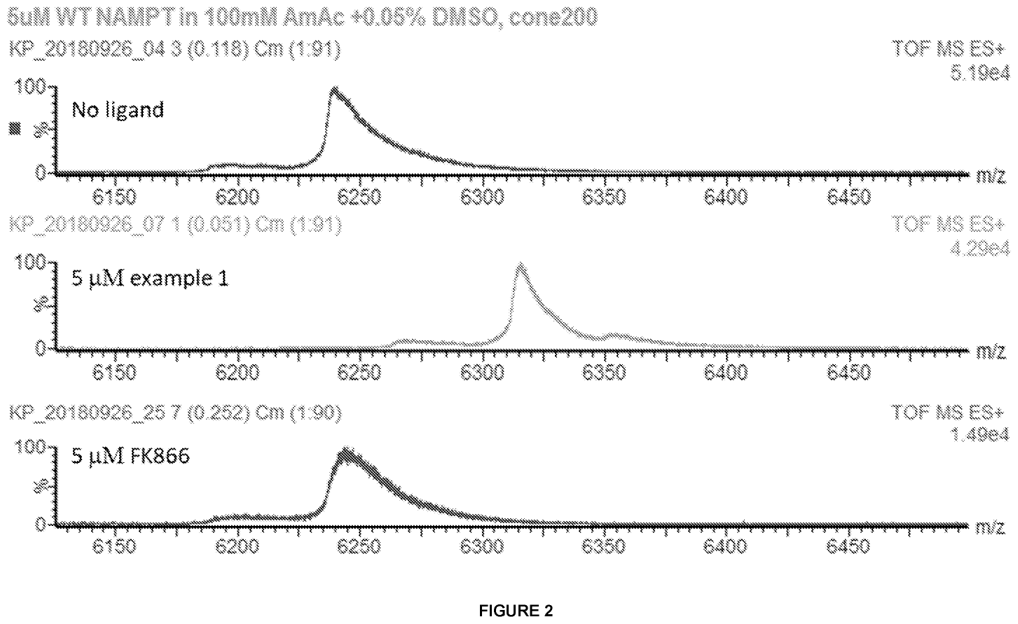

FIG. 2 shows the region from m/z 6125-6500 of the native mass spectra of NAMPT alone (top), or incubated with example 1 (middle) or FK866 (bottom). The major peak for NAMPT dimer is observed at ˜6240 (18+). Under these conditions the mass for the stable 2+2 complex between NAMPT and example 1 is observed at m/z ˜6320, whereas the FK866 complex dissociates resulting in detection of NAMPT only.

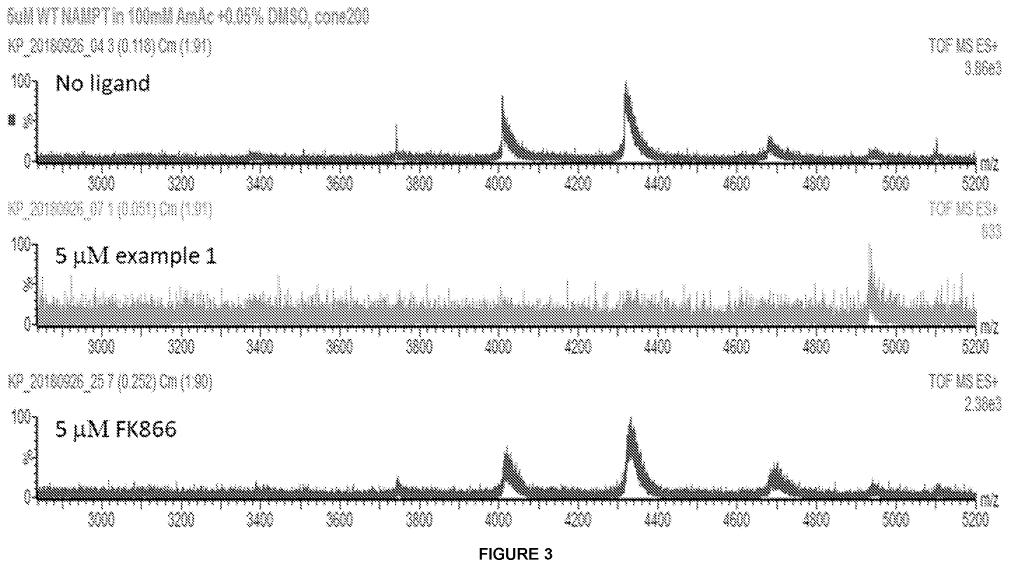

FIG. 3 shows the region from m/z 2950-5200 of the native mass spectra of NAMPT alone (top), or incubated with example 1 (middle) or FK866 (bottom). The major peaks for NAMPT monomer are observed in the range 4,000 to 4,800, but are absent in the example 1 sample, indicating the absence of monomeric protein under these conditions.

### SYNTHESIS OF STARTING MATERIALS

### Warheads:

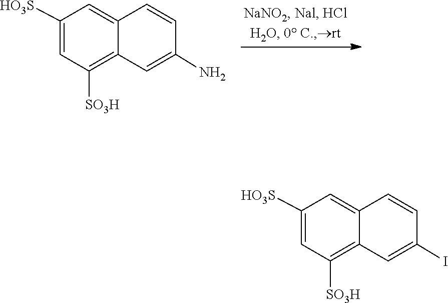 [SMILES: Nc1ccc2cc(S(=O)(=O)O)cc(S(=O)(=O)O)c2c1 ; O=S(=O)(O)c1cc(S(=O)(=O)O)c2cc(I)ccc2c1]

### 7-Iodonaphthalene-1,3-disulfonic acid (Intermediate A)

A suspension of 7-aminonaphthalene-1,3-disulfonic acid (16.5 mmol, 5.0 g) and HCl (37%, 3.6 mL) in H₂O (11 mL) was cooled down to 0° C. forming a white sludge. An ice-cooled solution of NaNO₂ (16.5 mmol, 1.1 g) in water (9 mL) was added dropwise via cannula. The mixture was stirred at 0° C. for 1 h. A solution of NaI (53.0 mmol, 7.9 g) and HCl (37%, 3.6 mL) in H₂O (15 mL) at 0° C. was added dropwise via cannula. The resulting dark mixture was allowed to reach RT, concentrated under reduced pressure and recrystallized on boiling water. The resulting pale-brown solid was washed with ice cooled water (40 mL), Et₂O (20 mL) and dried under air giving the product as a beige solid (3.1 g, 44% yield). ¹H NMR (400 MHz, DMSO-d₆) δ 9.18 (d, J=1.3 Hz, 1H), 8.23 (d, J=1.7 Hz, 1H), 8.09 (d, J=1.7 Hz, 1H), 7.78 (br. s, 2H).

### Tail Precursors:

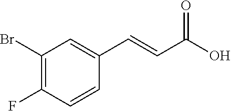 [SMILES: O=C(O)/C=C/c1ccc(F)c(Br)c1]

### (E)-3-(3-bromo-4-fluorophenyl)acrylic acid (Intermediate 1)

A mixture of 3-bromo-4-fluorobenzaldehyde (5.03 mmol, 930 mg), malonic acid (11.05 mmol, 1150 mg), piperidine (10 mol %, 0.05 mL) and pyridine (2.5 mL) was combined in a high pressure tube and stirred at 115° C. for 3 h. The mixture was allowed to reach RT and poured into 200 mL of 2M HCl. A white precipitate was formed, isolated by filtration and dried under vacuum giving the desired product (640 mg, 52%). ¹H NMR (500 MHz, DMSO-d₆) δ 12.49 (br., 1H), 8.13 (dd, J=6.8, 2.1 Hz, 1H), 7.79 (ddd, J=8.7, 4.9, 2.2 Hz, 1H), 7.57 (d, J=16.0 Hz, 1H), 7.43 (app. t, J=8.7 Hz, 1H), 6.60 (d, J=16.0 Hz, 1H); ¹³C NMR (101 MHz, DMSO-d₆) δ 167.8, 159.5 (d, J=248.9 Hz), 141.8, 133.7, 133.2 (d, J=3.8 Hz), 130.1 (d, J=7.8 Hz), 121.1, 117.6 (d, J=22.6 Hz), 109.2 (d, J=21.5 Hz); m/z (M+H)⁺ (ES⁺) 243.0, 245.0; t_{R}=2.35 min. HPLC Method 2 (Base).

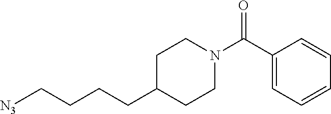 [SMILES: [N-]=[N+]=NCCCCC1CCN(C(=O)c2ccccc2)CC1]

### (4-(4-azidobutyl)piperidin-1-yl)(phenyl)methanone (Intermediate 2)

(4-(4-Hydroxybutyl)piperidin-1-yl)(phenyl)methanone (2.22 mmol, 545 mg), was dissolved in dry DMF (6 mL), degassed, and ice-cooled. DPPA (6.67 mmol, 1.4 mL), DBU (6.67 mmol, 1.0 mL) were added, the mixture stirred for 30 min at 0° C., followed by the addition of sodium azide (2.22 mmol, 144 mg) and stirring at 100° C. for 4 h. The mixture was allowed to reach RT, diluted with Et₂O (40 mL), washed with water (2×20 mL) and brine (30 mL), dried over MgSO₄, filtered, concentrated under reduced pressure and purified by reverse phase column chromatography (0.1% HCOOH modifier) affording the desired product as a brown oil (364 mg, 57%). ¹H NMR (400 MHz, CDCl₃) δ 7.45-7.37 (s, 5H), 4.80-4.67 (m, 1H), 3.82-3.70 (m, 1H), 3.30 (t, J=6.8 Hz, 2H), 3.03-2.90 (m, 1H), 2.82-2.69 (m, 1H), 1.90-1.78 (m, 1H), 1.76-1.49 (m, 4H), 1.47-1.38 (m, 2H), 1.36-1.01 (m, 4H); m/z (M+H)⁺ (ES⁺) 287.4; t_{R}=2.67 min. HPLC Method 2 (Base).

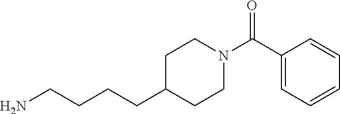 [SMILES: NCCCCC1CCN(C(=O)c2ccccc2)CC1]

### (4-(4-aminobutyl)piperidin-1-yl)(phenyl)methanone (Intermediate 3)

(4-(4-Azidobutyl)piperidin-1-yl)(phenyl)methanone (Intermediate 2; 1.27 mmol, 364 mg) was dissolved in MeOH:DCM (1:1, 10 mL) under an inert atmosphere. Pd/C (10% w/w, 10 mol %, 80 mg) was added and H₂ (1 atm) was bubbled through the solution. The mixture was stirred for 1 h, then filtered through Celite®, washed with DCM (20 mL), MeOH (20 mL) and concentrated under reduced pressure giving a colourless oil (184 mg, 55%). The crude was taken into the next step without further purification. m/z (M+H)⁺ (ES⁺) 261.4, 263.4; t_{R}=1.76 min. HPLC Method 2 (Base).

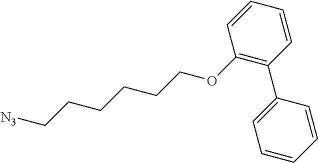 [SMILES: [N-]=[N+]=NCCCCCCOc1ccccc1-c1ccccc1]

### 2-((6-azidohexyl)oxy)-1,1′-biphenyl (Intermediate 4)

6-([1,1′-biphenyl]-2-yloxy)hexan-1-01 (0.89 mmol, 240 mg), was dissolved in dry DMF (3 mL), degassed, and ice-cooled. DPPA (2.68 mmol, 0.57 mL), DBU (2.68 mmol, 0.40 mL) were added, the mixture stirred for 30 min at 0° C., followed by the addition of sodium azide (0.89 mmol, 58 mg) and stirring at 100° C. for 4 h. The mixture was allowed to reach RT, diluted with Et₂O (40 mL), washed with water (2×20 mL) and brine (30 mL), dried over MgSO₄, filtered, concentrated under reduced pressure and used in the next step without further purification (200 mg, 76%). m/z (M+H)⁺ (ES⁺) 296.1; t_{R}=3.16 min. HPLC Method 2 (Base).

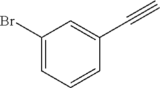

### 1-bromo-3-ethynylbenzene (Intermediate 5)

1-Bromo-3-iodobenzene (2.12 mmol, 0.27 mL), TEA (8.48 mmol, 1.19 mL), and ethynyltrimethylsilane (2.40 mmol, 0.32 mL) were dissolved in anhydrous THF (12 mL) and degassed with Ar for 20 min. PdCl₂(PPh₃)₂ (0.11 mmol, 74 mg) and CuI (0.06 mmol, 12 mg) were added and the mixture stirred at RT for 4 h. The mixture was concentrated, redissolved in Et₂O (30 mL) and washed with water (2×20 mL) and brine (20 mL). The organic phase was dried over MgSO₄, filtered, concentrated under reduced pressure and purified by filtering through a small pad of silica (ca. 2-3 g) and eluting with Hexane:AcOEt (95:5). The solution was concentrated under reduced pressure and redissolved in MeOH (10 mL). K2CO3 (5.30 mmol, 736 mg) was added and the mixture stirred for 1 h. The mixture was concentrated, filtered through a pad of silica (ca. 2-3 g) and eluted with Hex:AcOEt (95:5) affording a colourless oil (365 mg, 95%). m/z (M+H)⁺ (ES⁺) 181.3; t_{R}=3.25 min. HPLC Method 2 (Base).

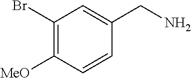 [SMILES: COc1ccc(CN)cc1Br]

### (3-bromo-4-methoxyphenyl)methanamine hydroformate (Intermediate 6)

1-Bromo-4-methoxybenzonitrile (1.0 g, 4.72 mmol) was dissolved in THF (20 mL). Borane (1M solution in THF, 14.15 mL) was added dropwise for 10 min at RT then the mixture was taken to reflux and stirred for 2 h. The mixture was cooled to 0° C. and slowly quenched with MeOH (10 mL) and concentrated under reduced pressure. The mixture was diluted with water (20 mL), extracted with CH₃Cl (3×15 mL) and washed with brine (15 mL). The organic phase was dried over MgSO₄, filtered, concentrated and purified by reverse phase chromatography (0.1% HCOOH modifier) affording the desired product as a white solid (443 mg, 43%). ¹H NMR (500 MHz, CDCl₃) δ 8.54 (s, 1H), 7.70 (d, J=2.2 Hz, 1H), 7.43 (dd, J=8.5, 2.3 Hz, 1H), 7.11 (d, J=8.5 Hz, 1H), 4.06 (s, 2H), 3.92 (s, 3H). m/z (M+H)⁺ (ES⁺) 216.2, 218.2; t_{R}=2.08 min. HPLC Method 2 (Base).

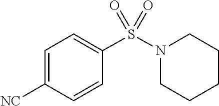 [SMILES: N#Cc1ccc(S(=O)(=O)N2CCCCC2)cc1]

### 4-(Piperidin-1-ylsulfonyl)benzonitrile (Intermediate 7)

3-Cyanobenzenesulfonyl chloride (500 mg, 2.47 mmol) was dissolved in DCM (6 mL). Piperidine (0.37 mL, 3.71 mmol) and TEA (0.69 mL, 4.94 mmol) were added to the mixture and stirred overnight. The mixture was concentrated under reduced pressure and purified by reverse phase chromatography (0.1% HCOOH modifier) affording the desired product as a white solid (470 mg, 76%). m/z: Compound does not ionise; t_{R}=2.39 min. HPLC Method 2 (Base).

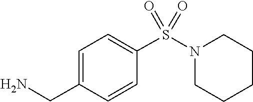 [SMILES: NCc1ccc(S(=O)(=O)N2CCCCC2)cc1]

### (4-(Piperidin-1-ylsulfonyl)phenyl)methanamine (Intermediate 8)

4-(Piperidin-1-ylsulfonyl)benzonitrile (Intermediate 7; 100 g, 0.40 mmol) was dissolved in THF (20 mL). Borane (1M solution in THF, 1.2 mL) was added dropwise for 10 min at RT then the mixture was taken to reflux and stirred for 1 h. The mixture was cooled to 0° C. and slowly quenched with MeOH (10 mL) and concentrated under reduced pressure. The mixture was diluted in MeOH (1 mL) and loaded onto a SCX cartridge (washed with 2 volumes of MeOH), washed with MeOH (3 mL), eluted with NH₃ in MeOH (7M, 2 mL) and concentrated under reduced pressure affording the desired product as a white solid (95 mg, 93%). m/z (M+H)⁺ (ES⁺) 255.3; t_{R}=1.68 min. HPLC Method 2 (Base).

### Tails:

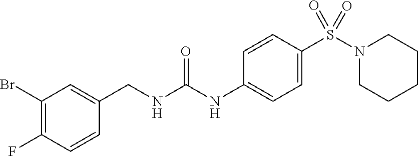 [SMILES: O=C(NCc1ccc(F)c(Br)c1)Nc1ccc(S(=O)(=O)N2CCCCC2)cc1]

### 1-(3-bromo-4-fluorobenzyl)-3-(4-(piperidin-1-ylsulfonyl)phenyl)urea (Intermediate 9)

3-Bromo-4-fluorobenzylamime hydrochloride (2.30 mmol, 553 mg) and TEA (5.06 mmol, 0.35 mL) were added into a −40° C., light protected solution of 4-(chlorosulfonyl)phenyl isocyanate (2.30 mmol, 500 mg) in THF (20 mL). The mixture was stirred for 2 h and allowed to reach RT before addition of piperidine (2.30 mmol, 0.23 mL) and TEA (5.06 mmol, 0.35 mL). The mixture was stirred for another 2 h then concentrated under reduced pressure, redissolved in DCM (ca. 30 mL) and washed with water (2×20 mL) and brine (20 mL), dried over MgSO₄, filtered, concentrated and purified by flash chromatography in silica gel (1% MeOH in DCM) affording the desired product as a white solid (458 mg, 42%). ¹H NMR (400 MHz, DMSO-d₆) δ 9.19 (s, 1H), 7.68-7.53 (m, 5H), 7.40-7.31 (m, 2H), 6.91 (t, J=6.0 Hz, 1H), 4.30 (d, J=5.9 Hz, 2H), 2.83 (t, J=5.3 Hz, 4H), 1.62-1.46 (m, 4H), 1.42-1.17 (m, 2H); ¹³C NMR (101 MHz, DMSO-d₆) δ 157.6 (d, J=243.4 Hz), 155.2, 145.1, 139.0 (d, J=3.5 Hz), 132.4, 129.2, 129.0 (d, J=7.4 Hz), 127.3, 117.7, 117.0 (d, J=22.3 Hz), 108.1 (d, J=20.9 Hz), 47.1, 42.1, 25.1, 23.4; m/z (M+H)⁺ (ES⁺) 470.2, 472.2; t_{R}=2.62 min. HPLC Method 2 (Base).

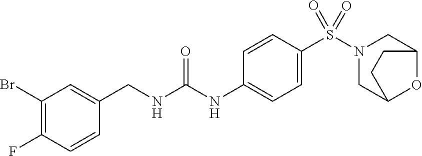 [SMILES: O=C(NCc1ccc(F)c(Br)c1)Nc1ccc(S(=O)(=O)N2CC3CCC(C2)O3)cc1]

### 1-(4-((8-oxa-3-azabicyclo[3.2.1]octan-3-yl)sulfonyl)phenyl)-3-(3-bromo-4-fluorobenzyl)urea (Intermediate 10)

3-Bromo-4-fluorobenzylamime hydrochloride (0.98 mmol, 200 mg) and DIPEA (1.03 mmol, 0.18 mL) were added into a −40° C., light protected solution of 4-(chlorosulfonyl)phenyl isocyanate (0.98 mmol, 213 mg) in MeCN (20 mL). The mixture was stirred for 2 h and allowed to reach RT before addition of 8-oxa-3-azabicyclo[3.2.1]octane (1.11 mmol, 122 mg) and DIPEA (1.03 mmol, 0.18 mL). The mixture was stirred for another 2 h then concentrated under reduced pressure, redissolved in DCM (ca. 30 mL) and washed with water (2×20 mL) and brine (20 mL), dried over MgSO₄, filtered, concentrated and purified by reverse phase column chromatography (0.1% HCOOH modifier). The product co-eluted with some impurities and was taken to the next step without further purification. m/z (M+H)⁺ (ES⁺) 498.1, 500.1; t_{R}=2.50 min. HPLC Method 2 (Base).

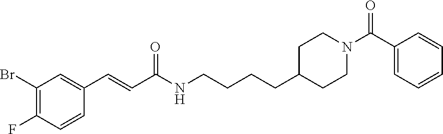 [SMILES: O=C(/C=C/c1ccc(F)c(Br)c1)NCCCCC1CCN(C(=O)c2ccccc2)CC1]

### (E)-N-(4-(1-benzoylpiperidin-4-yl)butyl)-3-(3-bromo-4-fluorophenyl)acrylamide (Intermediate 11)

A mixture of Intermediate 3 (0.77 mmol, 200 mg) and Intermediate 1 (0.77 mmol, 183 mg) were dissolved in anhydrous DMF (5 mL). HATU (1.15 mmol, 439 mg) and DIPEA (1.54 mmol, 0.26 mL) were added and the mixture stirred at RT overnight. The mixture was extracted with AcOEt (30 mL) and washed with brine (3×20 mL). The organic phase was dried over MgSO₄, filtered, concentrated under reduced pressure and purified by reverse phase column chromatography (0.1% HCOOH modifier) affording the desired compound as a colourless oil (300 mg, 80%). ¹H NMR (500 MHz, CDCl₃) δ 7.61 (dd, J=6.6, 2.1 Hz, 1H), 7.43 (d, J=15.6 Hz, 1H), 7.35-7.26 (m, 5H), 7.20 (s, 1H), 7.03 (app. t, J=8.3 Hz, 1H), 6.25 (d, J=15.5 Hz, 1H), 5.96 (t, J=5.9 Hz, 1H), 4.77-4.53 (m, 1H), 3.66 (d, J=13.4 Hz, 1H), 3.28 (app. q, J=6.7 Hz, 2H), 2.95-2.83 (m, 1H), 2.72-2.60 (m, 1H), 1.85-1.68 (m, 2H), 1.60-1.50 (m, 1H), 1.50-1.38 (m, 3H), 1.36-0.89 (m, 5H). m/z (M+H)⁺ (ES⁺) 487.3, 489.3; t_{R}=2.65 min. HPLC Method 2 (Base).

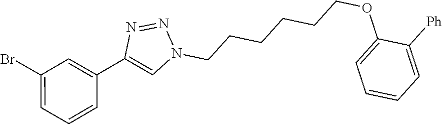

### 1-(6-([1,1′-biphenyl]-2-yloxy)hexyl)-4-(3-bromophenyl)-1H-1,2,3-triazole (Intermediate 12)

A mixture of Intermediate 4 (164 mg, 0.55 mmol), Intermediate 5 (100 mg, 0.55 mmol), copper sulfate (5 mg, 5 mol %), and sodium ascorbate (11 mg, 10 mol %) in THF:H₂O (5:1, 6 mL) was stirred at room temperature for 1 h. The reaction mixture was extracted with AcOEt (30 mL) and washed with brine (2×20 mL), dried over MgSO₄, filtered, concentrated under reduced pressure and purified by reverse phase column chromatography (0.1% formic acid modifier) affording the desired product as a colourless oil (96 mg, 37%). ¹H NMR (500 MHz, CDCl₃) δ 7.84 (app. t, J=1.8 Hz, 1H), 7.62 (app. dt, J=7.8, 1.3 Hz, 1H), 7.53 (s, 1H), 7.42-7.38 (m, 2H), 7.33 (app. dd, J=8.0, 2.1 Hz, 1H), 7.28-7.22 (m, 2H), 7.22-7.14 (m, 3H), 7.13 (s, 1H), 6.90 (app. td, J=7.5, 1.1 Hz, 1H), 6.83 (dd, J=8.2, 1.2 Hz, 1H), 4.18 (app. t, J=7.2 Hz, 2H), 3.83 (app. t, J=6.1 Hz, 2H), 1.76 (app. p, J=7.4 Hz, 2H), 1.62-1.54 (m, 2H), 1.36-1.26 (m, 2H), 1.24-1.15 (m, 2H); ¹³C NMR (126 MHz, CDCl₃) δ 155.9, 146.3, 138.7, 132.7, 131.0, 131.0, 130.9, 130.4, 129.6, 128.7, 128.6, 127.8, 126.8, 124.2, 122.9, 120.9, 119.8, 112.6, 68.1, 50.2, 30.1, 28.8, 25.9, 25.4; m/z (M+H)⁺ (ES⁺) 476.3; t_{R}=3.18 min. HPLC Method 2 (Base).

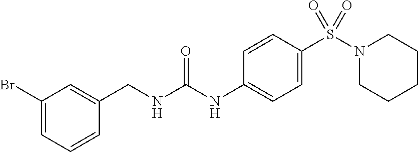 [SMILES: O=C(NCc1cccc(Br)c1)Nc1ccc(S(=O)(=O)N2CCCCC2)cc1]

### 1-(3-bromobenzyl)-3-(4-(piperidin-1-ylsulfonyl)phenyl)urea (Intermediate 13)

A mixture of 3-bromobenzylamine (201 mg, 1.08 mmol), phenyl (4-(cyclohexylsulfonyl)phenyl)carbamate (300 mg, 0.83 mmol), and TEA (0.35 mL, 2.49 mmol) in dioxane (10 mL) was stirred at 60° C. for 2 h. Upon cooling down the product crashed out as a white solid. The mixture was concentrated, redissolved in AcOEt (10 mL), washed with water (10 mL) and brine (10 mL). The organic phase was separated, dried over MgSO₄, filtered, concentrated under reduced pressure and purified by reverse phase column chromatography (0.1% ammonia modifier) affording the desired product as a white solid (190 mg, 50%). m/z (M+H)⁺ (ES⁺) 452.2, 454.4; t_{R}=2.63 min. HPLC Method 2 (Base).

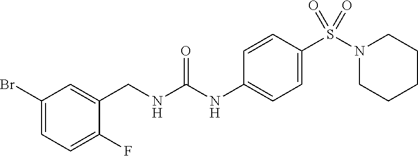 [SMILES: O=C(NCc1cc(Br)ccc1F)Nc1ccc(S(=O)(=O)N2CCCCC2)cc1]

### 1-(5-bromo-2-fluorobenzyl)-3-(4-(piperidin-1-ylsulfonyl)phenyl)urea (Intermediate 14)

A mixture of 5-bromo-2-fluorobenzylamine (216 mg, 0.90 mmol), phenyl (4-(cyclohexylsulfonyl)phenyl)carbamate (250 mg, 0.69 mmol), and TEA (0.29 mL, 2.07 mmol) in dioxane (10 mL) was stirred at 60° C. for 4 h. Upon cooling down the product crashed out as a white solid. The mixture was concentrated, redissolved in AcOEt (10 mL), washed with water (10 mL) and brine (10 mL). The organic phase was separated, dried over MgSO₄, filtered and concentrated under reduced pressure. The resulting white solid was triturated with Et₂O (ca. 10 mL) and dried giving the pure product (400 mg, 85%). m/z (M+H)⁺ (ES⁺) 470.2, 472.2; t_{R}=2.66 min. HPLC Method 2 (Base). ¹H NMR (500 MHz, CDCl₃) δ 7.66-7.60 (m, 2H), 7.53 (dd, J=6.6, 2.5 Hz, 1H), 7.52-7.48 (m, 2H), 7.41-7.31 (m, 1H), 7.16 (br. s, 1H), 6.95 (app. t, J=9.1 Hz, 1H), 4.49 (d, J=6.0 Hz, 2H), 2.96 (app. t, J=5.4 Hz, 4H), 1.69-1.63 (m, 4H), 1.43 (app. tq, J=8.8, 5.4, 4.4 Hz, 2H). m/z (M+H)⁺ (ES⁺) 470.2, 472.2; t_{R}=2.66 min. HPLC Method 2 (Base).

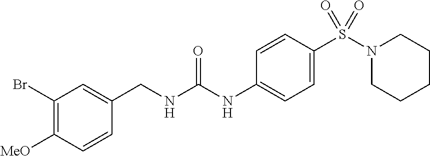 [SMILES: COc1ccc(CNC(=O)Nc2ccc(S(=O)(=O)N3CCCCC3)cc2)cc1Br]

### 1-(3-bromo-4-methoxybenzyl)-3-(4-(piperidin-1-ylsulfonyl)phenyl)urea (Intermediate 15)

A mixture of (3-bromo-4-methoxyphenyl)methanamine hydroformate (245 mg, 0.55 mmol), phenyl (4-(piperidin-1-ylsulfonyl)phenyl)carbamate (200 mg, 0.55 mmol), and TEA (0.23 mL, 1.65 mmol) in dioxane (10 mL) was stirred at 60° C. for 2 h. Upon cooling down the product crashed out as a white solid. The mixture was concentrated, redissolved in AcOEt (10 mL), washed with water (10 mL) and brine (10 mL). The organic phase was separated, dried over MgSO₄, filtered and concentrated under reduced pressure. The resulting white solid was purified by flash chromatography in silica gel (Hexane:AcOEt 2:3) affording the desired product as a colourless oil (262 mg, 99%). m/z (M+H)⁺ (ES⁺) 482.2, 484.2; t_{R}=2.59 min. HPLC Method 2 (Acid).

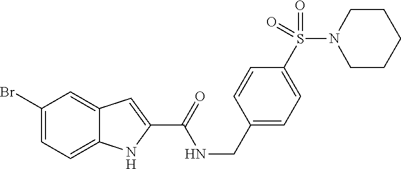 [SMILES: O=C(NCc1ccc(S(=O)(=O)N2CCCCC2)cc1)c1cc2cc(Br)ccc2[nH]1]

### 5-bromo-N-(4-(piperidin-1-ylsulfonyl)benzyl)-1H-indole-2-carboxamide (Intermediate 16)

A mixture of 5-bromo-1H-indole-2-carboxylic acid (60 mg, 0.37 mmol), intermediate 8 (95 mg, 0.37 mmol), HATU (200 mg, 0.56 mmol) and DIPEA (0.13 mL, 0.74 mmol) in DMF (3 mL) was stirred at RT overnight. The mixture extracted with Et₂O (20 mL), washed with water (10 mL) and brine (10 mL). The organic phase was separated, dried over MgSO₄, filtered and concentrated under reduced pressure. The resulting white solid was purified by reverse phase column chromatography (0.1% HCOOH modifier) affording the desired product as a pale yellow oil (57 mg, 32%). m/z (M+H)⁺ (ES⁺) 476.2, 478.2; t_{R}=2.70 min. HPLC Method 2 (Acid).

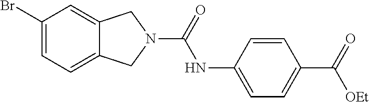 [SMILES: CCOC(=O)c1ccc(NC(=O)N2Cc3ccc(Br)cc3C2)cc1]

### Ethyl 4-(5-bromoisoindoline-2-carboxamido)benzoate (Intermediate 17)

5-bromoisoindoline (387 mg, 1.51 mmol), ethyl 4-isocyanatobenzoate (339 mg, 01.77 mmol) in THF (3 mL) were combined at 0° C. and the mixture was stirred at RT overnight. The mixture concentrated under reduced pressure, suspended on ice-cooled AcOEt (5 mL) and filtered. The resulting yellow solid was washed with ice-cooled AcOEt (5 mL) and taken to the next step without further purification (418 mg, 71%). m/z (M+H)⁺ (ES⁺) 389.2, 391.2; t_{R}=2.96 min. HPLC Method 2 (Base).

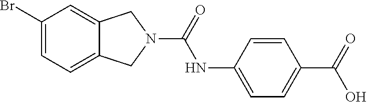 [SMILES: O=C(O)c1ccc(NC(=O)N2Cc3ccc(Br)cc3C2)cc1]

### 4-(5-bromoisoindoline-2-carboxamido)benzoic acid (Intermediate 18)

A solution of ethyl 4-(5-bromoisoindoline-2-carboxamido)benzoate (Intermediate 17; 200 mg, 0.51 mmol) in THF:MeOH:H₂O (3:1:1, 5 mL) was stirred with lithium hydroxide monohydrate (2.06 mmol, 84 mg) at RT overnight. The mixture was concentrated under reduced pressure, redissolved in H₂O (30 mL), washed with AcOEt (20 mL), acidified to pH 3, and extracted with AcOEt (2×30 mL). The organic phases were combined, dried on MgSO₄, filtered and concentrated under reduced pressure. The resulting yellow solid was taken to the next step without further purification (176 mg, 96%). m/z (M+H)⁺ (ES⁺) 375.2, 377.2; t_{R}=2.89 min. HPLC Method 2 (Base).

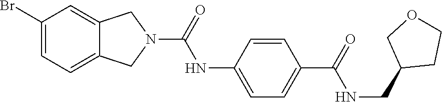 [SMILES: O=C(NC[C@@H]1CCOC1)c1ccc(NC(=O)N2Cc3ccc(Br)cc3C2)cc1]

### (S)-5-bromo-N-(4-(((tetrahydrofuran-3-yl)methyl)carbamoyl)phenyl)isoindoline-2-carboxamide (Intermediate 19)

4-(5-bromoisoindoline-2-carboxamido)benzoic acid (Intermediate 18; 120 mg, 0.33 mmol) was dissolved in anhydrous DMF (5 mL). HATU (0.50 mmol, 188 mg), DIPEA (1.32 mmol, 0.23 mL) and (S)-(tetrahydrofuran-3-yl)methanamine (0.40 mmol, 42 μL) were sequentially added and the mixture was stirred at RT for 3 h. The mixture was extracted with AcOEt (30 mL) and washed with brine (3×20 mL). The organic phase was dried over MgSO₄, filtered, concentrated under reduced pressure and purified by reverse phase column chromatography (0.1% HCOOH modifier) affording the desired compound as a pale yellow solid (120 mg, 82%). m/z (M+H)⁺ (ES⁺) 444.3, 446.3; t_{R}=2.72 min. HPLC Method 2 (Acid).

### Synthesis of NAMPT Inhibitors:

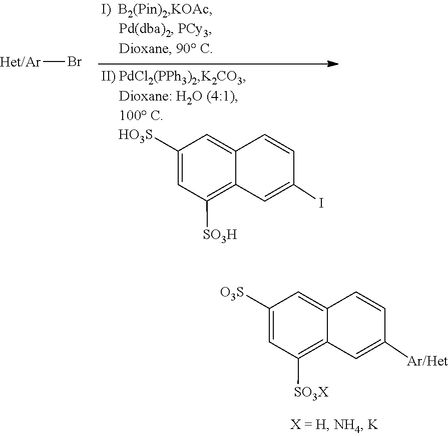

General Procedure: A solution of the corresponding haloarene/heteroarene derivative (1.0 eq), bis(pinacolato)diboron (1.5 eq) and potassium acetate (1.5-3.0 eq) in dioxane (0.1 M) was degassed with N₂ for 15 min. Bis(dibenzylidenacetone)palladium(0) (5 mol %) and triscyclohexylphosphine (10 mol %) were added and the mixture was stirred at 90° C. until all the starting haloderivative has been converted to the corresponding boronic ester (monitored by LCMS the formation of the boronic acid). The mixture was allowed to reach RT followed by the addition of 7-Iodonaphthalene-1,3-disulfonic acid (Intermediate A; 1.0-1.2 eq), bis(triphenylphosphine)palladium(II) dichloride (10 mol %), potassium carbonate (3.0 eq) and degassed water (0.3 M). The mixture was stirred at 100° C. until all the boronic este derivative has been converted to the desired product (monitored by LCMS). The mixture was allowed to reach RT, filtered, washed with MeOH (5 mL), concentrated under reduced pressure and purified by reverse phase column chromatography (5 to 100% MeCN in H₂O, modifier indicated) giving the corresponding disulfonate derivative.

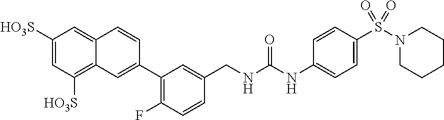 [SMILES: O=C(NCc1ccc(F)c(-c2ccc3cc(S(=O)(=O)O)cc(S(=O)(=O)O)c3c2)c1)Nc1ccc(S(=O)(=O)N2CCCCC2)cc1]

### 6-(2-fluoro-5-((3-(4-(piperidin-1-ylsulfonyl)phenyl)ureido)methyl)phenyl)-4-(trioxidaneylthio)naphthalene-2-sulfonic acid (Example 1)

Intermediate 9 (0.64 mmol, 300 mg), bis(pinacolato)diboron (0.96 mmol, 247 mg), potassium acetate (1.92 mmol, 188 mg), dioxane (4 mL), bis(dibenzylidenacetone)palladium(0) (0.06 mmol, 37 mg) and triscyclohexylphosphine (0.06 mmol, 13 mg) stirred for 2 h at 90° C. Then, 7-Iodonaphthalene-1,3-disulfonic acid (Intermediate A; 0.70 mmol, 291 mg), bis(triphenylphosphine)palladium(II) dichloride (0.06 mmol, 45 mg), potassium carbonate (1.92 mmol, 267 mg) and degassed water (1 mL) stirred for 1 h at 100° C. After purification by reverse phase column chromatography (0.1% NH₄OH modifier) the desired product was isolated as a white crystalline solid (264 mg, 61%). mp. 167-169° C.; ¹H NMR (400 MHz, DMSO-d₆) δ 9.17 (s, 1H), 8.97 (d, J=1.6 Hz, 1H), 8.29 (d, J=1.8 Hz, 1H), 8.15 (d, J=1.7 Hz, 1H), 8.05 (d, J=8.5 Hz, 1H), 7.68-7.60 (m, 3H), 7.59-7.54 (m, 2H), 7.48 (dd, J=7.6, 2.3 Hz, 1H), 7.43-7.37 (m, 1H), 7.32 (dd, J=10.4, 8.4 Hz, 1H), 6.99 (t, J=5.8 Hz, 1H), 4.39 (d, J=5.8 Hz, 2H), 2.82 (app. t, J=5.4 Hz, 4H), 1.52 (app. t, J=5.9 Hz, 4H), 1.39-1.29 (m, 2H); ¹³C NMR (101 MHz, DMSO-d₆) δ 158.7 (d, J=244.9 Hz), 155.2, 145.2, 144.8, 144.2, 137.1 (d, J=3.4 Hz), 133.5, 132.6, 130.3, 129.4-128.8 (m, 3C), 127.9, 127.2 (d, J=2.2 Hz), 127.1, 125.6, 123.8, 117.6, 117.6, 116.6, 116.4, 49.1, 47.0, 42.7, 25.1, 23.3; m/z (M+H)⁺ (ES⁺) 678.2; t_{R}=1.76 min. HPLC Method 1; HRMS (ES-TOF): m/z calcd. for C₂₉H₂₆FN₃O₉S: 337.5402, found 337.5408 [M−2H]²⁻. ¹H-NMR missing 2H signals from exchangeable protons.

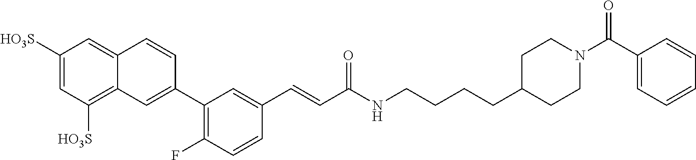 [SMILES: O=C(/C=C/c1ccc(F)c(-c2ccc3cc(S(=O)(=O)O)cc(S(=O)(=O)O)c3c2)c1)NCCCCC1CCN(C(=O)c2ccccc2)CC1]

### (E)-6-(5-(3-((4-(1-benzoylpiperidin-4-yl)butyl)amino)-3-oxoprop-1-en-1-yl)-2-fluorophenyl)-4-(trioxidaneylthio)naphthalene-2-sulfonic acid (Example 2)

Intermediate 11 (0.15 mmol, 70 mg), bis(pinacolato)diboron (0.21 mmol, 53 mg), potassium acetate (0.21 mmol, 21 mg), dioxane (3 mL), bis(dibenzylidenacetone)palladium(0) (0.02 mmol, 9 mg) and triscyclohexylphosphine (0.03 mmol, 6 mg) stirred for overnight at 90° C. Then, 7-Iodonaphthalene-1,3-disulfonic acid (Intermediate A; 0.15 mmol, 62 mg), bis(triphenylphosphine)palladium(II) dichloride (0.02 mmol, 10 mg), potassium carbonate (0.30 mmol, 42 mg) and degassed water (0.75 mL) stirred for 2 h at 100° C. After purification by reverse phase column chromatography (0.1% NH₄OH modifier) the desired product was isolated as a white crystalline solid (4.6 mg, 5%) as the diammonium salt. ¹H NMR (400 MHz, DMSO-d₆) δ 8.99 (s, 1H), 8.28 (d, J=1.7 Hz, 1H), 8.18-8.10 (m, 2H), 8.07 (d, J=8.5 Hz, 1H), 7.75-7.61 (m, 3H), 7.48 (d, J=15.8 Hz, 1H), 7.45-7.40 (m, 4H), 7.38-7.30 (m, 2H), 7.10 (s, 8H), 6.66 (d, J=15.8 Hz, 1H), 4.55-4.38 (m, 1H), 4.14-4.05 (m, 1H), 3.59-3.38 (m, 1H), 3.06-2.91 (m, 1H), 2.82-2.65 (m, 1H), 1.80-1.65 (m, 1H), 1.65-1.40 (m, 5H), 1.38-1.17 (m, 6H); m/z (M+H)⁺ (ES⁺) 695.3; t_{R}=2.21 min. HPLC Method 2 (Base).

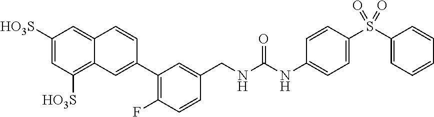 [SMILES: O=C(NCc1ccc(F)c(-c2ccc3cc(S(=O)(=O)O)cc(S(=O)(=O)O)c3c2)c1)Nc1ccc(S(=O)(=O)c2ccccc2)cc1]

### 6-(2-fluoro-5-((3-(4-(phenylsulfonyl)phenyl)ureido)methyl)phenyl)-4-(trioxidaneylthio) naphthalene-2-sulfonic acid (Example 3)

1-(3-bromo-4-fluorobenzyl)-3-(4-(phenylsulfonyl)phenyl)urea (0.13 mmol, 60 mg, prepared from phenyl (4-(phenylsulfonyl)phenyl)carbamate, CAS 1439358-24-3, using an analogous method to the synthesis of intermediate 9), bis(pinacolato)diboron (0.19 mmol, 50 mg), potassium acetate (0.26 mmol, 26 mg), dioxane (3 mL), bis(dibenzylidenacetone)palladium(0) (0.01 mmol, 7 mg) and triscyclohexylphosphine (0.03 mmol, 6 mg) stirred for 1 h at 90° C. Then, 7-Iodonaphthalene-1,3-disulfonic acid (Intermediate A; 0.13 mmol, 54 mg), bis(triphenylphosphine)palladium(II) dichloride (0.01 mmol, 9 mg), potassium carbonate (0.26 mmol, 36 mg) and degassed water (0.75 mL) stirred for 2 h at 100° C. After purification by reverse phase column chromatography (0.1% NH₄OH modifier) the desired product was isolated as a white crystalline solid (25 mg, 29%). ¹H NMR (500 MHz, DMSO-d₆) δ 9.21 (s, 1H), 8.97 (s, 1H), 8.30 (d, J=1.6 Hz, 1H), 8.16 (d, J=1.7 Hz, 1H), 8.05 (d, J=8.5 Hz, 1H), 7.90 (d, J=7.3 Hz, 2H), 7.79 (d, J=8.8 Hz, 2H), 7.69-7.55 (m, 6H), 7.47 (dd, J=7.6, 2.3 Hz, 1H), 7.42-7.35 (m, 1H), 7.31 (dd, J=10.4, 8.4 Hz, 1H), 7.00 (t, J=5.9 Hz, 1H), 4.37 (d, J=5.8 Hz, 2H); ¹³C NMR (126 MHz, DMSO-d₆) δ 158.7 (d, J=245.1 Hz), 155.1, 145.9, 144.7, 144.2, 142.6, 137.0 (d, J=3.2 Hz), 133.7, 133.5, 132.6 (d, J=2.2 Hz), 130.3 (d, J=2.5 Hz), 130.1, 129.2, 129.2, 129.1, 129.0, 129.0, 128.9, 127.9, 127.4, 127.3 (d, J=2.2 Hz), 125.6, 123.8, 118.0, 116.0 (d, J=22.7 Hz), 42.7; ¹⁹F NMR (471 MHz, DMSO-d₆) δ −120.53; m/z (M+H)⁺ (ES⁺) 688.2; t_{R}=1.77 min. HPLC Method 2 (Base).

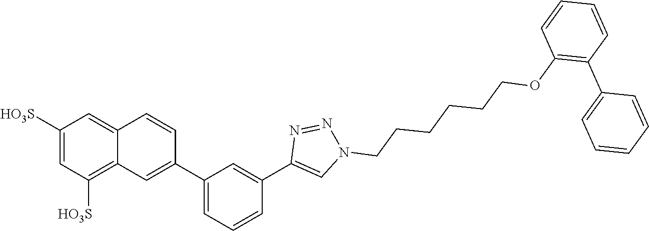 [SMILES: O=S(=O)(O)c1cc(S(=O)(=O)O)c2cc(-c3cccc(-c4cn(CCCCCCOc5ccccc5-c5ccccc5)nn4)c3)ccc2c1]

### 6-(3-(1-(6-([1,1′-biphenyl]-2-yloxy)hexyl)-1H-1,2,3-triazol-4-yl)phenyl)-4-(trioxidaneylthio) naphthalene-2-sulfonic acid (Example 4)

Intermediate 12 (0.19 mmol, 90 mg), bis(pinacolato)diboron (0.28 mmol, 72 mg), potassium acetate (0.38 mmol, 37 mg), dioxane (3 mL), bis(dibenzylidenacetone)palladium (0) (0.01 mmol, 7 mg) and triscyclohexylphosphine (0.02 mmol, 4 mg) stirred for 1 h at 90° C. Then, 7-Iodonaphthalene-1,3-disulfonic acid (Intermediate A; 0.19 mmol, 78 mg), bis(triphenylphosphine)palladium(II) dichloride (0.02 mmol, 13 mg), potassium carbonate (0.57 mmol, 78 mg) and degassed water (0.75 mL) stirred for 1 h at 100° C. After purification by reverse phase column chromatography (0.1% NH₄OH modifier) the desired product was isolated as a white crystalline solid (30 mg, 24%) as the diammonium salt. ¹H NMR (500 MHz, DMSO-d₆) δ 9.16 (s, 1H), 8.71 (s, 1H), 8.30 (s, 1H), 8.23 (s, 1H), 8.17 (s, 1H), 8.10 (d, J=8.5 Hz, 1H), 7.93-7.86 (m, 2H), 7.69 (app. dt, J=7.7, 1.5 Hz, 1H), 7.62 (app. t, J=7.7 Hz, 1H), 7.49 (d, J=6.8 Hz, 1H), 7.39 (app. t, J=7.6 Hz, 2H), 7.33-7.25 (m, 3H), 7.14 (br., 8H), 7.08 (d, J=8.1 Hz, 1H), 7.00 (app. td, J=7.4, 1.0 Hz, 1H), 4.39 (t, J=7.2 Hz, 2H), 3.96 (t, J=6.3 Hz, 2H), 1.92-1.81 (m, 2H), 1.69-1.60 (m, 2H), 1.42-1.36 (m, 2H), 1.34-1.24 (m, 2H); ¹³C NMR (126 MHz, DMSO-d₆) δ 155.9, 146.7, 144.5, 144.5, 144.3, 141.8, 138.7, 138.1, 132.7, 132.1, 130.9, 130.4, 130.1, 129.8, 129.7, 129.5, 129.3, 128.3, 127.2, 127.1, 125.7, 125.7, 124.8, 124.2, 123.8, 122.1, 121.2, 113.3, 68.1, 50.0, 30.0, 28.8, 25.9, 25.4; m/z (M+H)⁺ (ES⁺) 684.3; t_{R}=1.93 min. HPLC Method 2 (Base).

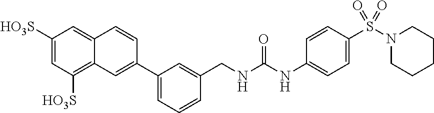 [SMILES: O=C(NCc1cccc(-c2ccc3cc(S(=O)(=O)O)cc(S(=O)(=O)O)c3c2)c1)Nc1ccc(S(=O)(=O)N2CCCCC2)cc1]

### 6-(3-((3-(4-(piperidin-1-ylsulfonyl)phenyl)ureido)methyl)phenyl)-4-(trioxidaneylthio) naphthalene-2-sulfonic acid (Example 5)

Intermediate 13 (0.22 mmol, 100 mg), bis(pinacolato)diboron (0.33 mmol, 84 mg), potassium acetate (0.44 mmol, 43 mg), dioxane (3 mL), bis(dibenzylidenacetone)palladium(0) (0.02 mmol, 13 mg) and triscyclohexylphosphine (0.04 mmol, 8 mg) stirred for 1 h at 100° C. Then, 7-Iodonaphthalene-1,3-disulfonic acid (Intermediate A; 0.22 mmol, 83 mg), bis(triphenylphosphine)palladium(II) dichloride (0.02 mmol, 13 mg), potassium carbonate (0.66 mmol, 83 mg) and degassed water (0.75 mL) stirred for 2 h at 100° C. After purification by reverse phase column chromatography (0.1% NH₄OH modifier) the desired product was isolated as a white crystalline solid (70 mg, 48%). ¹H NMR (500 MHz, DMSO-d₆) δ 9.16 (s, 1H), 9.13-9.04 (m, 1H), 8.27 (d, J=1.9 Hz, 1H), 8.14-8.10 (m, 1H), 8.04 (d, J=8.6 Hz, 1H), 7.82-7.77 (m, 1H), 7.69-7.59 (m, 4H), 7.56 (d, J=8.5 Hz, 2H), 7.50 (app. t, J=7.6 Hz, 1H), 7.36 (d, J=7.6 Hz, 1H), 6.97 (t, J=5.9 Hz, 1H), 4.43 (d, J=5.8 Hz, 2H), 2.82 (app. t, J=5.3 Hz, 4H), 1.52 (app. t, J=5.8 Hz, 4H), 1.33 (app. t, J=5.8 Hz, 2H); ¹³C NMR (126 MHz, DMSO-d₆) δ 155.3, 145.3, 144.6, 144.3, 141.2, 141.2, 138.2, 132.6, 129.7, 129.5, 129.5, 129.2, 129.2, 127.1, 126.9, 126.5, 126.2, 125.7, 125.5, 123.8, 117.6, 47.1, 43.4, 25.1, 23.3; m/z (M+H)⁺ (ES⁺) 660.2; t_{R}=1.77 min. HPLC Method 2 (Base).

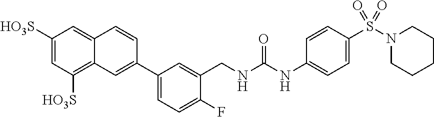 [SMILES: O=C(NCc1cc(-c2ccc3cc(S(=O)(=O)O)cc(S(=O)(=O)O)c3c2)ccc1F)Nc1ccc(S(=O)(=O)N2CCCCC2)cc1]

### 6-(4-fluoro-3-((3-(4-(piperidin-1-ylsulfonyl)phenyl)ureido)methyl)phenyl)-4-(trioxidaneylthio) naphthalene-2-sulfonic acid (Example 6)

Intermediate 14 (0.21 mmol, 100 mg), bis(pinacolato)diboron (0.32 mmol, 81 mg), potassium acetate (0.63 mmol, 62 mg), dioxane (3 mL), bis(dibenzylidenacetone)palladium(0) (0.02 mmol, 12 mg) and triscyclohexylphosphine (0.04 mmol, 8 mg) stirred for 1 h at 90° C. Then, 7-Iodonaphthalene-1,3-disulfonic acid (Intermediate A; 0.25 mmol, 110 mg), bis(triphenylphosphine)palladium(II) dichloride (0.02 mmol, 15 mg), potassium carbonate (0.63 mmol, 87 mg) and degassed water (0.75 mL) stirred for 2 h at 100° C. After purification by reverse phase column chromatography (0.1% NH₄OH modifier) the desired product was isolated as a white crystalline solid (96 mg, 67%). ¹H NMR (500 MHz, DMSO-d₆) δ 9.24 (s, 1H), 9.02 (d, J=1.8 Hz, 1H), 8.24 (d, J=1.8 Hz, 1H), 8.08 (d, J=1.8 Hz, 1H), 7.97 (d, J=8.5 Hz, 1H), 7.73-7.67 (m, 2H), 7.59 (app. d, J=8.7 Hz, 3H), 7.51 (d, J=8.5 Hz, 2H), 7.31 (t, J=9.2 Hz, 1H), 7.12-7.04 (m, 1H), 4.42 (d, J=5.1 Hz, 2H), 2.77 (app. t, J=5.4 Hz, 4H), 1.47 (app. t, J=5.7 Hz, 4H), 1.34-1.25 (m, 2H); ¹⁹F NMR (471 MHz, DMSO-d₆) δ −121.08; m/z (M+H)⁺ (ES⁺) 678.2; t_{R}=1.68 min. HPLC Method 2 (Base).

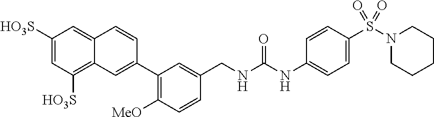 [SMILES: COc1ccc(CNC(=O)Nc2ccc(S(=O)(=O)N3CCCCC3)cc2)cc1-c1ccc2cc(S(=O)(=O)O)cc(S(=O)(=O)O)c2c1]

### 6-(2-methoxy-5-((3-(4-(piperidin-1-ylsulfonyl)phenyl)ureido)methyl)phenyl)-4-(trioxidaneylthio)naphthalene-2-sulfonic acid (Example 7)

Intermediate 15 (0.30 mmol, 145 mg), bis(pinacolato)diboron (0.45 mmol, 115 mg), potassium acetate (0.90 mmol, 88 mg), dioxane (3 mL), bis(dibenzylidenacetone)palladium(0) (0.03 mmol, 17 mg) and triscyclohexylphosphine (0.06 mmol, 12 mg) stirred for 3 h at 90° C. Then, 7-Iodonaphthalene-1,3-disulfonic acid (Intermediate A; 0.33 mmol, 136 mg), bis(triphenylphosphine)palladium(II) dichloride (0.03 mmol, 21 mg), potassium carbonate (0.90 mmol, 125 mg) and degassed water (0.75 mL) stirred for 2 h at 100° C. After purification by two reverse phase column chromatography (first 0.1% NH₄OH modifier, second 0.1 HCOOH) the desired product was isolated as a white crystalline solid (3 mg, 2%). ¹H NMR (500 MHz, DMSO-d₆) δ 9.07 (s, 1H), 8.83 (d, J=1.7 Hz, 1H), 8.24 (d, J=1.6 Hz, 1H), 8.09 (s, 1H), 7.93 (d, J=8.5 Hz, 1H), 7.64-7.58 (m, 2H), 7.58-7.53 (m, 3H), 7.34 (dd, J=8.4, 2.3 Hz, 1H), 7.27 (d, J=2.2 Hz, 1H), 7.12 (d, J=8.5 Hz, 1H), 6.86 (br., 1H), 4.33 (d, J=4.2 Hz, 2H), 3.76 (s, 3H), 2.82 (app. t, J=5.5 Hz, 4H), 1.57-1.45 (m, 4H), 1.40-1.28 (m, 2H); m/z (M+H)⁺ (ES⁺) 690.2; t_{R}=1.74 min. HPLC Method 2 (Base).

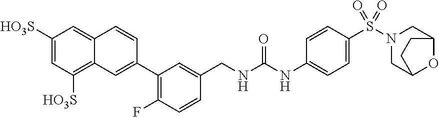 [SMILES: O=C(NCc1ccc(F)c(-c2ccc3cc(S(=O)(=O)O)cc(S(=O)(=O)O)c3c2)c1)Nc1ccc(S(=O)(=O)N2CC3CCC(C2)O3)cc1]

### 6-(5-((3-(4-((8-oxa-3-azabicyclo[3.2.1]octan-3-yl)sulfonyl)phenyl)ureido)methyl)-2-fluorophenyl)-4-(trioxidaneylthio)naphthalene-2-sulfonic acid (Example 8)

Intermediate 10 (0.30 mmol, 150 mg), bis(pinacolato)diboron (0.45 mmol, 115 mg), potassium acetate (0.90 mmol, 88 mg), dioxane (3 mL), bis(dibenzylidenacetone)palladium(0) (0.03 mmol, 17 mg) and triscyclohexylphosphine (0.06 mmol, 12 mg) stirred for 3 h at 90° C. Then, 7-Iodonaphthalene-1,3-disulfonic acid (Intermediate A; 0.30 mmol, 124 mg), bis(triphenylphosphine)palladium(II) dichloride (0.03 mmol, 21 mg), potassium carbonate (0.90 mmol, 125 mg) and degassed water (0.75 mL) stirred for 1 h at 100° C. After purification by two reverse phase column chromatography (0.1% NH₄OH modifier) the desired product was isolated as a white crystalline solid (12 mg, 6%) as the diammonium salt. ¹H NMR (400 MHz, DMSO-d₆) δ 9.19 (s, 1H), 8.98 (d, J=1.7 Hz, 1H), 8.30 (d, J=1.8 Hz, 1H), 8.16 (d, J=1.8 Hz, 1H), 8.06 (d, J=8.5 Hz, 1H), 7.68-7.62 (m, 3H), 7.58-7.52 (m, 2H), 7.50 (dd, J=7.6, 2.3 Hz, 1H), 7.45-7.37 (m, 1H), 7.34 (dd, J=10.4, 8.4 Hz, 1H), 7.17 (s, 8H), 6.99 (t, J=5.9 Hz, 1H), 4.41 (d, J=5.8 Hz, 2H), 4.33 (s, 1H), 3.27-3.14 (m, 2H), 2.43 (dd, J=11.4, 2.3 Hz, 2H), 1.86-1.71 (m, 4H); ¹⁹F NMR (376 MHz, DMSO) δ −120.51; m/z (M+H)⁺ (ES⁺) 706.2; t_{R}=1.68 min. HPLC Method 2 (Base).

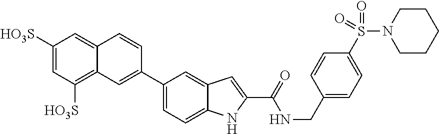 [SMILES: O=C(NCc1ccc(S(=O)(=O)N2CCCCC2)cc1)c1cc2cc(-c3ccc4cc(S(=O)(=O)O)cc(S(=O)(=O)O)c4c3)ccc2[nH]1]

### 6-(2-((4-(piperidin-1-ylsulfonyl)benzyl)carbamoyl)-1H-indol-5-yl)-4-(trioxidaneylthio) naphthalene-2-sulfonic acid (Example 9)

Intermediate 16 (0.12 mmol, 55 mg), bis(pinacolato)diboron (0.17 mmol, 44 mg), potassium acetate (0.36 mmol, 35 mg), dioxane (3 mL), bis(dibenzylidenacetone)palladium(0) (0.01 mmol, 7 mg) and triscyclohexylphosphine (0.01 mmol, 2 mg) stirred for 2 h at 90° C. Then, 7-Iodonaphthalene-1,3-disulfonic acid (Intermediate A; 0.13 mmol, 54 mg), bis(triphenylphosphine)palladium(II) dichloride (0.01 mmol, 8 mg), potassium carbonate (0.39 mmol, 54 mg) and degassed water (0.75 mL) stirred at 100° C. overnight. After purification by reverse phase column chromatography (0.1% NH₄OH modifier) the desired product was isolated as a pale yellow oil (8 mg, 11%) as the diammonium salt. ¹H NMR (400 MHz, DMSO-d₆) δ 11.80 (s, 1H), 9.28 (app. t, J=6.1 Hz, 1H), 9.13 (s, 1H), 8.27 (s, 1H), 8.14 (s, 1H), 8.05 (d, J=8.5 Hz, 1H), 8.01 (s, 1H), 7.92-7.89 (m, 1H), 7.85-7.79 (m, 1H), 7.74 (d, J=8.1 Hz, 2H), 7.65-7.60 (m, 3H), 7.34 (s, 1H), 4.67 (d, J=6.0 Hz, 2H), 2.89 (app. t, J=5.4 Hz, 4H), 1.60-1.49 (m, 4H), 1.43-1.34 (m, 2H); m/z (M+H)⁺ (ES⁺) 684.3; t_{R}=1.69 min. HPLC Method 2 (Base).

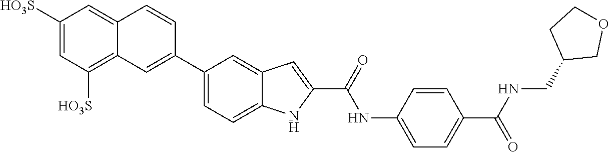 [SMILES: O=C(NC[C@@H]1CCOC1)c1ccc(NC(=O)c2cc3cc(-c4ccc5cc(S(=O)(=O)O)cc(S(=O)(=O)O)c5c4)ccc3[nH]2)cc1]

### (S)-6-(2-((4-(((Tetrahydrofuran-3-yl)methyl)carbamoyl)phenyl)carbamoyl)isoindolin-5-yl)-4-(trioxidaneylthio)naphthalene-2-sulfonic acid (Example 10)

Intermediate 19 (0.22 mmol, 96 mg), bis(pinacolato)diboron (0.32 mmol, 82 mg), potassium acetate (0.66 mmol, 65 mg), dioxane (3 mL), bis(dibenzylidenacetone)palladium(0) (0.02 mmol, 13 mg) and triscyclohexylphosphine (0.02 mmol, 4 mg) stirred for 1.5 h at 80° C. Then, 7-Iodonaphthalene-1,3-disulfonic acid (Intermediate A; 0.24 mmol, 100 mg), bis(triphenylphosphine)palladium(II) dichloride (0.02 mmol, 15 mg), potassium carbonate (0.66 mmol, 92 mg) and degassed water (0.75 mL) stirred at 100° C. for 1.5 h. After purification by reverse phase column chromatography (0.1% NH₄OH modifier) the desired product was isolated as a white solid (20 mg, 14%) as the diammonium salt. ¹H NMR (500 MHz, DMSO) δ 9.13 (s, 1H), 8.66 (s, 1H), 8.44 (app. t, J=5.8 Hz, 1H), 8.29 (d, J=1.7 Hz, 1H), 8.16 (s, 1H), 8.08 (d, J=8.6 Hz, 1H), 7.86 (dd, J=8.6, 1.9 Hz, 1H), 7.82-7.77 (m, 2H), 7.74-7.67 (m, 4H), 7.53 (d, J=7.9 Hz, 1H), 4.91 (s, 2H), 4.87 (s, 2H), 3.79-3.71 (m, 1H), 3.69 (dd, J=8.5, 6.9 Hz, 1H), 3.66-3.58 (m, 1H), 3.49 (dd, J=8.5, 5.2 Hz, 1H), 3.31-3.17 (m, 2H), 2.51-2.43 (m, 1H), 2.00-1.87 (m, 1H), 1.67-1.54 (m, 1H); ¹³C NMR (126 MHz, DMSO) δ 166.5, 154.1, 144.5, 144.2, 143.7, 140.4, 138.2, 138.0, 136.7, 132.6, 129.8, 129.5, 128.2, 127.9, 126.9, 125.6, 125.6, 125.5, 123.9, 123.8, 121.7, 118.7, 71.0, 67.3, 42.3, 40.9, 39.4, 30.0; m/z (M+H)⁺ (ES⁺) 652.3; t_{R}=1.54 min. HPLC Method 2 (Base).

### Native Mass Spectrometry

The protein was expressed and isolated as previously described and, on the day of analysis, the buffer was exchanged into 100 mM ammonium acetate (Fisher Scientific, Loughborough, UK) pH 6.9 using micro Bio-Spin Chromatography columns (Micro Bio-Spin 6 Columns, Bio-Rad, Watford, UK) following the instructions specified by the manufacturer. The procedure was repeated twice and diluted to give a final concentration of NAMPT (5 μmol/l), which was incubated with compound (5 μmol/l) for 12 hours prior to analysis. Native MS data was acquired on the Synapt G2S HDMS (Waters, Manchester, UK). NanoESI capillaries were prepared in-house from thin-walled borosilicate capillaries (inner diameter 0.9 mm, outer diameter 1.2 mm, World Precision Instruments, Stevenage, UK) using a Flaming/Brown P-1000 micropipette puller (Sutter Instrument Company, Novato, CA, USA). A positive voltage was applied to the solution via a platinum wire (Goodfellow Cambridge Ldt, Huntington, UK) inserted into the capillary. Gentle source conditions were applied to preserve the native-like structure: capillary voltage 1.2-1.5 kV, sampling cone 50-200 V, source temperature 70° C. Trap collision energy was 4 V, transfer collision energy was set to 0 V. Nitrogen was the carrier gas. External calibration of the spectra was achieved using solutions of cesium iodide (2 mg/mL in 50:50 water:isopropanol). Data were acquired and processed with MassLynx software (Waters, Manchester, UK). FIGS. 1, 2 and 3 depict the mass spectra for NAMPT alone or incubated with FK866 ((E)-N-[4-(1-benzoylpiperidin-4-yl)butyl]-3-pyridin-3-ylprop-2-enamide, which is a known NAMPT inhibitor used for comparative purposes) or 6-(2-fluoro-5-((3-(4-(piperidin-1-ylsulfonyl)phenyl)ureido)methyl)phenyl)-4-(trioxidaneylthio)naphthalene-2-sulfonic acid (Example 1).

### Enzyme Assay

In a 96-well opaque black plate, NAMPT (30 nM—all concentrations provided as final), PRPP (50 μM) and ATP (2 mM) with or without test compounds (11 concentrations, prepared by three-fold dilutions from final concentration of 30 mM, all in triplicate) were incubated for 20 minutes at 37° C. in TMD buffer (50 mM Tris-HCl, 10 mM MgCl₂, 2 mM DTT, pH 7.5). The enzymatic reaction was initiated by the addition of NAM (25 μM) and the plate incubated for 20 minutes at 37° C. 20 μL of 20% acetophenone (in DMSO) and 20 μl of 2 M KOH were added to each well and incubated to ambient temperature of 5 minutes, then 90 μl of 100% formic acid was added to each well and the plate incubated at 37° C. for 20 minutes before reading on a Hides Sense plate reader (Ex/Em=355/460 nm). Data was processed to % control in Excel and IC₅₀ curves fitted using GraphPad Prism. Data is reported from fitting of n=3 separate repeats.

### Tm Shift Assay

Thermal melting experiments were carried out using an Applied Biosystem StepOnePlus qPCR instrument. NAMPT (1 μM) was buffered in 10 mM HEPES, pH 7.5, 140 mM NaCl and assayed in a 96-well plate at a final concentration of 2 μM in a 50 μL volume.

Compounds were added at a final concentration of 100 μM and SYPRO Orange was added as a fluorescence probe at a dilution of 1:5000 (v/v). The temperature was raised with a step of 1° C. per minute from 25 to 96° C., and fluorescence readings were taken at each interval. Experiments were performed in triplicate, and the observed temperature shifts were recorded as the difference between the transition midpoints of sample and reference wells containing protein without ligand in the same plate and determined by nonlinear least-squares fit, reported in ° C. as the mean of the values obtained from 3 independent repeats.

### THP-1 WST-1 Assay

THP-1 cells were plated at 30,000 cells per well (400,000 cells per mL) in a final volume of 150 μL media (RPMI-1640 containing 10% v/v FBS) containing 1 μM test compound. Samples were incubated for 48 hrs at which point 15 μL WST-1 solution (Sigma-Aldrich) was added. Light was excluded and the samples incubated for a further 2 hrs. Absorbance of wells were read at 450 nm and 630 nm using a Hidex Sense plate reader. Metabolic inhibition was determined by the absorbance at 450 nM and transformed to % control using Excel. Data reported is an average of three replicates.

| | | NAMPT | THP-1 WST-1 |
|---|---|---|---|
| | NAMPT Tm shift | enzymatic | (% inhibition |
| Example | (° C.) | IC₅₀ | at 1 μM) |
| Comparative | 2.6 | 1.6*** | 79.6% |
| Example: | | | |
| FK866¹ | | | |
| 1 | 10.3 | 511 | <0% |
| 2 | 9.1 | 8358 | 70.4 |
| 3 | 12.2 | ND | <0% |
| 4 | ND* | ND | <0% |
| 5 | 10.8 | ND | <0% |
| 6 | 9.6 | ND | 1.0% |
| 7 | 7.5 | ND | <0% |
| 8 | 9.7 | ND | <0% |
| 9 | 4.2** | ND | <0% |
| 10 | 20.5** | ND | ND |

*Comparative fluorescence prevents detection in Sypro orange channel.
**Compounds effectively completely supress the melt event, Tm shift values fitted to resultant profile but with low confidence.
***Literature value; Acta Pharmacologica Sinica (2018) 39: 394-301.
¹FK866 is the compouond (E)-N-[4-(1-benzoylpiperidin-4-yl)butyl]-3-pyridin-3-ylprop-2-enamide, which is a known NAMPT inhibitor used for comparative purposes.

1. A compound of formula I, or a pharmaceutically acceptable salt or solvate thereof, as shown below: 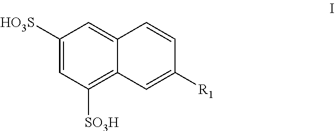
wherein R₁ is selected from:
(i) a group of the formula II: 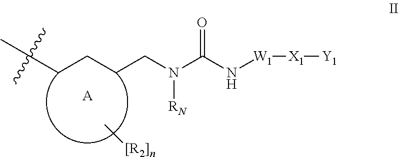
wherein:
Ring A is selected from phenyl or a 5- or 6-membered heteroaryl;
n is 0, 1 or 2;
each R₂ group, when present, is selected from: halo, nitro, cyano, R₂ₐ, —[CH₂]_{q}—NR₂ₐR_{2b}, —[CH₂]_{q}—OR₂ₐ, —[CH₂]_{q}—C(O)R₂ₐ, —[CH₂]_{q}—C(O)OR₂ₐ, —[CH₂]_{q}—OC(O)R₂ₐ, —[CH₂]_{q}—C(O)N(R_{2b})R₂ₐ, —[CH₂]_{q}—N(R_{2b})C(O)R₂ₐ, —[CH₂]_{q}—N(R_{2c})—C(O)—N(R_{2b})R₂ₐ, —[CH₂]_{q}—S(O)ₚR₂ₐ (where p is 0, 1 or 2), —[CH₂]_{q}—SO₂N(R₂)R₂ₐ, —[CH₂]_{q}—N(R_{2b})SO₂R₂ₐ;
wherein q is 0, 1, 2 or 3;
R₂ₐ is hydrogen or (1-4C)alkyl optionally substituted by halo, hydroxy, amino or cyano; and
R_{2b} and R_{2c} are hydrogen or (1-2C)alkyl;
R_{N} is selected from hydrogen or methyl;
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, (1-2C)alkyl, (1-2C)haloalkyl, cyano or amino;
W₁ is: 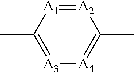
wherein A₁, A₂, A₃ or A₄ are selected from CH, N or C—F, with the provisos that:
only one or two of A₁, A₂, A₃ or A₄ can be N; and
only one or two of A₁, A₂, A₃ or A₄ can be C—F;
X₁ is a linker group of the formula:
—[CH₂]ₙ₁-L₁-[CH₂]ₙ₂—
wherein
n1 and n2 are selected from 0 or 1;
L₁ is selected from —O—, —S—, —SO—, —SO₂—, —N(Rₓ₁ₐ)—, —C(O)—, —C(O)O—, —OC(O)—, —C(O)N(Rₓ₁ₐ)—, —N(Rₓ₁ₐ)C(O)—, —N(R_{x1b})C(O)N(Rₓ₁ₐ)—, —N(Rₓ₁ₐ)C(O)O—, —OC(O)N(Rₓ₁ₐ)—, —S(O)₂N(Rₓ₁ₐ), —N(Rₓ₁ₐ)SO₂—, or —C(O)N(Rₓ₁ₐ)SO₂—, or —SO₂N(Rₓ₁ₐ)C(O)—; and wherein Rₓ₁ₐ and R_{x1b} are each independently selected from hydrogen or (1-2C)alkyl;
Y₁ is selected from a carbocyclic, aryl, heteroaryl or heterocyclic ring which is optionally substituted by halo, nitro, cyano, R_{y1a}, —[CH₂]ᵣ—NR_{y1a}R_{y1b}, —[CH₂]ᵣ—OR_{y1a}, —[CH₂]ᵣ—C(O)R_{y1a}, —[CH₂]ᵣ—C(O)OR_{y1a}, —[CH₂]ᵣ—OC(O)R_{y1a}, —[CH₂]ᵣ—C(O)N(R_{y1b})R_{y1a}, —[CH₂]ᵣ—N(R_{y1b})C(O)R_{y1a}, —[CH₂]ᵣ—N(R_{y1c})—C(O)—N(R_{y1b})R_{y1a}, —[CH₂]ᵣ—S(O)ₚR_{y1a} (where p is 0, 1 or 2), —[CH₂]ᵣ—SO₂N(R_{y1b})R_{y1a}, or —[CH₂]ᵣ—N(R_{y1b})SO₂R_{y1a};
wherein r is 0, 1, 2 or 3;
R_{y1} is hydrogen or (1-4C)alkyl optionally substituted by halo, hydroxy, amino or cyano; and
R_{y1b} and R_{y1c} are hydrogen or (1-2C)alkyl;
(ii) a group of the formula III: 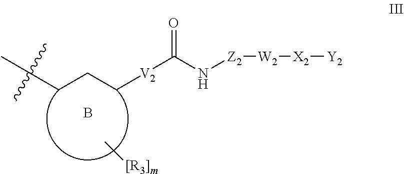
wherein:
Ring B is selected from phenyl or a 5- or 6-membered heteroaryl;
m is 0, 1 or 2;
each R₃ group, when present, is selected from: halo, nitro, cyano, R₃ₐ, —[CH₂]_{q1}—NR₃ₐR_{3b}, —[CH₂]_{q1}—OR₃ₐ, —[CH₂]_{q1}—C(O)R₃ₐ, —[CH₂]_{q1}—C(O)OR₃ₐ, —[CH₂]_{q1}—OC(O)R₃ₐ, —[CH₂]_{q1}—C(O)N(R_{3b})R₃ₐ, —[CH₂]_{q1}—N(R_{3b})C(O)R₃ₐ, —[CH₂]_{q1}—N(R_{3c})—C(O)—N(R_{3b})R₃ₐ, —[CH₂]_{q1}—S(O)ₚR₃ₐ (where p is 0, 1 or 2), —[CH₂]_{q1}—SO₂N(R_{3b})R₃ₐ, —[CH₂]_{q1}—N(R_{3b})SO₂R₃ₐ;
wherein q1 is 0, 1, 2 or 3;
R₃ₐ is hydrogen or (1-4C)alkyl optionally substituted by halo, hydroxy, amino or cyano; and
R_{3b} and R_{3c} are hydrogen or (1-2C)alkyl;
V₂ is selected from —C(Rᵥ₂ₐR_{v2b})C(R_{v2c}R_{v2d})— or —C(Rᵥ₂ₐ)═C(R_{2c})—, wherein Rᵥ₂ₐ, R_{v2b}, R_{v2c} and R_{v2d} are each independently selected from hydrogen or methyl or Rᵥ₂ₐ and R_{v2c} are linked to form a cyclopropyl ring;
Z₂ is a 4C alkylene linker which is optionally substituted by one or more fluoro atoms;
W₂ is a carbocyclic, aryl, heteroaryl or heterocyclic ring which is optionally substituted by halo, nitro, cyano, R_{w2a}, —[CH₂]ₛ—NR_{w2a}R_{w2b}, —[CH₂]ₛ—OR_{w2a}, —[CH₂]ₛ—C(O)R_{w2a}, —[CH₂]ₛ—C(O)OR_{w2a}, —[CH₂]ₛ—OC(O)R_{w2a}, —[CH₂]ₛ—C(O)N(R_{w2b})R_{w2a}, —[CH₂]ₛ—N(R_{w2b})C(O)R_{w2a}, —[CH₂]ₛ—N(R_{w2c})—C(O)—N(R_{w2b})R_{w2a}, —[CH₂]ₛ—S(O)ₚR_{w2a} (where p is 0, 1 or 2), —[CH₂]ₛ—SO₂N(R_{w2b})R_{w2a}, or —[CH₂]ₛ—N(R_{w2b})SO₂R_{w2a}; wherein
s is 0, 1, 2 or 3;
R_{w2a} is hydrogen or (1-4C)alkyl optionally substituted by halo, hydroxy, amino or cyano; and
R_{w2b} and R_{w2e} are hydrogen or (1-2C)alkyl;
X₂ is a linker group of the formula:
—[CH₂]ₘ₁-L₂-[CH₂]ₘ₂—
wherein
m1 and m2 are selected from 0 or 1;
L₂ is selected from —O—, —S—, —SO—, —SO₂—, —N(Rₓ₂ₐ)—, —C(O)—, —C(O)O—, —OC(O)—, —C(O)N(Rₓ₂ₐ)—, —N(Rₓ₂ₐ)C(O)—, —N(R_{x2b})C(O)N(Rₓ₂ₐ)—, —N(Rₓ₂ₐ)C(O)O—, —OC(O)N(Rₓ₂ₐ)—, —S(O)₂N(Rₓ₂ₐ), N(Rₓ₂ₐ)SO₂, or C(O)N(Rₓ₂ₐ)SO₂, or —SO₂N(Rₓ₂ₐ)C(O)—; and wherein Rₓ₂ₐ and R_{x2b} are each independently selected from hydrogen or (1-2C)alkyl;
Y₂ is selected from a carbocyclic, aryl, heteroaryl or heterocyclic ring which is optionally substituted by halo, nitro, cyano, R_{y2a}, —[CH₂]ₜ—NR_{y2a}R_{y2b}, —[CH₂]ₜ—OR_{y2a}, —[CH₂]ₜ—C(O)R_{y2a}, —[CH₂]ₜ—C(O)OR_{y2a}, —[CH₂]ₜ—OC(O)R_{y2a}, —[CH₂]ₜ—C(O)N(R_{y2b})R_{y2a}, —[CH₂]ₜ—N(R_{y2b})C(O)R_{y2a}, —[CH₂]ₜ—N(R_{y2c})—C(O)—N(R_{y2b})R_{y2a}, —[CH₂]ₜ—S(O)ₚR_{y2a} (where p is 0, 1 or 2), —[CH₂]ₜ—SO₂N(R_{y2b})R_{y2a}, or —[CH₂]ₜ—N(R_{y2b})SO₂R_{y2a};
wherein t is 0, 1, 2 or 3;
R_{y2a} is hydrogen or (1-4C)alkyl optionally substituted by halo, hydroxy, amino or cyano; and
R_{y2b} and R_{y2} are hydrogen or (1-2C)alkyl;
(iii) a group of the formula IV: 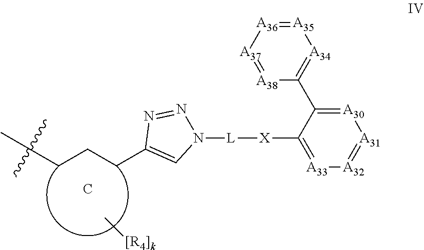
wherein:
Ring C is selected from phenyl, a 5- or 6-membered heteroaryl;
k is 0, 1 or 2;
each R₄ group, when present, is selected from: halo, nitro, cyano, R₄ₐ, —[CH₂]_{q2}—NR₄ₐR_{4b}, —[CH₂]_{q2}—OR₄ₐ, —[CH₂]_{q2}—C(O)R₄ₐ, —[CH₂]_{q2}—C(O)OR₄ₐ, —[CH₂]_{q2}—OC(O)R₄ₐ, —[CH₂]_{q2}—C(O)N(R_{4b})R₄ₐ, —[CH₂]_{q2}—N(R_{4b})C(O)R₄ₐ, —[CH₂]_{q2}—N(R_{4c})—C(O)—N(R_{4b})R₄ₐ, —[CH₂]_{q2}—S(O)ₚR₄ₐ (where p is 0, 1 or 2), —[CH₂]_{q2}—SO₂N(R_{4b})R₄ₐ, —[CH₂]_{q2}—N(R_{4b})SO₂R₄ₐ;
wherein q2 is 0, 1, 2 or 3;
R₄ₐ is hydrogen or (1-4C)alkyl optionally substituted by halo, hydroxy, amino or cyano; and
R_{4b} and R_{4c} are hydrogen or (1-2C)alkyl;
L is a 6C alkylene linker which is optionally substituted by one or more fluoro atoms;
X is selected from —O—, —CRₓₐR_{xb}—, —S—, —SO—, —SO₂—, —N(Rₓₐ)—, —C(O)—; and
wherein Rₓₐ and R_{xb} are each independently selected from hydrogen or methyl;
A₃₀ and A₃₁ are selected from CH, N or C—F;
A₃₂ and A₃₃ are selected from CH or N;
with the proviso that only one or two of A₃₀, A₃₁, A₃₂ and A₃₃ can be N;
A₃₄ and A₃₅ are selected from CH, N or C—R₃₀;
A₃₆, A₃₇ and A₃₈ are selected from CH or N;
with the proviso that only one or two of A₃₄, A₃₅, A₃₆, A₃₇ and A₃₈ can be N;
and wherein R₃₀ is selected from halo, nitro, cyano, R₃₀ₐ, —[CH₂]ₜ—NR₃₀ₐR_{30b}, —[CH₂]ₜ—OR₃₀ₐ, —[CH₂]ₜ—C(O)R₃₀ₐ, —[CH₂]ₜ—C(O)OR₃₀ₐ, —[CH₂]ₜ—OC(O)R₃₀ₐ, —[CH₂]ₜ—C(O)N(R_{30b})R₃₀ₐ, —[CH₂]ₜ—N(R_{30b})C(O)R₃₀ₐ, —[CH₂]ₜ—N(R_{30c})—C(O)—N(R_{30b})R₃₀ₐ, —[CH₂]ₜ—S(O)ₚR₃₀ₐ (where p is 0, 1 or 2), —[CH₂]ₜ—SO₂N(R_{30b})R₃₀ₐ, or —[CH₂]ₜ—N(R_{30b})SO₂R₃₀ₐ; wherein
t is 0, 1, 2 or 3;
R₃₀ₐ is hydrogen or (1-4C)alkyl optionally substituted by halo, hydroxy, amino or cyano; and
R_{30b} and R_{30c} are hydrogen or (1-2C)alkyl;
(iv) a group of the formula V or VI: 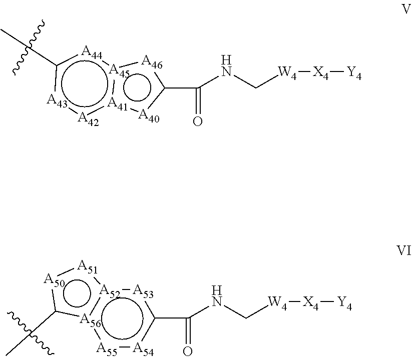
wherein:
A₄₀ is selected from NH, NMe or O;
A₄₂, A₄₃, A₄₄ and A₄₆ are each independently selected from CH, N or CR₂;
A₄₁ and A₄₅ are each independently selected from C or N;
with the proviso that:
(iv) only up to three of A₄₀, A₄₁, A₄₂, A₄₃, A₄₄, A₄₅ and A₄₆ are N;
(v) A₄₁ and A₄₅ cannot both be N;
(vi) only one or two of A₄₀, A₄₂, A₄₃, A₄₄ and A₄₆ can be CR₂;
A₅₁ is selected from NH, NMe, CH or CR₂;
A₅₀, A₅₃, A₅₄ and A₅₅ are each independently selected from CH, N or CR₂;
A₅₂ and A₅₆ are each independently selected from C or N;
with the proviso that:
(iv) only up to three of A₅₀, A₅₁, A₅₂, A₅₃, A₅₄, A₅₅ and A₅₆ are N;
(v) A₅₂ and A₅₆ cannot both be N;
(vi) only one or two of A₅₀, A₅₁, A₅₃, A₅₄ and A₅₅ can be CR₂;
wherein R₂ is as defined above;
W₄ is: 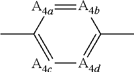
wherein A₄ₐ, A_{4b}, A_{4c} or A_{4d} are selected from CH, N or C—F, with the provisos that:
only one or two of A₄ₐ, A_{4b}, A_{4c} or A_{4d} can be N; and
only one or two of A₄ₐ, A_{4b}, A_{4c} or A_{4d} can be C—F;
X₄ is a linker group of the formula:
—[CH₂]ⱼ₁-L₄-[CH₂]ⱼ₂—
wherein
j1 and j2 are selected from 0 or 1;
L₄ is selected from —O—, —S—, —SO—, —SO₂—, —N(Rₓ₄ₐ)—, —C(O)—, —C(O)O—, —OC(O)—, —C(O)N(Rₓ₄ₐ)—, —N(Rₓ₄ₐ)C(O)—, —N(R_{x4b})C(O)N(Rₓ₄ₐ)—, —N(Rₓ₄ₐ)C(O)O—, —OC(O)N(Rₓ₄ₐ)—, —S(O)₂N(Rₓ₄ₐ), —N(Rₓ₄ₐ)SO₂—, or —C(O)N(Rₓ₄ₐ)SO₂—, or —SO₂N(Rₓ₄ₐ)C(O)—; and wherein Rₓ₄ₐ and R_{x4b} are each independently selected from hydrogen or (1-2C)alkyl;
Y₄ is selected from a carbocyclic, aryl, heteroaryl or heterocyclic ring which is optionally substituted by halo, nitro, cyano, R_{y4a}, —[CH₂]ᵤ—NR_{y4a}R_{y4b}, —[CH₂]—OR_{y4a}, —[CH₂]ᵤ—C(O)R_{y4a}, —[CH₂]ᵤ—C(O)OR_{y4a}, —[CH₂]ᵤ—OC(O)R_{y4a}, —[CH₂]ᵤ—C(O)N(R_{y4b})R_{y4a}, —[CH₂]ᵤ—N(R_{y4b})C(O)R_{y4a}, —[CH₂]ᵤ—N(R_{y4c})—C(O)—N(R_{y4b})R_{y4a}, —[CH₂]ᵤ—S(O)ₚR_{y4a} (where p is 0, 1 or 2), —[CH₂]ᵤ—SO₂N(R_{y4b})R_{y4a}, or —[CH₂]ᵤ—N(R_{y4b})SO₂R_{y4a};
wherein u is 0, 1, 2 or 3;
R_{y4} is hydrogen or (1-4C)alkyl optionally substituted by halo, hydroxy, amino or cyano; and
R_{y4b} and R_{y4c} are hydrogen or (1-2C)alkyl.

2. A compound according to claim 1, or a pharmaceutically acceptable salt thereof wherein R₁ is selected from:
(i) a group of the formula II: 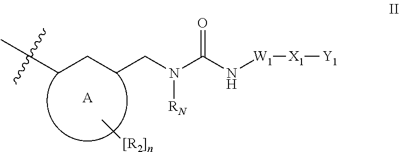
wherein:
Ring A is selected from phenyl or a 5- or 6-membered heteroaryl;
n is 0, 1 or 2;
each R₂ group, when present, is selected from: halo, nitro, cyano, R₂ₐ, —[CH₂]_{q}—NR₂ₐR_{2b}, —[CH₂]_{q}—OR₂ₐ, —[CH₂]_{q}—C(O)R₂ₐ, —[CH₂]_{q}—C(O)OR₂ₐ, —[CH₂]_{q}—OC(O)R₂ₐ, —[CH₂]_{q}—C(O)N(R_{2b})R₂ₐ, —[CH₂]_{q}—N(R_{2b})C(O)R₂ₐ, —[CH₂]_{q}—N(R_{2c})—C(O)—N(R_{2b})R₂ₐ;
wherein q is 0, 1 or 2;
R₂ₐ is hydrogen or (1-4C)alkyl optionally substituted by halo, hydroxy, amino or cyano; and
R_{2b} and R_{2c} are hydrogen or (1-2C)alkyl;
R_{N} is selected from hydrogen or methyl;
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, (1-2C)alkyl, (1-2C)haloalkyl, cyano or amino;
W₁ is: 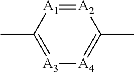
wherein A₁, A₂, A₃ or A₄ are selected from CH, N or C—F, with the provisos that:
only one or two of A₁, A₂, A₃ or A₄ can be N; and
only one or two of A₁, A₂, A₃ or A₄ can be C—F;
X₁ is a linker group of the formula:
—[CH₂]ₙ₁-L₁-[CH₂]ₙ₂—
wherein
n1 and n2 are selected from 0 or 1;
L₁ is selected from —O—, —S—, —SO—, —SO₂—, —N(Rₓ₁ₐ)—, —C(O)—, —C(O)O—, —OC(O)—, —C(O)N(Rₓ₁ₐ)—, —N(Rₓ₁ₐ)C(O)—, —N(R_{x1b})C(O)N(Rₓ₁ₐ)—, —N(Rₓ₁ₐ)C(O)O— or —OC(O)N(Rₓ₁ₐ)—; and wherein Rₓ₁ₐ and R_{x1b} are each independently selected from hydrogen or (1-2C)alkyl;
Y₁ is selected from a carbocyclic, aryl, heteroaryl or heterocyclic ring which is optionally substituted by halo, nitro, cyano, R_{y1a}, —[CH₂]ᵣ—NR_{y1a}R_{y1b}, —[CH₂]ᵣ—OR_{y1a}, —[CH₂]ᵣ—C(O)R_{y1a}, —[CH₂]ᵣ—C(O)OR_{y1a}, —[CH₂]ᵣ—OC(O)R_{y1a}, —[CH₂]ᵣ—C(O)N(R_{y1b})R_{y1a}, —[CH₂]ᵣ—N(R_{y1b})C(O)R_{y1a} or —[CH₂]ᵣ—N(R_{y1c})—C(O)—N(R_{y1b})R_{y1a};
wherein r is 0, 1 or 2;
R_{y1} is hydrogen or (1-4C)alkyl optionally substituted by halo, hydroxy, amino or cyano; and
R_{y1b} and R_{y1c} are hydrogen or (1-2C)alkyl;
(ii) a group of the formula III: 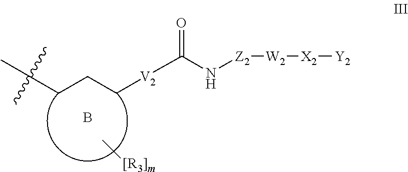
wherein:
Ring B is selected from phenyl or a 5- or 6-membered heteroaryl;
m is 0, 1 or 2;
each R₃ group, when present, is selected from: halo, nitro, cyano, R₃ₐ, —[CH₂]_{q1}—NR₃ₐR_{3b}, —[CH₂]_{q1}—OR₃ₐ, —[CH₂]_{q1}—C(O)R₃ₐ, —[CH₂]_{q1}—C(O)OR₃ₐ, —[CH₂]_{q1}—OC(O)R₃ₐ, —[CH₂]_{q1}—C(O)N(R_{3b})R₃ₐ, —[CH₂]_{q1}—N(R_{3b})C(O)R₃ₐ or —[CH₂]_{q1}—N(R_{3c})—C(O)—N(R_{3b})R₃ₐ;
wherein q1 is 0, 1 or 2;
R₃ₐ is hydrogen or (1-4C)alkyl optionally substituted by halo, hydroxy, amino or cyano; and
R_{3b} and R_{3c} are hydrogen or (1-2C)alkyl;
V₂ is selected from —C(Rᵥ₂ₐR_{v2b})C(R_{2c}R_{v2d})— or —C(Rᵥ₂ₐ)═C(R_{v2c})—, wherein Rᵥ₂ₐ, R_{v2b}, R_{v2c} and R_{v2d} are each independently selected from hydrogen or methyl or Rᵥ₂ₐ and R_{v2c} are linked to form a cyclopropyl ring;
Z₂ is a 4C alkylene linker which is optionally substituted by one or more fluoro atoms;
W₂ is a carbocyclic, aryl, heteroaryl or heterocyclic ring which is optionally substituted by halo, nitro, cyano, R_{w2a}, —[CH₂]ₛ—NR_{w2a}R_{w2b}, —[CH₂]ₛ—OR_{w2a}, —[CH₂]ₛ—C(O)R_{w2a}, —[CH₂]ₛ—C(O)OR_{w2a}, —[CH₂]ₛ—OC(O)R_{w2a}, —[CH₂]ₛ—C(O)N(R_{w2b})R_{w2a}, —[CH₂]ₛ—N(R_{w2b})C(O)R_{w2a} or —[CH₂]ₛ—N(R_{w2c})—C(O)—N(R_{w2b})R_{w2a}; wherein
s is 0, 1 or 2;
R_{w2a} is hydrogen or (1-4C)alkyl optionally substituted by halo, hydroxy, amino or cyano; and
R_{w2b} is hydrogen or (1-2C)alkyl;
X₂ is a linker group of the formula:
—[CH₂]ₘ₁-L₂-[CH₂]ₘ₂—
wherein
m1 and m2 are selected from 0 or 1;
L₂ is selected from —O—, —S—, —SO—, —SO₂—, —N(Rₓ₂ₐ)—, —C(O)—, —C(O)O—, —OC(O)—, —C(O)N(Rₓ₂ₐ)—, —N(Rₓ₂ₐ)C(O)—, —N(R_{x2b})C(O)N(Rₓ₂ₐ)—, —N(Rₓ₂ₐ)C(O)O— or —OC(O)N(Rₓ₂ₐ)—; and wherein Rₓ₂ₐ and R_{x2b} are each independently selected from hydrogen or (1-2C)alkyl;
Y₂ is selected from a carbocyclic, aryl, heteroaryl or heterocyclic ring which is optionally substituted by halo, nitro, cyano, R_{y2a}, —[CH₂]ₜ—NR_{y2a}R_{y2b}, —[CH₂]ₜ—OR_{y2a}, —[CH₂]ₜ—C(O)R_{y2a}, —[CH₂]ₜ—C(O)OR_{y2a}, —[CH₂]ₜ—OC(O)R_{y2a}, —[CH₂]ₜ—C(O)N(R_{y2b})R_{y2a}, —[CH₂]ₜ—N(R_{y2b})C(O)R_{y2a} or —[CH₂]ₜ—N(R_{y2c})—C(O)—N(R_{y2b})R_{y2a};
wherein t is 0, 1 or 2;
R_{y2a} is hydrogen or (1-4C)alkyl optionally substituted by halo, hydroxy, amino or cyano; and
R_{y2b} and R_{y2c} are hydrogen or (1-2C)alkyl;
(iii) a group of the formula IV: 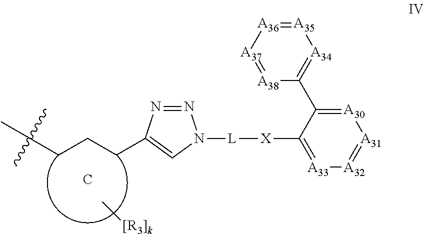
wherein:
Ring C is selected from phenyl, a 5- or 6-membered heteroaryl;
k is 0, 1 or 2;
each R₄ group, when present, is selected from: halo, nitro, cyano, R₄ₐ, —[CH₂]_{q2}—NR₄ₐR_{4b}, —[CH₂]_{q2}—OR₄ₐ, —[CH₂]_{q2}—C(O)R₄ₐ, —[CH₂]_{q2}—C(O)OR₄ₐ, —[CH₂]_{q2}—OC(O)R₄ₐ, —[CH₂]_{q2}—C(O)N(R_{4b})R₄ₐ or —[CH₂]_{q2}—N(R_{4b})C(O)R₄ₐ, —[CH₂]_{q2}—N(R_{4c})—C(O)—N(R_{4b})R₄ₐ;
wherein q2 is 0, 1, 2 or 3;
R₄ₐ is hydrogen or (1-4C)alkyl optionally substituted by halo, hydroxy, amino or cyano; and
R_{4b} and R₄ₑ are hydrogen or (1-2C)alkyl;
L is a 6C alkylene linker which is optionally substituted by one or more fluoro atoms;
X is selected from —O—, —CRₓₐR_{xb}—, —S—, —SO—, —SO₂—, —N(Rₓₐ)—, —C(O)—; and
wherein Rₓₐ and R_{xb} are each independently selected from hydrogen or methyl;
A₃₀ and A₃₁ are selected from CH, N or C—F;
A₃₂ and A₃₃ are selected from CH or N;
with the proviso that only one or two of A₃₀, A₃₁, A₃₂ and A₃₃ can be N;
A₃₄ and A₃₅ are selected from CH, N or C—R₃₀;
A₃₆, A₃₇ and A₃₈ are selected from CH or N;
with the proviso that only one or two of A₃₄, A₃₅, A₃₆, A₃₇ and A₃₈ can be N;
and wherein R₃₀ is selected from halo, nitro, cyano, R₃₀ₐ, —[CH₂]ₜ—NR₃₀ₐR_{30b}, —[CH₂]ₜ—OR₃₀ₐ, —[CH₂]ₜ—C(O)R₃₀ₐ, —[CH₂]ₜ—C(O)OR₃₀ₐ, —[CH₂]ₜ—OC(O)R₃₀ₐ, —[CH₂]ₜ—C(O)N(R_{30b})R₃₀ₐ, —[CH₂]ₜ—N(R_{30b})C(O)R₃₀ₐ or —[CH₂]ₜ—N(R_{30c})—C(O)—N(R_{30b})R₃₀ₐ; wherein
t is 0, 1 or 2;
R₃₀ₐ is hydrogen or (1-4C)alkyl optionally substituted by halo, hydroxy, amino or cyano; and
R_{30b} and R_{30c} are hydrogen or (1-2C)alkyl;
(iv) a group of the formula V or VI: 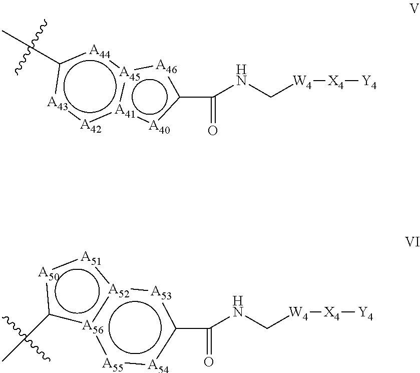
wherein:
A₄₀ is selected from NH, NMe or O;
A₄₂, A₄₃, A₄₄ and A₄₆ are each independently selected from CH, N or CR₂;
A₄₁ and A₄₅ are each independently selected from C or N;
with the proviso that:
(vii) only up to three of A₄₀, A₄₁, A₄₂, A₄₃, A₄₄, A₄₅ and A₄₆ are N;
(viii) A₄₁ and A₄₅ cannot both be N;
(ix) only one or two of A₄₀, A₄₂, A₄₃, A₄₄ and A₄₆ can be CR₂;
A₅₁ is selected from NH, NMe, CH or CR₂;
A₅₀, A₅₃, A₅₄ and A₅₅ are each independently selected from CH, N or CR₂;
A₅₂ and A₅₆ are each independently selected from C or N;
with the proviso that:
(vii) only up to three of A₅₀, A₅₁, A₅₂, A₅₃, A₅₄, A₅₅ and A₅₆ are N;
(viii) A₅₂ and A₅₆ cannot both be N;
(ix) only one or two of A₅₀, A₅₁, A₅₃, A₅₄ and A₅₅ can be C R₂;
wherein R₂ is as defined above;
W₄ is: 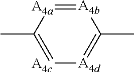
wherein A₄ₐ, A_{4b}, A_{4c} or A_{4d} are selected from CH, N or C—F, with the provisos that:
only one or two of A₄ₐ, A_{4b}, A_{4c} or A_{4d} can be N; and
only one or two of A₄ₐ, A_{4b}, A_{4c} or A_{4d} can be C—F;
X₄ is a linker group of the formula:
—[CH₂]ⱼ₁-L₄-[CH₂]ⱼ₂—
wherein
j1 and j2 are selected from 0 or 1;
L₄ is selected from —O—, —S—, —SO—, —SO₂—, —N(Rₓ₄ₐ)—, —C(O)—, —C(O)O—, —OC(O)—, —C(O)N(Rₓ₄ₐ)—, —N(Rₓ₄ₐ)C(O)—, —N(R_{x4b})C(O)N(Rₓ₄ₐ)—, —N(Rₓ₄ₐ)C(O)O— or —OC(O)N(Rₓ₄ₐ)—; and wherein Rₓ₄ₐ and R_{x4b} are each independently selected from hydrogen or (1-2C)alkyl;
Y₄ is selected from a carbocyclic, aryl, heteroaryl or heterocyclic ring which is optionally substituted by halo, nitro, cyano, R_{y4a}, —[CH₂]ᵤ—NR_{y4a}R_{y4b}, —[CH₂]ᵤ—OR_{y4a}, —[CH₂]ᵤ—C(O)R_{y4a}, —[CH₂]ᵤ—C(O)OR_{y4a}, —[CH₂]ᵤ—OC(O)R_{y4a}, —[CH₂]ᵤ—C(O)N(R_{y4b})R_{y4a}, —[CH₂]ᵤ—N(R_{y4b})C(O)R_{y4a} or —[CH₂], —N(R_{y4c})—C(O)—N(R_{y4e})R_{y4a};
wherein u is 0, 1 or 2;
R_{y4} is hydrogen or (1-4C)alkyl optionally substituted by halo, hydroxy, amino or cyano; and
R_{y4b} and R_{y4c} are hydrogen or (1-2C)alkyl.

3. A compound according to claim 1 or 2, or a pharmaceutically acceptable salt thereof, wherein R₁ is selected from:
(i) a group of the formula II: 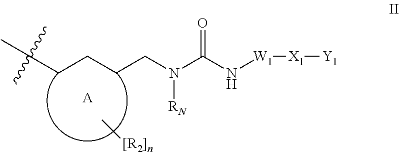
wherein:
Ring A is selected from phenyl or a 5- or 6-membered heteroaryl;
n is 0, 1 or 2;
each R₂ group, when present, is selected from: halo, nitro, cyano, R₂ₐ, —[CH₂]_{q}—NR₂ₐR_{2b}, —[CH₂]_{q}—OR₂ₐ, —[CH₂]_{q}—C(O)R₂ₐ, —[CH₂]_{q}—C(O)OR₂ₐ or —[CH₂]_{q}—OC(O)R₂ₐ;
wherein q is 0, 1 or 2;
R₂ₐ is hydrogen or (1-2C)alkyl optionally substituted by halo, hydroxy, amino or cyano; and
R_{2b} is hydrogen or (1-2C)alkyl;
R_{N} is selected from hydrogen or methyl;
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, (1-2C)alkyl, (1-2C)haloalkyl, cyano or amino;
W₁ is: 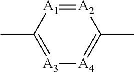
wherein A₁, A₂, A₃ or A₄ are selected from CH, N or C—F, with the provisos that:
only one or two of A₁, A₂, A₃ or A₄ can be N; and
only one or two of A₁, A₂, A₃ or A₄ can be C—F;
X₁ is a linker group of the formula:
—[CH₂]ₙ₁-L₁-[CH₂]ₙ₂—
wherein
n1 and n2 are selected from 0 or 1;
L₁ is selected from —O—, —S—, —SO—, —SO₂—, —N(Rₓ₁ₐ)—, —C(O)—, —C(O)O—, —OC(O)—, —C(O)N(Rₓ₁ₐ)— or —N(Rₓ₁ₐ)C(O)—; and wherein Rₓ₁ₐ and R_{x1b} are each independently selected from hydrogen or (1-2C)alkyl;
Y₁ is selected from a carbocyclic, aryl, heteroaryl or heterocyclic ring which is optionally substituted by halo, nitro, cyano, R_{y1a}, —[CH₂]ᵣ—NR_{y1a}R_{y1b}, —[CH₂]ᵣ—OR_{y1a}, —[CH₂]ᵣ—C(O)R_{y1a}, —[CH₂]ᵣ—C(O)OR_{y1a} or —[CH₂]ᵣ—OC(O)R_{y1a};
wherein r is 0 or 1;
R_{y1a} is hydrogen or (1-2C)alkyl optionally substituted by halo, hydroxy, amino or cyano; and
R_{y1b} and R_{y1c} are hydrogen or (1-2C)alkyl;
(ii) a group of the formula III: 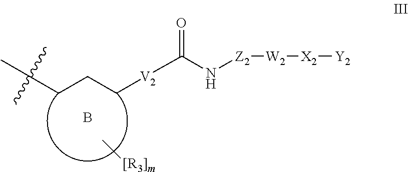
wherein:
Ring B is selected from phenyl or a 5- or 6-membered heteroaryl;
m is 0, 1 or 2;
each R₃ group, when present, is selected from: halo, nitro, cyano, R₃ₐ, —[CH₂]_{q}1-NR₃ₐR_{3b}, —[CH₂]_{q}1-OR₃ₐ, —[CH₂]_{q}1-C(O)R₃ₐ, —[CH₂]_{q1}—C(O)OR₃ₐ or —[CH₂]_{q1}—OC(O)R₃ₐ;
wherein q1 is 0, 1 or 2;
R₃ₐ is hydrogen or (1-2C)alkyl optionally substituted by halo, hydroxy, amino or cyano; and
R_{3b} is hydrogen or (1-2C)alkyl;
V₂ is selected from —C(Rᵥ₂ₐR_{v2b})C(R_{v2c}R_{v2d})— or —C(Rᵥ₂ₐ)═C(R_{2c})—, wherein Rᵥ₂ₐ, R_{v2b}, R_{v2c} and R_{v2d} are each independently selected from hydrogen or methyl or Rᵥ₂ₐ and R_{v2c} are linked to form a cyclopropyl ring;
Z₂ is a 4C alkylene linker which is optionally substituted by one or more fluoro atoms;
W₂ is a carbocyclic, aryl, heteroaryl or heterocyclic ring which is optionally substituted by halo, nitro, cyano, R_{w2a}, —[CH₂]ₛ—NR_{w2a}R_{w2b}, —[CH₂]ₛ—OR_{w2a}, —[CH₂]ₛ—C(O)R_{w2a}, —[CH₂]ₛ—C(O)OR_{w2a} or —[CH₂]ₛ—OC(O)R_{w2a}; wherein
s is 0, 1 or 2;
R_{w2a} is hydrogen or (1-4C)alkyl optionally substituted by halo, hydroxy, amino or cyano; and
R_{w2b} and R_{w2e} are hydrogen or (1-2C)alkyl;
X₂ is a linker group of the formula:
—[CH₂]ₘ₁-L₂-[CH₂]ₘ₂—
wherein
m1 and m2 are selected from 0 or 1;
L₂ is selected from —O—, —S—, —SO—, —SO₂—, —N(Rₓ₂ₐ)—, —C(O)—, —C(O)O—, —OC(O)—, —C(O)N(Rₓ₂ₐ)— or —N(Rₓ₂ₐ)C(O)—, and wherein Rₓ₂ₐ and R_{x2b} are each independently selected from hydrogen or (1-2C)alkyl;
Y₂ is selected from a carbocyclic, aryl, heteroaryl or heterocyclic ring which is optionally substituted by halo, nitro, cyano, R_{y2a}, —[CH₂]ₜ—NR_{y2a}R_{y2b}, —[CH₂]ₜ—OR_{y2a}, —[CH₂]ₜ—C(O)R_{y2a}, —[CH₂]ₜ—C(O)OR_{y2a} or —[CH₂]ₜ—OC(O)R_{y2a};
wherein t is 0 or 1;
R_{y2a} is hydrogen or (1-2C)alkyl optionally substituted by halo, hydroxy, amino or cyano; and
R_{y2b} is hydrogen or (1-2C)alkyl;
(iii) a group of the formula IV: 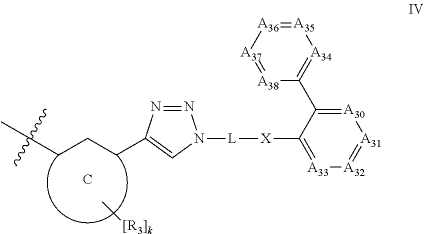
wherein:
Ring C is selected from phenyl, a 5- or 6-membered heteroaryl;
k is 0, 1 or 2;
each R₄ group, when present, is selected from: halo, nitro, cyano, R₄ₐ, —[CH₂]_{q2}—NR₄ₐR_{4b}, —[CH₂]_{q2}—OR₄ₐ, —[CH₂]_{q2}—C(O)R₄ₐ, —[CH₂]_{q2}—C(O)OR₄ₐ or —[CH₂]_{q2}—OC(O)R₄ₐ;
wherein q2 is 0, 1, 2 or 3;
R₄ₐ is hydrogen or (1-4C)alkyl optionally substituted by halo, hydroxy, amino or cyano; and
R_{4b} is hydrogen or (1-2C)alkyl;
L is a 6C alkylene linker which is optionally substituted by one or more fluoro atoms;
X is selected from —O—, —CRₓₐR_{xb}—, —S—, —SO—, —SO₂—, —N(Rₓₐ)—, —C(O)—; and
wherein Rₓₐ and R_{xb} are each independently selected from hydrogen or methyl;
A₃₀ and A₃₁ are selected from CH, N or C—F;
A₃₂ and A₃₃ are selected from CH or N;
with the proviso that only one or two of A₃₀, A₃₁, A₃₂ and A₃₃ can be N;
A₃₄ and A₃₅ are selected from CH, N or C—R₃₀;
A₃₆, A₃₇ and A₃₈ are selected from CH or N;
with the proviso that only one or two of A₃₄, A₃₅, A₃₆, A₃₇ and A₃₈ can be N;
and wherein R₃₀ is selected from halo, nitro, cyano, R₃₀ₐ, —[CH₂]ₜ—NR₃₀ₐR_{30b}, —[CH₂]ₜ—OR₃₀ₐ, —[CH₂]ₜ—C(O)R₃₀ₐ, —[CH₂]ₜ—C(O)OR₃₀ₐ or —[CH₂]ₜ—OC(O)R₃₀ₐ; wherein
t is 0, 1 or 2;
R₃₀ₐ is hydrogen or (1-4C)alkyl optionally substituted by halo, hydroxy, amino or cyano; and
R_{30b} and R_{30c} are hydrogen or (1-2C)alkyl;
(iv) a group of the formula V or VI: 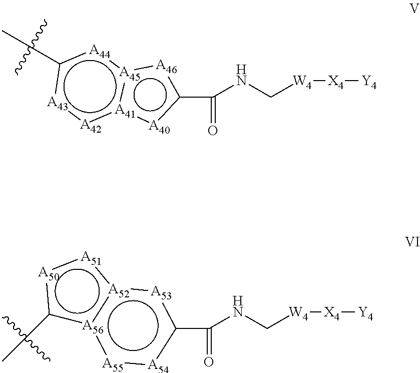
wherein:
A₄₀ is selected from NH, NMe or O;
A₄₂, A₄₃, A₄₄ and A₄₆ are each independently selected from CH, N or CR₂;
A₄₁ and A₄₅ are each independently selected from C or N;
with the proviso that:
(i) only up to three of A₄₀, A₄₁, A₄₂, A₄₃, A₄₄, A₄₅ and A₄₆ are N;
(ii) A₄₁ and A₄₅ cannot both be N;
(iii) only one or two of A₄₀, A₄₂, A₄₃, A₄₄ and A₄₆ can be CR₂;
A₅₁ is selected from NH, NMe, CH or CR₂;
A₅₀, A₅₃, A₅₄ and A₅₅ are each independently selected from CH, N or CR₂;
A₅₂ and A₅₆ are each independently selected from C or N;
with the proviso that:
(i) only up to three of A₅₀, A₅₁, A₅₂, A₅₃, A₅₄, A₅₅ and A₅₆ are N;
(ii) A₅₂ and A₅₆ cannot both be N;
(iii) only one or two of A₅₀, A₅₁, A₅₃, A₅₄ and A₅₅ can be CR₂;
wherein R₂ is as defined above;
W₄ is: 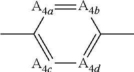
wherein A₄ₐ, A_{4b}, A_{4c} or A_{4d} are selected from CH, N or C—F, with the provisos that:
only one or two of A₄ₐ, A_{4b}, A_{4c} or A_{4d} can be N; and
only one or two of A₄ₐ, A_{4b}, A_{4c} or A_{4d} can be C—F;
X₄ is a linker group of the formula:
—[CH₂]ⱼ₁-L₄-[CH₂]ⱼ₂—
wherein
j1 and j2 are selected from 0 or 1;
L₄ is selected from —O—, —S—, —SO—, —SO₂—, —N(Rₓ₄ₐ)—, —C(O)—, —C(O)O—, —OC(O)—, —C(O)N(Rₓ₄ₐ)— or —N(Rₓ₄ₐ)C(O)—; and wherein Rₓ₄ₐ and R_{x4b} are each independently selected from hydrogen or (1-2C)alkyl;
Y₄ is selected from a carbocyclic, aryl, heteroaryl or heterocyclic ring which is optionally substituted by halo, nitro, cyano, R_{y4a}, —[CH₂]ᵤ—NR_{y4a}R_{y4b}, —[CH₂]ᵤ—OR_{y4a}, —[CH₂]ᵤ—C(O)R_{y4a}, —[CH₂]ᵤ—C(O)OR_{y4a} or —[CH₂]ᵤ—OC(O)R_{y4a},
wherein u is 0 or 1;
R_{y4a} is hydrogen or (1-2C)alkyl optionally substituted by halo, hydroxy, amino or cyano; and
R_{y4b} and R_{y4c} are hydrogen or (1-2C)alkyl.

4. A compound according to any one of the preceding claims, or a pharmaceutically acceptable salt thereof, wherein R₁ is selected from:
(i) a group of the formula II: 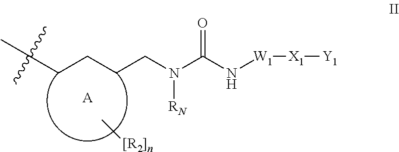
wherein:
Ring A is selected from phenyl or a 5- or 6-membered heteroaryl;
n is 0, 1 or 2;
each R₂ group, when present, is selected from: halo, nitro, cyano, R₂ₐ, —[CH₂]_{q}—NR₂ₐR_{2b}, —[CH₂]_{q}—OR₂ₐ, —[CH₂]_{q}—C(O)R₂ₐ;
wherein q is 0 or 1;
R₂ₐ is hydrogen or methyl; and
R_{2b} is hydrogen or methyl;
R_{N} is selected from hydrogen or methyl;
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, methyl, halomethyl, cyano or amino;
W₁ is: 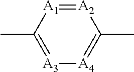
wherein A₁, A₂, A₃ or A₄ are selected from CH or C—F, with the proviso that:
only one or two of A₁, A₂, A₃ or A₄ can be C—F;
X₁ is a linker group of the formula:
—[CH₂]ₙ₁-L₁-[CH₂]ₙ₂—
wherein
n1 and n2 are selected from 0 or 1;
L₁ is selected from —O—, —S—, —SO—, —SO₂—, —N(Rₓ₁ₐ)— or —C(O)—; and wherein Rₓ₁ₐ is selected from hydrogen or methyl;
Y₁ is selected from a phenyl, heteroaryl or heterocyclic ring which is optionally substituted by halo, nitro, cyano, R_{y1a}, —NR_{y1a}R_{y1b}, —OR_{y1a} or —C(O)R_{y1a};
R_{y1a} is hydrogen or methyl; and
R_{y1b} is hydrogen or methyl;
(ii) a group of the formula III: 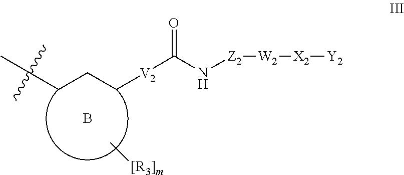
wherein:
Ring B is selected from phenyl or a 5- or 6-membered heteroaryl;
m is 0, 1 or 2;
each R₃ group, when present, is selected from: halo, nitro, cyano, R₃ₐ, —[CH₂]_{q1}—NR₃ₐR_{3b}, —[CH₂]_{q1}—OR₃ₐ or —[CH₂]_{q1}—C(O)R₃ₐ;
wherein q1 is 0 or 1;
R₃ₐ is hydrogen or methyl; and
R_{3b} is hydrogen or methyl;
V₂ is selected from —C(Rᵥ₂ₐR_{v2b})C(R_{v2c}R_{v2d})— or —C(Rᵥ₂ₐ)═C(R_{v2c})—, wherein Rᵥ₂ₐ, R_{v2b}, R_{v2c} and R_{v2d} are each independently selected from hydrogen or methyl or Rᵥ₂ₐ and R_{v2c} are linked to form a cyclopropyl ring;
Z₂ is a 4C alkylene linker which is optionally substituted by one or more fluoro atoms;
W₂ is a carbocyclic or heterocyclic ring which is optionally substituted by halo, nitro, cyano, R_{w2a}, —[CH₂]ₛ—NR_{w2a}R_{w2b}, —[CH₂]ₛ—OR_{w2a} or —[CH₂]ₛ—C(O)R_{w2a}; wherein
s is 0 or 1;
R_{w2a} is hydrogen or (1-2C)alkyl optionally substituted by halo, hydroxy, amino or cyano; and
R_{w2b} and R_{w2e} are hydrogen or (1-2C)alkyl;
X₂ is a linker group of the formula:
—[CH₂]ₘ₁-L₂-[CH₂]ₘ₂—
wherein
m1 and m2 are selected from 0 or 1;
L₂ is selected from —O—, —S—, —SO—, —SO₂—, —N(Rₓ₂ₐ)— or —C(O)— and wherein Rₓ₂ₐ is selected from hydrogen or methyl;
Y₂ is selected from a phenyl, heteroaryl or heterocyclic ring which is optionally substituted by halo, nitro, cyano, R_{y2a}, NR_{y2a}R_{y2b}, OR_{y2a} or C(O)R_{y2a};
R_{y2a} is hydrogen or methyl; and
R_{y2b} is hydrogen or methyl;
(iii) a group of the formula IV: 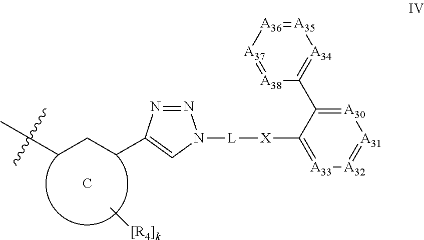
wherein:
Ring C is selected from phenyl, a 5- or 6-membered heteroaryl;
k is 0, 1 or 2;
each R₄ group, when present, is selected from: halo, nitro, cyano, R₄ₐ, —[CH₂]_{q2}—NR₄ₐR_{4b}, —[CH₂]_{q2}—OR₄ₐ, —[CH₂]_{q2}—C(O)R₄ₐ;
wherein q2 is 0 or 1;
R₄ₐ is hydrogen or methyl; and
R_{4b} is hydrogen or methyl;
L is a 6C alkylene linker which is optionally substituted by one or more fluoro atoms;
X is selected from —O—, —CRₓₐR_{xb}—, —S—, —SO—, —SO₂—, —N(Rₓₐ)—, —C(O)—; and
wherein Rₓₐ and R_{xb} are each independently selected from hydrogen or methyl;
A₃₀ and A₃₁ are selected from CH, N or C—F;
A₃₂ and A₃₃ are selected from CH or N;
with the proviso that only one or two of A₃₀, A₃₁, A₃₂ and A₃₃ can be N;
A₃₄ and A₃₅ are selected from CH, N or C—R₃₀;
A₃₆, A₃₇ and A₃₈ are selected from CH or N;
with the proviso that only one or two of A₃₄, A₃₅, A₃₆, A₃₇ and A₃₈ can be N;
and wherein R₃₀ is selected from halo, nitro, cyano, R₃₀ₐ, —NR₃₀ₐR_{30b}, —OR₃₀ₐ or —C(O)R₃₀ₐ; wherein
R₃₀ₐ is hydrogen or methyl; and
R_{30b} is hydrogen or methyl;
(iv) a group of the formula V or VI: 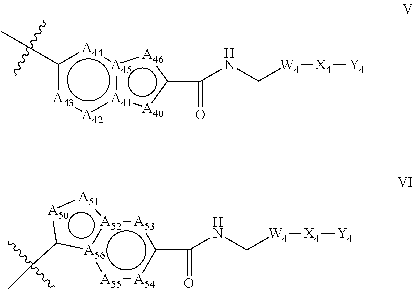
wherein:
A₄₀ is selected from NH, NMe or O;
A₄₂, A₄₃, A₄₄ and A₄₆ are each independently selected from CH, N or CR₂;
A₄₁ and A₄₅ are each independently selected from C or N;
with the proviso that:
(i) only up to three of A₄₀, A₄₁, A₄₂, A₄₃, A₄₄, A₄₅ and A₄₆ are N;
(ii) A₄₁ and A₄₅ cannot both be N;
(iii) only one or two of A₄₀, A₄₂, A₄₃, A₄₄ and A₄₆ can be CR₂;
A₅₁ is selected from NH, NMe, CH or CR₂;
A₅₀, A₅₃, A₅₄ and A₅₅ are each independently selected from CH, N or CR₂;
A₅₂ and A₅₆ are each independently selected from C or N;
with the proviso that:
(i) only up to three of A₅₀, A₅₁, A₅₂, A₅₃, A₅₄, A₅₅ and A₅₆ are N;
(ii) A₅₂ and A₅₆ cannot both be N;
(iii) only one or two of A₅₀, A₅₁, A₅₃, A₅₄ and A₅₅ can be CR₂;
wherein R₂ is as defined above;
W₄ is: 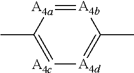
wherein A₄ₐ, A_{4b}, A_{4c} or A_{4d} are selected from CH or C—F, with the proviso that:
only one or two of A₄ₐ, A_{4b}, A_{4c} or A_{4d} can be C—F;
X₄ is a linker group of the formula:
—[CH₂]ⱼ₁-L₄-[CH₂]ⱼ₂—
wherein
j1 and j2 are selected from 0 or 1;
L₄ is selected from —O—, —S—, —SO—, —SO₂—, —N(Rₓ₄ₐ)— or —C(O)—, —C(O)O—, —OC(O)—, —C(O)N(Rₓ₄ₐ)— or —N(Rₓ₄ₐ)C(O)—; and wherein Rₓ₄ₐ and R_{x4b} are each independently selected from hydrogen or methyl;
Y₄ is selected from a phenyl, heteroaryl or heterocyclic ring which is optionally substituted by halo, nitro, cyano, R_{y4a}, —NR_{y4a}R_{y4b}, —OR_{y4a} or —C(O)R_{y4a};
wherein
R_{y4a} is hydrogen or methyl; and
R_{y4b} is hydrogen or methyl.

5. A compound according to any one of the preceding claims, or a pharmaceutically acceptable salt thereof, wherein R₁ is selected from:
(i) a group of the formula II: 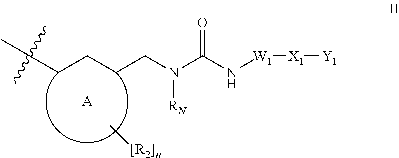
wherein:
Ring A is selected from phenyl or a 6-membered heteroaryl;
n is 0 or 1;
each R₂ group, when present, is selected from: halo, nitro, cyano, R₂ₐ, —NR₂ₐR_{2b}, —OR₂ₐ, —C(O)R₂ₐ;
R₂ₐ is hydrogen or methyl; and
R_{2b} is hydrogen or methyl;
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, methyl, halomethyl, cyano or amino;
W₁ is: 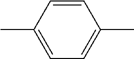
X₁ is a linker group of the formula:
—[CH₂]ₙ₁-L₁-[CH₂]ₙ₂—
wherein
n1 and n2 are selected from 0 or 1;
L₁ is selected from —O—, —S—, —SO₂— or —C(O)—;
Y₁ is selected from a phenyl, heteroaryl or heterocyclic ring which is optionally substituted by halo, nitro, cyano, R_{y1a} or —OR_{y1a};
R_{y1a} is hydrogen or methyl; and
R_{y1b} is hydrogen or methyl;
(ii) a group of the formula III: 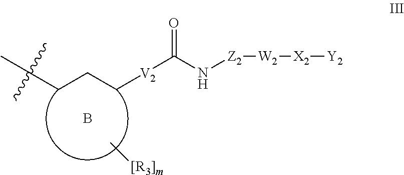
wherein:
Ring B is selected from phenyl or a 5- or 6-membered heteroaryl;
m is 0 or 1;
each R₃ group, when present, is selected from: halo, nitro, cyano, R₃ₐ, —NR₃ₐR_{3b}, —OR₃ₐ or —C(O)R₃ₐ;
R₃ₐ is hydrogen or methyl; and
R_{3b} is hydrogen or methyl;
V₂ is selected from —C(Rᵥ₂ₐR_{v2b})C(R_{v2c}R_{v2d})— or —C(Rᵥ₂ₐ)═C(R_{2c})—, wherein Rᵥ₂ₐ, R_{v2b}, R_{v2c} and R_{v2d} are each independently selected from hydrogen or methyl or Rᵥ₂ₐ and R_{v2c} are linked to form a cyclopropyl ring;
Z₂ is a 4C alkylene linker which is optionally substituted by one or more fluoro atoms;
W₂ is a carbocyclic or heterocyclic ring which is optionally substituted by halo, nitro, cyano, R_{w2a}, —NR_{w2a}R_{w2b}, —OR_{w2a} or —C(O)R_{w2a}; wherein
R_{w2a} is hydrogen or methyl; and
R_{w2b} and R_{w2c} are hydrogen or methyl;
X₂ is a linker group of the formula:
—[CH₂]ₘ₁-L₂-[CH₂]ₘ₂—
wherein
m1 and m2 are selected from 0 or 1;
L₂ is selected from —O—, —S—, —SO₂— or —C(O)—;
Y₂ is selected from a phenyl, heteroaryl or heterocyclic ring which is optionally substituted by halo, nitro, cyano, hydrogen, methyl or OR_{y2a};
R_{y2a} is hydrogen or methyl; and
R_{y2b} is hydrogen or methyl;
(iii) a group of the formula IV: 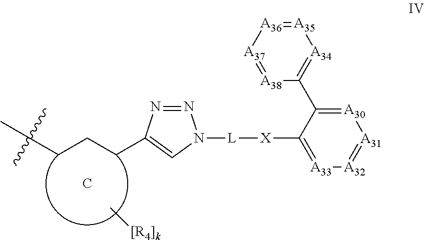
wherein:
Ring C is selected from phenyl or a 6-membered heteroaryl;
k is 0 or 1;
each R₄ group, when present, is selected from: halo, nitro, cyano, R₄ₐ, —NR₄ₐR_{4b}, —OR₄ₐ, —C(O)R₄ₐ;
R₄ₐ is hydrogen or methyl; and
R_{4b} is hydrogen or methyl;
L is a 6C alkylene linker which is optionally substituted by one or more fluoro atoms;
X is selected from —O—, —S—, —SO₂—, or —C(O)—;
A₃₀ and A₃₁ are selected from CH or C—F;
A₃₂ and A₃₃ are CH;
A₃₄ and A₃₅ are selected from CH or C—R₃₀;
A₃₆, A₃₇ and A₃₈ are CH;
and wherein R₃₀ is selected from halo, nitro, cyano, hydrogen, methyl, —NR₃₀ₐR_{30b}, —OR₃₀ₐ or —C(O)R₃₀ₐ; wherein
R₃₀ₐ is hydrogen or methyl; and
R_{30b} is hydrogen or methyl;
(iv) a group of the formula V or VI: 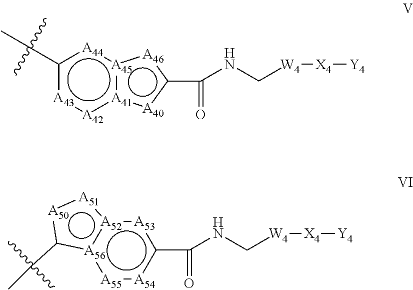
wherein:
A₄₀ is selected from NH or O;
A₄₂, A₄₃, A₄₄ and A₄₆ are each independently selected from CH or CR₂;
A₄₁ and A₄₅ are each C;
A₅₁ is selected from NH, NMe, CH or CR₂;
A₅₀, A₅₃, A₅₄ and A₅₅ are each independently selected from CH or CR₂;
A₅₂ and A₅₆ are each C;
wherein R₂ is as defined above;
W₄ is: 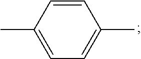
X₄ is a linker group of the formula:
-L₄-
wherein
L₄ is selected from —O—, —S—, —SO—, —SO₂—, —N(Rₓ₄ₐ)— or —C(O)—, —C(O)O—, —OC(O)—, —C(O)N(Rₓ₄ₐ)— or —N(Rₓ₄ₐ)C(O)—; and wherein Rₓ₄ₐ and R_{x4b} are each independently selected from hydrogen or methyl;
Y₄ is selected from a phenyl, heteroaryl or heterocyclic ring which is optionally substituted by halo, nitro, cyano or methyl.

6. A compound according to any one of the preceding claims, or a pharmaceutically acceptable salt thereof, wherein R₁ is selected from:
(i) a group of the formula II: 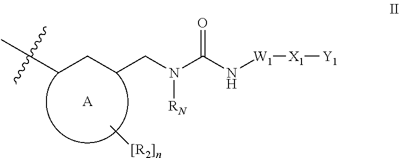
wherein:
Ring A is selected from phenyl or a 6-membered heteroaryl;
n is 0 or 1;
each R₂ group, when present, is selected from: halo or —OR₂ₐ;
wherein R₂ₐ is hydrogen or methyl; and
R_{N} is hydrogen
or a R₂ group and R_{N} are linked so as to form a 5- or 6-membered heterocyclic ring fused to Ring A, the fused 5- or 6-membered heterocyclic ring comprising one or two N atoms and being optionally substituted by halo, hydroxy, methyl, halomethyl, cyano or amino;
W₁ is: 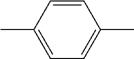
X₁ is a linker group of the formula:
—[CH₂]ₙ₁-L₁-[CH₂]ₙ₂—
wherein
n1 and n2 are selected from 0 or 1;
L₁ is selected from —O—, —S—, —SO₂— or —C(O)—;
Y₁ is selected from a phenyl, or heterocyclic ring which is optionally substituted by halo, nitro, cyano, hydrogen, methyl, or —OR_{y1a};
R_{y1a} is hydrogen or methyl;
(ii) a group of the formula III: 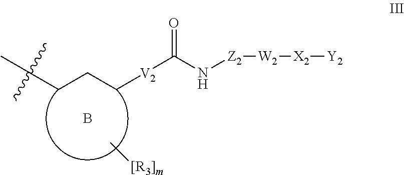
wherein:
Ring B is selected from phenyl or a 5- or 6-membered heteroaryl;
m is 0 or 1;
each R₃ group, when present, is selected from: halo, or —OR₃ₐ;
wherein R₃ₐ is hydrogen or methyl;
V₂ is selected from —C(Rᵥ₂ₐR_{v2b})C(R_{v2c}R_{v2d})— or —C(Rᵥ₂ₐ)═C(R_{v2c})—, wherein Rᵥ₂ₐ, R_{v2b}, R_{v2c} and R_{v2d} are each independently selected from hydrogen or methyl or Rᵥ₂ₐ and Rᵥ₂ are linked to form a cyclopropyl ring;
Z₂ is a 4C alkylene linker which is optionally substituted by one or more fluoro atoms;
W₂ is a heterocyclic ring which is optionally substituted by halo, or —OR_{w2a}; wherein
R_{w2a} is hydrogen or methyl;
X₂ is a linker group of the formula:
-L₂-
wherein
L₂ is selected from —O—, —S—, —SO₂— or —C(O)—;
Y₂ is a phenyl or heterocyclic ring which is optionally substituted by halo, nitro, cyano, hydrogen, methyl or OR_{y2a};
R_{y2a} is hydrogen or methyl;
(iii) a group of the formula IVa: 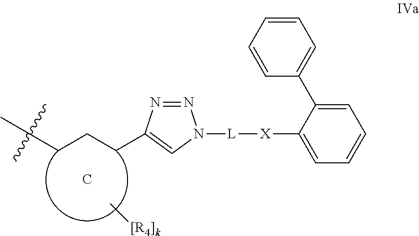
wherein:
Ring C is selected from phenyl or a 6-membered heteroaryl;
k is 0 or 1;
each R₄ group, when present, is selected from: halo, or —OR₄ₐ;
wherein R₄ₐ is hydrogen or methyl;
L is a 6C alkylene linker which is optionally substituted by one or more fluoro atoms;
X is selected from —O—, —S—, —SO₂—, or —C(O)—;
(iv) a group of the formula Va or VIa: 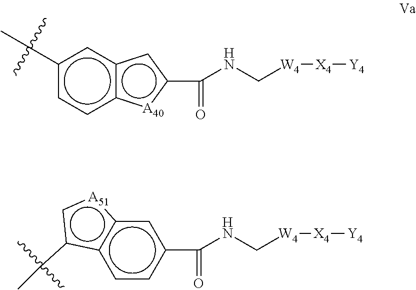
VIa
wherein:
A₄₀ is selected from NH or 0;
A₅₁ is selected from NH, NMe, CH or CR₂;
wherein R₂ is as defined above;
W₄ is: 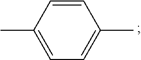
X₄ is a linker group of the formula:
-L₄-
wherein
L₄ is selected from —SO₂—, —C(O)N(Rₓ₄ₐ)— or —N(Rₓ₄ₐ)C(O)—; and wherein Rₓ₄ₐ is selected from hydrogen or methyl;
Y₄ is a phenyl or heterocyclic ring which is optionally substituted by halo, nitro, cyano or methyl.

7. A compound according to any one of the preceding claims, or a pharmaceutically acceptable salt thereof, wherein R₁ is selected from:
(i) a group of the formula II: 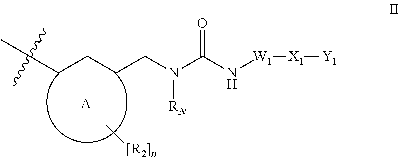
wherein:
Ring A is selected from phenyl or a 6-membered heteroaryl;
n is 0 or 1;
each R₂ group, when present, is selected from: halo or —OR₂ₐ;
wherein R₂ₐ is hydrogen or methyl; and
R_{N} is hydrogen
or a R₂ group and R_{N} are linked so as to form a 5-membered heterocyclic ring fused to Ring A, the fused 5-membered heterocyclic ring comprising one or two N atoms;
W₁ is: 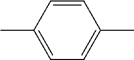
X₁ is a linker group of the formula:
—[CH₂]ₙ₁-L₁-[CH₂]ₙ₂—
wherein
n1 and n2 are selected from 0 or 1;
L₁ is selected from —SO₂—;
Y₁ is selected from a phenyl, or heterocyclic ring which is optionally substituted by halo, or —OR_{y1a};
wherein R_{y1a} is hydrogen or methyl;
(ii) a group of the formula III: 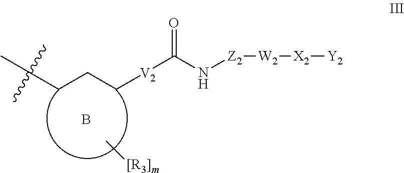
wherein:
Ring B is selected from phenyl or a 6-membered heteroaryl;
m is 0 or 1;
each R₃ group, when present, is selected from: halo, or —OR₃ₐ;
wherein R₃ₐ is hydrogen or methyl;
V₂ is selected from —C(Rᵥ₂ₐR_{v2b})C(R_{v2c}R_{v2d})— or —C(Rᵥ₂ₐ)═C(R_{2c})—, wherein Rᵥ₂ₐ, R_{v2b}, R_{v2c} and R_{v2d} are each hydrogen, or Rᵥ₂ₐ and R_{v2c} are linked to form a cyclopropyl ring;
Z₂ is a 4C alkylene linker;
W₂ is a heterocyclic ring which is optionally substituted by halo, or —OR_{w2a}; wherein R_{w2a} is hydrogen or methyl;
X₂ is a linker group of the formula:
-L₂-
wherein
L₂ is selected from —C(O)—;
Y₂ is a phenyl ring which is optionally substituted by halo, or OR_{y2a}; wherein
R_{y2a} is hydrogen or methyl;
(iii) a group of the formula IVa: 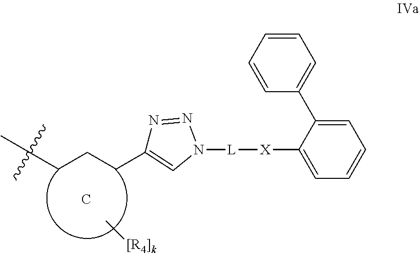
wherein:
Ring C is selected from phenyl or a 6-membered heteroaryl;
k is 0 or 1;
each R₄ group, when present, is selected from: halo, or —OR₄ₐ;
wherein R₄ₐ is hydrogen or methyl;
L is a 6C alkylene linker;
X is —O—;
(iv) a group of the formula Va or VIa: 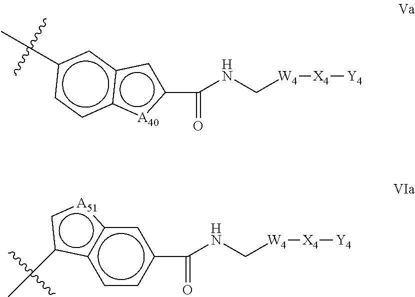
wherein:
A₄₀ is selected from NH or O;
A₅₁ is selected from NH, NMe, CH or CR₂;
wherein R₂ is as defined above
W₄ is: 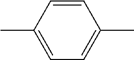
X₄ is a linker group of the formula:
-L₄-
wherein
L₄ is selected from —SO₂— or —C(O)N(Rₓ₄ₐ)—; and wherein Rₓ₄ₐ is independently selected from hydrogen or methyl;
Y₄ is a heterocyclic ring.

8. A compound according to any one of the preceding claims, or a pharmaceutically acceptable salt thereof, wherein R₁ is selected from:
(i) a group of the formula IIa or IIb: 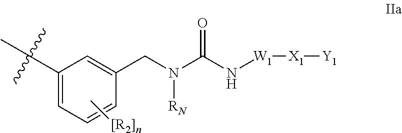 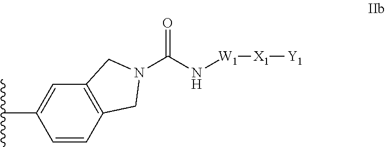
wherein:
Ring A is selected from phenyl or a 6-membered heteroaryl;
n is 0 or 1;
each R₂ group, when present, is selected from: halo or —OR₂ₐ;
wherein R₂ₐ is hydrogen or methyl; and
R_{N} is hydrogen
or a R₂ group and R_{N} are linked so as to form a 5-membered heterocyclic ring fused to Ring A, the fused 5-membered heterocyclic ring comprising one or two N atoms;
W₁ is: 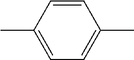
X₁ is a linker group of the formula:
—[CH₂]ₙ₁-L₁-[CH₂]ₙ₂—
wherein
n1 and n2 are selected from 0 or 1;
L₁ is selected from —SO₂—;
Y₁ is selected from a phenyl, or heterocyclic ring which is optionally substituted by halo or —OR_{y1a};
wherein R_{y1a} is hydrogen or methyl;
(ii) a group of the formula IIIa: 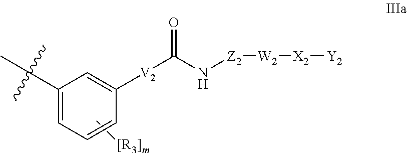
wherein:
m is 0 or 1;
each R₃ group, when present, is selected from: halo, or —OR₃ₐ;
wherein R₃ₐ is hydrogen or methyl;
V₂ is selected from —C(Rᵥ₂ₐR_{v2b})C(R_{v2c}R_{v2d})— or —C(Rᵥ₂ₐ)═C(R_{2c})—, wherein Rᵥ₂ₐ, R_{v2b}, R_{v2c} and R_{v2d} are each hydrogen, or Rᵥ₂ₐ and R_{v2c} are linked to form a cyclopropyl ring;
Z₂ is a 4C alkylene linker;
W₂ is a heterocyclic ring which is optionally substituted by halo, or —OR_{w2a};
wherein R_{w2a} is hydrogen or methyl;
X₂ is a linker group of the formula:
-L₂-
wherein
L₂ is selected from —C(O)—;
Y₂ is a phenyl ring which is optionally substituted by halo, or OR_{y2a}; wherein
R_{y2a} is hydrogen or methyl;
(iii) a group of the formula IVb: 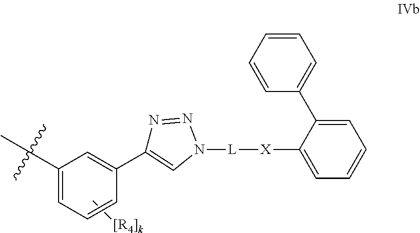
wherein:
k is 0 or 1;
each R₄ group, when present, is selected from: halo, or —OR₄ₐ;
wherein R₄ₐ is hydrogen or methyl;
L is a 6C alkylene linker;
X is —O—;
(iv) a group of the formula Vb, Vc, VIb or VIc: 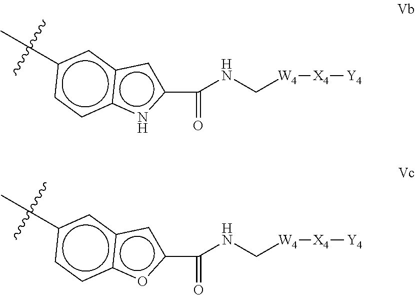 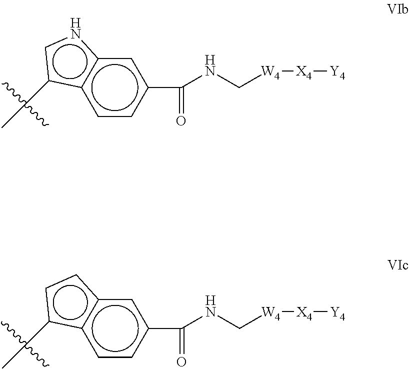
wherein:
W₄ is: 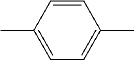
X₄ is a linker group of the formula:
-L₄-
wherein
L₄ is —SO₂—;
Y₄ is a 5 or 6 membered heterocyclic ring.

9. A compound according to any one of the preceding claims, or a pharmaceutically acceptable salt or solvate thereof, and, in particular, any of the following:
6-(2-fluoro-5-((3-(4-(piperidin-1-ylsulfonyl)phenyl)ureido)methyl)phenyl)-4-(trioxidaneylthio)naphthalene-2-sulfonic acid;
(E)-6-(5-(3-((4-(1-benzoylpiperidin-4-yl)butyl)amino)-3-oxoprop-1-en-1-yl)-2-fluorophenyl)-4-(trioxidaneylthio)naphthalene-2-sulfonic acid;
6-(2-fluoro-5-((3-(4-(phenylsulfonyl)phenyl)ureido)methyl)phenyl)-4-(trioxidaneylthio) naphthalene-2-sulfonic acid;
6-(3-(1-(6-([1,1′-biphenyl]-2-yloxy)hexyl)-1H-1,2,3-triazol-4-yl)phenyl)-4-(trioxidaneylthio) naphthalene-2-sulfonic acid;
6-(3-((3-(4-(piperidin-1-ylsulfonyl)phenyl)ureido)methyl)phenyl)-4-(trioxidaneylthio) naphthalene-2-sulfonic acid;
6-(4-fluoro-3-((3-(4-(piperidin-1-ylsulfonyl)phenyl)ureido)methyl)phenyl)-4-(trioxidaneylthio) naphthalene-2-sulfonic acid;
6-(2-methoxy-5-((3-(4-(piperidin-1-ylsulfonyl)phenyl)ureido)methyl)phenyl)-4-(trioxidaneylthio)naphthalene-2-sulfonic acid;
6-(5-((3-(4-((8-oxa-3-azabicyclo[3.2.1]octan-3-yl)sulfonyl)phenyl)ureido)methyl)-2-fluorophenyl)-4-(trioxidaneylthio)naphthalene-2-sulfonic acid;
6-(2-((4-(piperidin-1-ylsulfonyl)benzyl)carbamoyl)-1H-indol-5-yl)-4-(trioxidaneylthio) naphthalene-2-sulfonic acid;
(S)-6-(2-((4-(((Tetrahydrofuran-3-yl)methyl)carbamoyl)phenyl)carbamoyl)isoindolin-5-yl)-4-(trioxidaneylthio)naphthalene-2-sulfonic acid.

10. A pharmaceutical composition comprising a compound according to any one of claims 1 to 9, or a pharmaceutically acceptable salt thereof, and one or more pharmaceutically acceptable excipients.

11. A compound according to any one of claims 1 to 9, or a pharmaceutically acceptable salt thereof, or a pharmaceutical composition according to claim 10, for use in therapy.

12. A compound according to any one of claims 1 to 9, or a pharmaceutically acceptable salt thereof, or a pharmaceutical composition according to claim 10, for use in the treatment of:
(i) diabetes;
(ii) cardiovascular disease (e.g. pulmonary arterial hypertension, acute coronary syndrome (ACS), congestive heart failure (CHF) or left ventricular hypertrophy);
(iii) inflammatory bowel condition (e.g. inflammatory bowel disease (IBD), Crohn's Disease or ulcerative colitis)
(iv) cancer (e.g. breast cancer, prostate cancer or chronic lymphocytic leukaemia (CLL));
(v) liver disease (e.g. non-alcoholic fatty liver disease (NAFLD), in particular Non-Alcoholic SteatoHepatitis (NASH));
(vi) inflammatory skin conditions (e.g. psoriasis);
(vii) lung conditions (Acute Lung Injury (ALI), Ventilator Induced Lung Injury (VILI), Radiation-Induced Lung Injury (RILI), pulmonary fibrosis (e.g. idiopathic pulmonary fibrosis));
(viii) arthritis (e.g. osteoarthritis or rheumatoid arthritis);
(ix) kidney disease (e.g. chronic kidney disease); or
(x) sepsis.

13. A compound according to any one of claims 1 to 9, or a pharmaceutically acceptable salt thereof, or a pharmaceutical composition according to claim 10, for use in the treatment of:
(i) diabetes;
(ii) pulmonary arterial hypertension, acute coronary syndrome (ACS), congestive heart failure (CHF) or left ventricular hypertrophy;
(iii) inflammatory bowel disease (IBD), Crohn's Disease or ulcerative colitis;
(iv) breast cancer, prostate cancer or chronic lymphocytic leukaemia (CLL)
(v) non-alcoholic fatty liver disease (NAFLD), in particular Non-Alcoholic SteatoHepatitis (NASH), hepatic steatosis, fibrosis or cirrhosis);
(vi) psoriasis;
(vii) Acute Lung Injury (ALI), Ventilator Induced Lung Injury (VILI), Radiation-Induced Lung Injury (RILI), pulmonary fibrosis (e.g. idiopathic pulmonary fibrosis);
(viii) osteoarthritis or rheumatoid arthritis;
(ix) chronic kidney disease; or
(x) sepsis.

14. A compound according to any one of claims 1 to 9, or a pharmaceutically acceptable salt thereof, or a pharmaceutical composition according to claim 10, for use in the treatment of non-alcoholic fatty liver disease, for example hepatic steatosis, through inflammatory non-alcoholic steatohepatitis (NASH) to fibrosis or cirrhosis.
